SEQUENCE LISTING (220 sequences)

SEQ ID NO: 1 - protein, 27 aa, unidentified
VQVTPHHVLVDEYTGEWVDSQFINGKC

SEQ ID NO: 2 - protein, 14 aa, unidentified
DRGWGNGCGLFGKG

SEQ ID NO: 3 - protein, 17 aa, unidentified
GLIHLHQNIVDVQYLYG

SEQ ID NO: 4 - protein, 16 aa, unidentified
GLFGAIAGFIENGWEG

SEQ ID NO: 5 - protein, 22 aa, unidentified
GFTCTGVVTEAETYTNFVGYVT

SEQ ID NO: 6 - protein, 18 aa, unidentified
SLHNPYPDYRWLRTVKTT

SEQ ID NO: 7 - protein, 19 aa, unidentified
GDAAWDFGSVGGVFNSLGK

SEQ ID NO: 8 - protein, 19 aa, unidentified
GGTAWDFGSIGGVFTSVGK

SEQ ID NO: 9 - protein, 19 aa, unidentified
GDTAWDFGSLGGVFTSVGK

SEQ ID NO: 10 - protein, 19 aa, unidentified
GETAWDFGSVGGLLTSLGK

SEQ ID NO: 11 - protein, 19 aa, unidentified
LGDTAWDFGSIGGVFNSIG

SEQ ID NO: 12 - protein, 19 aa, unidentified
LGDTAWDFGSVGGIFTSLG

SEQ ID NO: 13 - protein, 19 aa, unidentified
LGDTAWDFGSVGGVFNSIG

SEQ ID NO: 14 - protein, 19 aa, unidentified
LGDTAWDFGSVGGIFNSVG

SEQ ID NO: 15 - protein, 19 aa, unidentified
LGDTAWDFGSVGGVFTSVG

SEQ ID NO: 16 - protein, 19 aa, unidentified
IGDDAWDFGSVGGILNSVG

SEQ ID NO: 17 - protein, 19 aa, unidentified
VGSAFWNSDQRFSAINLMD

SEQ ID NO: 18 - protein, 14 aa, unidentified
DRGWGNGCALFGKG

SEQ ID NO: 19 - protein, 19 aa, unidentified
TGEHSWDFGSTGGFFASVG

SEQ ID NO: 20 - protein, 19 aa, unidentified
LGDTAWDFGSVGGFFTSLG

SEQ ID NO: 21 - protein, 19 aa, unidentified
IGDDAWDFGSTGGIFNTIG

SEQ ID NO: 22 - protein, 20 aa, unidentified
SSAFWNSDEPFHFSNLISII

SEQ ID NO: 23 - protein, 20 aa, unidentified
GDDAWDFGSTGGIFNTIGKA

SEQ ID NO: 24 - protein, 20 aa, unidentified
SSAYWSSSEPFTSAGIMRIL

SEQ ID NO: 25 - protein, 20 aa, unidentified
GSSSWDFSSAGGFFGSIGKA

SEQ ID NO: 26 - protein, 20 aa, unidentified
GDAAWDFGSVGGFMTSIGRA

SEQ ID NO: 27 - protein, 14 aa, unidentified
DRGWGNHCGLFGKG

SEQ ID NO: 28 - protein, 20 aa, unidentified
GETAWDFGSAGGFFTSVGRG

SEQ ID NO: 29 - protein, 20 aa, unidentified
GEAAWDFGSAGGFFQSVGRG

SEQ ID NO: 30 - protein, 20 aa, unidentified
LGDTAWDFGSVGGVFNSLGK

SEQ ID NO: 31 - protein, 19 aa, unidentified
VGEHAWDFGSVGGMLSSVG

SEQ ID NO: 32 - protein, 20 aa, unidentified
VLGEHAWDFGSVGGVMTSIG

SEQ ID NO: 33 - protein, 19 aa, unidentified
IGEHAWDFGSAGGFFSSIG

SEQ ID NO: 34 - protein, 19 aa, unidentified
LGEHAWDFGSTGGFLSSIG

SEQ ID NO: 35 - protein, 19 aa, unidentified
VGEHAWDFGSVGGILSSVG

SEQ ID NO: 36 - protein, 14 aa, unidentified
DRGWGNHCGFFGKG

SEQ ID NO: 37 - protein, 19 aa, unidentified
IGEHAWDFGSAGGFLSSIG

SEQ ID NO: 38 - protein, 19 aa, unidentified
IGEHAWDFGSTGGFLTSVG

SEQ ID NO: 39 - protein, 19 aa, unidentified
IGEHAWDFGSTGGFLASVG

SEQ ID NO: 40 - protein, 19 aa, unidentified
LGDTAWDFGSIGGVFTSLG

SEQ ID NO: 41 - protein, 19 aa, unidentified
VGSSSWDFSSTSGFFSSVG

SEQ ID NO: 42 - protein, 19 aa, unidentified
VGRSSWDFSSAGGFFSSVG

SEQ ID NO: 43 - protein, 19 aa, unidentified
MGDTAWDFSSAGGFFTSVG

SEQ ID NO: 44 - protein, 14 aa, unidentified
NRGWGTGCFKWGIG

SEQ ID NO: 45 - protein, 14 aa, unidentified
NRGWGTGCFEWGLG

SEQ ID NO: 46 - protein, 23 aa, unidentified
HVAGRYSKHGMAGIGSVWEDLVR

SEQ ID NO: 47 - protein, 14 aa, unidentified
VDKYRRFGTAGVGG

SEQ ID NO: 48 - protein, 17 aa, unidentified
GLIHLHRNIVDVQYLYG

SEQ ID NO: 49 - protein, 17 aa, unidentified
GLIHLHQNIVDVQYMYG

SEQ ID NO: 50 - protein, 17 aa, unidentified
GLIHLHQNIVDTQYLYG

SEQ ID NO: 51 - protein, 24 aa, unidentified
NTTLLNGSAFQLICPYGWVGRVEC

SEQ ID NO: 52 - protein, 19 aa, unidentified
SYFQQYMLKGQYQYWFDLE

SEQ ID NO: 53 - protein, 24 aa, unidentified
NTTLLNGPAFQMVCPLGWTGTVSC

SEQ ID NO: 54 - protein, 19 aa, unidentified
SYFQQYMLKGEYQYWFDLE

SEQ ID NO: 55 - protein, 22 aa, unidentified
SLLNGPAFQMVCPQGWTGTIEC

SEQ ID NO: 56 - protein, 20 aa, unidentified
DRYFQQYMLKGKWQYWFDLD

SEQ ID NO: 57 - protein, 22 aa, unidentified
TLLNGSAFYLVCPIGWTGVIEC

SEQ ID NO: 58 - protein, 19 aa, unidentified
SYFQQYMLKGEYQYWFDLD

SEQ ID NO: 59 - protein, 22 aa, unidentified
TLLNGPAFQLVCPYGWTGTIEC

SEQ ID NO: 60 - protein, 23 aa, unidentified
DNYFQQYMLKGKYQYWFDLEATD

SEQ ID NO: 61 - protein, 22 aa, unidentified
TLLNGSAFQMVCPFGWTGQVEC

SEQ ID NO: 62 - protein, 23 aa, unidentified
DSYFQQYMLKGEYQYWFDLDAKD

SEQ ID NO: 63 - protein, 22 aa, unidentified
GVYPFMWGGAYCFCDTENTQVS

SEQ ID NO: 64 - protein, 23 aa, unidentified
APFGCEIYTNPIRAENCAVGSIP

SEQ ID NO: 65 - protein, 22 aa, unidentified
SDFGGIATVKYSASKSGKCAVH

SEQ ID NO: 66 - protein, 28 aa, unidentified
FSTANIHPEFRLQICTSYVTCKGDCHPP

SEQ ID NO: 67 - protein, 15 aa, unidentified
GVYPFMWGGAYCFCD

SEQ ID NO: 68 - protein, 19 aa, unidentified
GDCHPPKDHIVTHPQYHAQ

SEQ ID NO: 69 - protein, 12 aa, unidentified
AVSKTAWTWLTS

SEQ ID NO: 70 - protein, 17 aa, unidentified
VHCAAECHPPKDHIVNY

SEQ ID NO: 71 - protein, 20 aa, unidentified
PASHTTLGVQDISATAMSWV

SEQ ID NO: 72 - protein, 30 aa, unidentified
ACTFWAVNAYSSGGYAQLASYFNPGGSYYK

SEQ ID NO: 73 - protein, 27 aa, unidentified
QYHPTACEVEPAFGHSDAACWGFPTDT

SEQ ID NO: 74 - protein, 23 aa, unidentified
MSVFALASYVQHPHKTVRVKFHT

SEQ ID NO: 75 - protein, 14 aa, unidentified
ETRTVWQSVAGVSC

SEQ ID NO: 76 - protein, 22 aa, unidentified
NVTTEHPFCNMPHGQLEVQVPP

SEQ ID NO: 77 - protein, 20 aa, unidentified
DPGDLVEYIMNYTGNQQSRW

SEQ ID NO: 78 - protein, 23 aa, unidentified
GSPNCHGPDWASPVCQRHSPDCS

SEQ ID NO: 79 - protein, 15 aa, unidentified
RLVGATPERPRLRLV

SEQ ID NO: 80 - protein, 13 aa, unidentified
DADDPLLRTAPGP

SEQ ID NO: 81 - protein, 18 aa, unidentified
GEVWVTPVIGSQARKCGL

SEQ ID NO: 82 - protein, 22 aa, unidentified
TLLNGPAFQLVCPYGWTGTIEC

SEQ ID NO: 83 - protein, 19 aa, unidentified
LGDTAWDFGSVGGVLTSIG

SEQ ID NO: 84 - protein, 19 aa, unidentified
LGDTAWDFGSVGGIFNSIG

SEQ ID NO: 85 - protein, 14 aa, unidentified
NRGWNNGCGLFGKG

SEQ ID NO: 86 - protein, 14 aa, unidentified
HIRAGPYGHATVEM

SEQ ID NO: 87 - protein, 15 aa, unidentified
PEWIHAHTTSDPWHP

SEQ ID NO: 88 - protein, 18 aa, unidentified
PGPLGLKFKTVRPVALPR

SEQ ID NO: 89 - protein, 21 aa, unidentified
ALAPPRNVRVTGCYQCGTPAL

SEQ ID NO: 90 - protein, 20 aa, unidentified
EGLAPGGGNCHLTVNGEDVG

SEQ ID NO: 91 - protein, 20 aa, unidentified
EGLAPGGGNCHLTVNGEDVG

SEQ ID NO: 92 - protein, 23 aa, unidentified
RASARVIDPAAQSFTGVVYGTHT

SEQ ID NO: 93 - protein, 24 aa, unidentified
TAVSETRQTWAEWAAAHWWQLTLG

SEQ ID NO: 94 - protein, 20 aa, unidentified
NPPDCPGVGTGCTACGVYLD

SEQ ID NO: 95 - protein, 21 aa, unidentified
RKVCIQLGTEQTCKTIDSNDC

SEQ ID NO: 96 - protein, 33 aa, unidentified
DTLLFLGPLEEGGMIFKQWCTTTCQFGDPGDIM

SEQ ID NO: 97 - protein, 24 aa, unidentified
GSFRKKCSFATLPSCQYDGNTVSG

SEQ ID NO: 98 - protein, 15 aa, unidentified
ATKDSFQSFNITEPH

SEQ ID NO: 99 - protein, 17 aa, unidentified
GSGVGFNLVCSVSLTEC

SEQ ID NO: 100 - protein, 22 aa, unidentified
KACDSAMCYGSSTANLVRGQNT

SEQ ID NO: 101 - protein, 30 aa, unidentified
GKGGHSGSKFMCCHDKKCSATGLVAAAPHL

SEQ ID NO: 102 - protein, 19 aa, unidentified
DDGAPQCGVHCWFKKSGEW

SEQ ID NO: 103 - protein, 22 aa, unidentified
KHDELCTGPCPVNINHQTGWLT

SEQ ID NO: 104 - protein, 22 aa, unidentified
WGCEEFGCLAVSDGCVFGSCQD

SEQ ID NO: 105 - protein, 24 aa, unidentified
GNGVPRFDYLCHLASRKEVIVRKC

SEQ ID NO: 106 - protein, 14 aa, unidentified
SCAGCINCFQNIHC

SEQ ID NO: 107 - protein, 17 aa, unidentified
KTVSSELSCREGQSYWT

SEQ ID NO: 108 - protein, 13 aa, unidentified
GSFSPKCLSSRRC

SEQ ID NO: 109 - protein, 27 aa, unidentified
ENKCFEQCGGWGCGCFNVNPSCLFVHT

SEQ ID NO: 110 - protein, 21 aa, unidentified
WGSVSLSLDAEGISGSNSFSF

SEQ ID NO: 111 - protein, 13 aa, unidentified
RQGFLGEIRCNSE

SEQ ID NO: 112 - protein, 23 aa, unidentified
AHESCLRAPNLVSYKPMIDQLEC

SEQ ID NO: 113 - protein, 15 aa, unidentified
DPFVVFERGSLPQTR

SEQ ID NO: 114 - protein, 17 aa, unidentified
QAFSKGSVQADLTLMFD

SEQ ID NO: 115 - protein, 17 aa, unidentified
CDAAFLNLTGCYSCNAG

SEQ ID NO: 116 - protein, 19 aa, unidentified
CQILHFTVPEVEEEFMYSC

SEQ ID NO: 117 - protein, 12 aa, unidentified
STVVNPKSGSWN

SEQ ID NO: 118 - protein, 17 aa, unidentified
FFDWFSGLMSWFGGPLK

SEQ ID NO: 119 - protein, 16 aa, unidentified
GLFGAIAGFIEGGWTG

SEQ ID NO: 120 - protein, 16 aa, unidentified
GLFGAIAGFIEGGWQG

SEQ ID NO: 121 - protein, 16 aa, unidentified
GIFGAIAGFIENGWEG

SEQ ID NO: 122 - protein, 16 aa, unidentified
GLFGAIAGFIENGWQG

SEQ ID NO: 123 - protein, 16 aa, unidentified
GIFGAIAGFIEGGWTG

SEQ ID NO: 124 - protein, 16 aa, unidentified
GLFGAIAGFIEGGWSG

SEQ ID NO: 125 - protein, 16 aa, unidentified
GLFGAIAGFIEGGWPG

SEQ ID NO: 126 - protein, 16 aa, unidentified
GFFGAIAGFLEGGWEG

SEQ ID NO: 127 - protein, 16 aa, unidentified
GAIALGVATAAAVTAG

SEQ ID NO: 128 - protein, 17 aa, unidentified
FLGLILGLGAAVTAGVA

SEQ ID NO: 129 - protein, 15 aa, unidentified
TNEAVVSLTNGMSVL

SEQ ID NO: 130 - protein, 13 aa, unidentified
VIRFQQLNKRLLE

SEQ ID NO: 131 - protein, 10 aa, unidentified
REFSSNAGLT

SEQ ID NO: 132 - protein, 14 aa, unidentified
MLTDRELTSIVGGM

SEQ ID NO: 133 - protein, 16 aa, unidentified
YVIQLPLFGVMDTDCW

SEQ ID NO: 134 - protein, 21 aa, unidentified
CLARADNGWYCHNAGSLSYFP

SEQ ID NO: 135 - protein, 16 aa, unidentified
DTLKSLTVPVTSRECN

SEQ ID NO: 136 - protein, 21 aa, unidentified
YDCKISTSKTYVSTAVLTTMG

SEQ ID NO: 137 - protein, 13 aa, unidentified
VSCYGHNSCTVIN

SEQ ID NO: 138 - protein, 13 aa, unidentified
VSCYGHNSCTVIN

SEQ ID NO: 139 - protein, 34 aa, unidentified
PLSFPDDKFDVAIRDVEHSINQTRTFLKASDQLL

SEQ ID NO: 140 - protein, 25 aa, unidentified
KIMTSKTDISSSVITSIGAIVSCYG

SEQ ID NO: 141 - protein, 18 aa, unidentified
RSAIEDLLFDKVKLSDVG

SEQ ID NO: 142 - protein, 17 aa, unidentified
VPFYLNVQYRINGLGVT

SEQ ID NO: 143 - protein, 21 aa, unidentified
VLSQNQKLIANAFNNALHAIQ

SEQ ID NO: 144 - protein, 16 aa, unidentified
TNSALVKIQAVVNANA

SEQ ID NO: 145 - protein, 24 aa, unidentified
AEAQIDRLINGRLTALNAYVSQQL

SEQ ID NO: 146 - protein, 31 aa, unidentified
SAAQAMEKVNECVKSQSSRINFCGNGNHIIS

SEQ ID NO: 147 - protein, 14 aa, unidentified
APYGLYFIHFNYVP

SEQ ID NO: 148 - protein, 32 aa, unidentified
LQEAIKVLNHSYINLKDIGTYEYYVKWPWYVW

SEQ ID NO: 149 - protein, 23 aa, unidentified
NALINDQLIMKNHLRDIMGIPYC

SEQ ID NO: 150 - protein, 20 aa, unidentified
FTWTLSDSEGKDTPGGYCLT

SEQ ID NO: 151 - protein, 29 aa, unidentified
KCFGNTAIAKCNQKHDEEFCDMLRLFDFN

SEQ ID NO: 152 - protein, 24 aa, unidentified
MLQKEYMERQGKTPLGLVDLFVFS

SEQ ID NO: 153 - protein, 16 aa, unidentified
FNPLGFFPSHQLDPLF

SEQ ID NO: 154 - protein, 13 aa, unidentified
ADWDKNPNKDPWP

SEQ ID NO: 155 - protein, 20 aa, unidentified
MESITSGFLGPLLVLQAVFF

SEQ ID NO: 156 - protein, 25 aa, unidentified
LLTRILTIPQSLDSWWTSLNFLGGA

SEQ ID NO: 157 - protein, 12 aa, unidentified
CPPTCPGYRWMC

SEQ ID NO: 158 - protein, 18 aa, unidentified
LFILLLCLIFLLVLLDYQ

SEQ ID NO: 159 - protein, 15 aa, unidentified
ETRTVWQLSVAGVSC

SEQ ID NO: 160 - protein, 24 aa, unidentified
LDGYLCRKQKWEVTCTETWYFVTD

SEQ ID NO: 161 - protein, 14 aa, unidentified
KYQIIEVIPTENEC

SEQ ID NO: 162 - protein, 24 aa, unidentified
LKGEYIPPYYPPTNCVWNAIDTQE

SEQ ID NO: 163 - protein, 18 aa, unidentified
IEDPVTMTLMDSKFTKPC

SEQ ID NO: 164 - protein, 13 aa, unidentified
LHCQIKSWECIPV

SEQ ID NO: 165 - protein, 15 aa, unidentified
SHRNMMEALYLESPD

SEQ ID NO: 166 - protein, 20 aa, unidentified
LTFCGYNGILLDNGEWWSIY

SEQ ID NO: 167 - protein, 18 aa, unidentified
ELEHEKCLGTLEKLQNGE

SEQ ID NO: 168 - protein, 17 aa, unidentified
LDLSYLSPSNPGKHYAY

SEQ ID NO: 169 - protein, 15 aa, unidentified
IRAVCYYHTFSMNLD

SEQ ID NO: 170 - protein, 20 aa, unidentified
EWKTTCDYRWYGPQYITHSI

SEQ ID NO: 171 - protein, 15 aa, unidentified
LGFPPQSCGWASVTT

SEQ ID NO: 172 - protein, 23 aa, unidentified
KGSSIGKMFESTYRGAKRMAILG

SEQ ID NO: 173 - protein, 23 aa, unidentified
KGSSIGKMFEATARGARRMAILG

SEQ ID NO: 174 - protein, 23 aa, unidentified
KGSSIGQMFETTMRGAKRMAILG

SEQ ID NO: 175 - protein, 18 aa, unidentified
PALSTGLIHLHQNIVDVQ

SEQ ID NO: 176 - protein, 18 aa, unidentified
PALSTGLIHLHRNIVDVQ

SEQ ID NO: 177 - protein, 18 aa, unidentified
PALSTGLIHLHQNIVDTQ

SEQ ID NO: 178 - protein, 18 aa, unidentified
WTYNAELLVLLENERTLD

SEQ ID NO: 179 - protein, 18 aa, unidentified
NAELLVLLENERTLDYHD

SEQ ID NO: 180 - protein, 18 aa, unidentified
WTYNAELLVLMENERTLD

SEQ ID NO: 181 - protein, 18 aa, unidentified
NAELLVLMENERTLDYHD

SEQ ID NO: 182 - protein, 18 aa, unidentified
WSYNAELLVALENQHTID

SEQ ID NO: 183 - protein, 18 aa, unidentified
NAELLVALENQHTIDLTD

SEQ ID NO: 184 - protein, 18 aa, unidentified
NAELLVALENQHTIDVTD

SEQ ID NO: 185 - protein, 18 aa, unidentified
NAELLVLMENERTLDFHD

SEQ ID NO: 186 - protein, 18 aa, unidentified
NAELLVLLENERTLDMHD

SEQ ID NO: 187 - protein, 18 aa, unidentified
WSYNAELLVAMENQHTID

SEQ ID NO: 188 - protein, 18 aa, unidentified
NAELLVAMENQHTIDLAD

SEQ ID NO: 189 - protein, 18 aa, unidentified
WAYNAELLVLLENQKTLD

SEQ ID NO: 190 - protein, 18 aa, unidentified
NAELLVLLENQKTLDEHD

SEQ ID NO: 191 - protein, 18 aa, unidentified
WTYQAELLVAMENQHTID

SEQ ID NO: 192 - protein, 18 aa, unidentified
QAELLVAMENQHTIDMAD

SEQ ID NO: 193 - protein, 18 aa, unidentified
WSYNAQLLVLLENEKTLD

SEQ ID NO: 194 - protein, 18 aa, unidentified
NAQLLVLLENEKTLDLHD

SEQ ID NO: 195 - protein, 18 aa, unidentified
WSYNAKLLVLLENDKTLD

SEQ ID NO: 196 - protein, 18 aa, unidentified
NAKLLVLLENDKTLDMHD

SEQ ID NO: 197 - protein, 18 aa, unidentified
WSYNAKLLVLIENDRTLD

SEQ ID NO: 198 - protein, 18 aa, unidentified
NAKLLVLIENDRTLDLHD

SEQ ID NO: 199 - protein, 18 aa, unidentified
ISSQIELAVLLSNEGIIN

SEQ ID NO: 200 - protein, 18 aa, unidentified
QIELAVLLSNEGIINSED

SEQ ID NO: 201 - protein, 15 aa, unidentified
GLFGAAGFIENGWEG

SEQ ID NO: 202 - protein, 14 aa, unidentified
DRGWGNGCGDFGKG

SEQ ID NO: 203 - protein, 18 aa, unidentified
NAELLVLLENERTLDFHD

SEQ ID NO: 204 - protein, 19 aa, unidentified
GDTAWDFGSVGGVLNSLGK

SEQ ID NO: 205 - protein, 27 aa, unidentified
TLLNGPAFQLVCPYGWTGTIECDSYYQ

SEQ ID NO: 206 - protein, 18 aa, unidentified
QYIIKSGYQYWFDLTAKD

SEQ ID NO: 207 - protein, 14 aa, unidentified
LLNTPPPYQVSCGG

SEQ ID NO: 208 - protein, 18 aa, unidentified
WSYNAELLVAMENQHLAD

SEQ ID NO: 209 - protein, 36 aa, unidentified
GIIRTLPDGCHYISNKGVDRVQVGNTVYYLSKEVGK

SEQ ID NO: 210 - protein, 22 aa, unidentified
ESLVIISPSVADLDPYDRSLHS

SEQ ID NO: 211 - protein, 17 aa, unidentified
CKLKLCGVLGLRLMDGT

SEQ ID NO: 212 - protein, 16 aa, unidentified
ILGPDGNVLIPEMQSS

SEQ ID NO: 213 - protein, 17 aa, unidentified
QHMELLESSVIPLVHPL

SEQ ID NO: 214 - protein, 25 aa, unidentified
GLFGAIAGFIENGWEGCGGEKEKEK

SEQ ID NO: 215 - protein, 24 aa, unidentified
GLFGAAGFIENGWEGCGGEKEKEK

SEQ ID NO: 216 - protein, 22 aa, unidentified
GLFAGFIENGWEGCGGEKEKEK

SEQ ID NO: 217 - protein, 27 aa, unidentified
PALSTGLIHLHQNIVDVQCGGEKEKEK

SEQ ID NO: 218 - protein, 28 aa, unidentified
GDTAWDFGSIGGVFTSVGKCGGEKEKEK

SEQ ID NO: 219 - protein, 16 aa, unidentified
DRGWGNXCGXFGKGXX

SEQ ID NO: 220 - protein, 19 aa, unidentified
GDTAWDFGSVGGVLNSLGK

(19) United States
(12) Patent Application Publication
(10) Pub. No.: US 2024/0150409 A1
(43) Pub. Date: May 9, 2024

(54) Identification and Attenuation of the Immunosuppressive Domains in Fusion Proteins of Enveloped RNA Viruses

(71) Applicant: iSD Immunotech ApS, Copenhagen N (DK)

(72) Inventors: Mogens RYTTERGAARD DUCH, Risskov (DK); Shervin BAHRAMI, Aarhus C (DK)

(21) Appl. No.: 18/141,055

(22) Filed: Apr. 28, 2023

Related U.S. Application Data
(63) Continuation of application No. 17/078,877, filed on Oct. 23, 2020, now abandoned.
Application No. 17/078,877 is a division of application No. 15/179,005, filed on Jun. 10, 2016, now Pat. No. 10,961,279.
Application No. 15/179,005 is a division of application No. 14/350,151, filed on Apr. 7, 2014, now abandoned.
(60) Provisional application No. 61/544,441, filed on Oct. 7, 2011.

(30) Foreign Application Priority Data
Oct. 7, 2011 (DK) PA 2011 70564

(51) Int. Cl. C07K 14/005; A61K 39/12; C07K 1/00; C07K 4/02; C07K 7/08; C07K 14/08; C12N 7/00

(52) U.S. Cl. CPC C07K 14/005; A61K 39/12; C07K 1/00; C07K 4/02; C07K 7/08; C07K 14/08; C12N 7/00; C12N 2760/12121; C12N 2760/12122; C12N 2760/12221; C12N 2760/12222; C12N 2760/16121; C12N 2760/16122; C12N 2760/16134; C12N 2760/16221; C12N 2760/16222; C12N 2760/16234; C12N 2760/16321; C12N 2760/16322; C12N 2770/24121; C12N 2770/24122; C12N 2770/24134; C12N 2770/24221; C12N 2770/24222; C12N 2770/24234; C12N 2770/24321; C12N 2770/24322; C12N 2770/28021; C12N 2770/28034; C12N 2770/36121; C12N 2770/36122; C12N 2770/36221; C12N 2770/36222

(57) ABSTRACT

The present invention relates to enveloped RNA viruses. The invention in particular relates to the generation of superior antigens for mounting an immune response by first identifying then mutating the immunosuppressive domains in fusion proteins of enveloped RNA viruses resulting in decreased immunosuppressive properties of viral envelope proteins from the viruses.

### CROSS-REFERENCE TO RELATED APPLICATIONS

This application is a continuation of U.S. application Ser. No. 17/078,877 filed 23 Oct. 2020, which is a divisional application of U.S. application Ser. No. 15/179,005 filed 10 Jun. 2016, which is a divisional application of U.S. application Ser. No. 14/350,151 filed 7 Apr. 2014, now abandoned, which is the U.S. national phase of PCT/DK2012/050381 filed 5 Oct. 2012, which claims the benefit of U.S. Provisional Appl. No. 61/544,441 filed 7 Oct. 2011 and Danish Appl. No. PA 2011 70564 filed 7 Oct. 2011. Each of the aforementioned applications is hereby incorporated by reference in its entirety.

### FIELD OF THE INVENTION

The present invention relates to enveloped RNA viruses. In particular, the invention relates to the generation of superior antigens for mounting an immune response by first identifying then mutating the immunosuppressive domains in fusion proteins of enveloped RNA viruses resulting in decreased immunosuppressive properties of viral envelope proteins from said viruses.

### BACKGROUND OF THE INVENTION

### Classification of Viruses

### ICTV Classification

The International Committee on Taxonomy of Viruses (ICTV) developed the current classification system and wrote guidelines that put a greater weight on certain virus properties to maintain family uniformity. A unified taxonomy (a universal system for classifying viruses) has been established. The 7th ICTV Report formalized for the first time the concept of the virus species as the lowest taxon (group) in a branching hierarchy of viral taxa. However, at present only a small part of the total diversity of viruses has been studied, with analyses of samples from humans finding that about 20% of the virus sequences recovered have not been seen before, and samples from the environment, such as from seawater and ocean sediments, finding that the large majority of sequences are completely novel.

The general taxonomic structure is as follows:
Order (-virales)
Family (-viridae)
Subfamily (-virinae)
Genus (-virus)
Species (-virus)

In the current (2008) ICTV taxonomy, five orders have been established, the Caudovirales, Herpesvirales, Mononegavirales, Nidovirales, and Picornavirales. The committee does not formally distinguish between subspecies, strains, and isolates. In total there are 5 orders, 82 families, 11 subfamilies, 307 genera, 2,083 species and about 3,000 types yet unclassified.

### Baltimore Classification

The Baltimore Classification of viruses is based on the method of viral mRNA synthesis.

The ICTV classification system is used in conjunction with the Baltimore classification system in modern virus classification.

The Baltimore classification of viruses is based on the mechanism of mRNA production. Viruses must generate mRNAs from their genomes to produce proteins and replicate themselves, but different mechanisms are used to achieve this in each virus family. Viral genomes may be single-stranded (ss) or double-stranded (ds), RNA or DNA, and may or may not use reverse transcriptase (RT). Additionally, ssRNA viruses may be either sense (+) or antisense (−). This classification places viruses into seven groups:
I: dsDNA viruses (e.g. Adenoviruses, Herpesviruses, Poxviruses)
II: ssDNA viruses (+)sense DNA (e.g. Parvoviruses)
III: dsRNA viruses (e.g. Reoviruses)
IV: (+)ssRNA viruses (+)sense RNA (e.g. Picornaviruses, Togaviruses)
V: (−)ssRNA viruses (−)sense RNA (e.g. Orthomyxoviruses, Rhabdoviruses)
VI: ssRNA-RT viruses (+)sense RNA with DNA intermediate in life-cycle (e.g. Retroviruses)
VII: dsDNA-RT viruses (e.g. Hepadnaviruses)

As an example of viral classification, the chicken pox virus, varicella zoster (VZV), belongs to the order Herpesvirales, family Herpesviridae, subfamily Alphaherpesvirinae, and genus Varicellovirus. VZV is in Group I of the Baltimore Classification because it is a dsDNA virus that does not use reverse transcriptase.

Many viruses (e.g. influenza and many animal viruses) have viral envelopes covering their protein cores. The envelopes typically are derived from portions of the host cell membranes (phospholipids and proteins), but include some viral glycoproteins. Functionally, viral envelopes are used to enable viruses to enter host cells. Glycoproteins on the surface of the envelope serve to identify and bind to receptor sites on the host's membrane. Subsequently the viral envelope then fuses with that of the host's, allowing the viral capsid and viral genome to enter and infect the host.

Typically, in RNA viruses a single transmembrane glycoprotein, a fusion protein, undergoes a conformational transition triggered by receptor recognition or low pH, leading to the insertion of a fusion peptide into the plasma membrane or the membrane of an endocytic vesicle. For some RNA viruses, including members of the paramyxovirus family, separate envelope proteins mediate attachment and fusion.

Membrane fusion can occur either at the plasma membrane or at an intracellular location following internalization of virus by receptor-mediated endocytosis. Fusion is mediated by viral transmembrane proteins known as fusion proteins. Upon appropriate triggering, the fusion protein interacts with the target membrane through a hydrophobic fusion peptide and undergoes a conformational change that drives the membrane fusion reaction. There are a variety of fusion triggers, including various combinations of receptor binding, receptor/coreceptor binding, and exposure to the mildly acidic pH within the endocytic pathway. Fusion proteins from different viruses have different names in spite of the common functionality.

Based on important structural features, many virus membrane fusion proteins are currently annotated to either the “class I” membrane fusion proteins exemplified by the influenza hemagglutinin (HA) or HIV-1 gp41, or the “class II” proteins of the alphaviruses and flaviviruses. The alphaviruses and flaviviruses are members of the Togaviridae and Flaviviridae families, respectively. These small enveloped positive-sense RNA viruses are composed of a capsid protein that assembles with the RNA into the nucleocapsid, and a lipid bilayer containing the viral transmembrane (TM) proteins.

Class I fusion proteins are synthesized as single chain precursors, which then assemble into trimers. The polypeptides are then cleaved by host proteases, which is an essential step in rendering the proteins fusion competent. This proteolytic event occurs late in the biosynthetic process because the fusion proteins, once cleaved are metastable and readily activated. Once activated, the protein refolds into a highly stable conformation. The timing of this latter event is of crucial importance in the fusion process. Maintenance of the intact precursor polypeptide during folding and assembly of the oligomeric structure is essential if the free energy that is released during the refolding event is to be available to overcome the inherent barriers to membrane fusion. The new amino-terminal region that is created by the cleavage event contains a hydrophobic sequence, which is known as the fusion peptide. The authentic carboxy-terminal region of the precursor polypeptide contains the transmembrane anchor. In the carboxy-terminal polypeptide, there are sequences known as the heptad repeat that are predicted to have an alpha helical structure and to form a coiled coil structure. These sequences participate in the formation of highly stable structure that characterizes the post-fusion conformation of the fusion protein.

The class II fusion proteins are elongated finger-like molecules with three globular domains composed almost entirely of ß-sheets. Domain I is a ß-barrel that contains the N-terminus and two long insertions that connect adjacent ß-strands and together form the elongated domain II. The first of these insertions contains the highly conserved fusion peptide loop at its tip, connecting the c and d ß-strands of domain II (termed the cd loop) and containing 4 conserved disulfide bonds including several that are located at the base of the fusion loop. The second insertion contains the ij loop at its tip, adjacent to the fusion loop, and one conserved disulfide bond at its base. A hinge region is located between domains I and II. A short linker region connects domain Ito domain III, a ß-barrel with an immunoglobulin-like fold stabilized by three conserved disulfide bonds. In the full-length molecule, domain III is followed by a stem region that connects the protein to the virus TM anchor. Fitting of the structure of alphavirus E1 to cryo-electron microscopy reconstructions of the virus particle reveals that E1 is located almost parallel to the virus membrane, and that E1-E1 interactions form the an icosahedral lattice.

### Immunosuppressive Properties of Enveloped Viruses with Type I Fusion Proteins

Fusion proteins of a subset of enveloped Type I [1] viruses (retrovirus, lentivirus and filoviruses) have previously been shown to feature an immune suppressive activity. Inactivated retroviruses are able to inhibit proliferation of immune cells upon stimulation [2-4]. Expression of these proteins is enough to enable allogenic cells to grow to a tumor in immune competent mice. In one study, introduction of ENV expressing construct into MCA205 murine tumor cells, which do not proliferate upon s.c. injection into an allogeneic host, or into CL8.1 murine tumor cells (which overexpress class I antigens and are rejected in a syngeneic host) resulted in tumor growth in both cases [5]. Such immunosuppressive domains have been found in a variety of different viruses with type 1 fusion mechanism such as gamma-retroviruses like Mason pfeizer monkey virus (MPMV) and murine leukemia virus (MLV), lentiviruses such as HIV and in filoviruses such as Ebola and Marburg viruses [6-9].

This immune suppressive activity was in all cases located to a very well-defined structure within the class I fusion proteins, more precisely at the bend in the heptad repeat just N-terminale of the transmembrane structure in the fusion protein. The immunosuppressive effects range from significant inhibition of lymphocyte proliferation [7,8], cytokine skewing (up regulating IL-10; down regulating TNF-α, IL-12, IFN-γ) [10] and inhibition of monocytic burst [11] to cytotoxic T cell killing [12]. Importantly, peptides spanning ISD in these assays must either be linked as dimers or coupled to a carrier (i.e. >monomeric) to be active. Such peptides derived from immune-suppressive domains are able to reduce or abolish immune responses such as cytokine secretion or proliferation of T-cells upon stimulation. The protection mediated by the immunosuppressive properties of the fusion protein from the immune system of the host is not limited to the fusion protein but covers all the viral envelope proteins displayed at viral or cellular membranes in particular also the protein mediating attachment of the virus to the cell.

### Co-Location of the Immunosuppression Domain and the Fusion Domain

The immunosuppressive domain of retro-, lenti- and filoviruses overlap a structurally important part of the fusion subunits of the envelope proteins. Although the primary structure (sequence) of this part of the fusion proteins can vary greatly from virus to virus, the secondary structure, which is very well preserved among different virus families, is that of an alpha helix that bends in different ways during the fusion process This structure plays a crucial role during events that result in fusion of viral and cellular membranes. It is evident that the immunosuppressive domains of these (retroviral, lentiviral and filoviral) class I fusion proteins overlap with a very important protein structure needed for the fusion proteins mechanistic function.

The energy needed for mediating the fusion of viral and cellular membranes is stored in the fusion proteins, which are thus found in a meta-stable conformation on the viral surface. Once the energy is released to drive the fusion event, the protein will find its most energetically stable conformation. In this regard fusion proteins can be compared with loaded springs that are ready to be sprung. This high energy conformation makes the viral fusion proteins very susceptible to modifications; Small changes in the primary structure of the protein often result in the protein to be folded in its stable post fusion conformation. The two conformations present very different tertiary structures of the same protein.

It has been shown in the case of simple retroviruses that small structural changes in the envelope protein are sufficient to remove the immune suppressive effect without changing structure and hence the antigenic profile.

The mutated non-immune suppressive envelope proteins are much better antigens for vaccination. The proteins can induce a 30-fold enhancement of anti-env antibody titers when used for vaccination and are much better at launching an effective CTL response [6]. Furthermore, viruses that contain the non-immunosuppressive form of the friend murine leukemia virus envelope protein, although fully infectious in irradiated immunocompromised mice cannot establish an infection in immunocompetent animals. Interestingly in the latter group the non-immunosuppressive viruses induce both a higher cellular and humeral immune response, which fully protect the animals from subsequent challenge by wild type viruses [13].

Immunosuppressive domains in the fusion proteins (viral envelope proteins) from Retroviruses, lentiviruses and Filoviruses have been known since 1985 for retrovirus [7], since 1988 for lentivirus [8] and since 1992 for filoviruses [14]. These viruses, as mentioned above, all belong to enveloped RNA viruses with a type I fusion mechanism. The immunosuppressive domains of lentivirus, retroviruses and filoviruses show large structural similarity. Furthermore the immunosuppressive domain of these viruses are all located at the same position in the structure of the fusion protein, more precisely in the linker between the two heptad repeat structures just N-terminal of the transmembrane domain in the fusion protein. These heptad repeat regions constitute two alpha helices that play a critical role in the active mechanism of membrane fusion by these proteins. The immune suppressive domains can be located in relation to two well conserved cystein residues that are found in these structures. These cystein residues are between 4 and 6 amino acid residues from one another and in many cases are believed to form disulfide bridges that stabilize the fusion proteins. The immune suppressive domains in all three cases include at least some of the first 22 amino acids that are located N-terminal to the first cysteine residue. Recently the immunosuppressive domains in the fusion protein of these viruses have been successfully altered in such a way that the fusogenic properties of the fusion protein have been preserved. Such mutated fusion proteins with decreased immunosuppressive properties have been shown to be superior antigens for vaccination purposes [13].

### SUMMARY OF THE INVENTION

The inventors have been able to devise methods for the identification of new immunosuppressive domains or potentially immunosuppressive domains located in proteins displayed at the surface of enveloped RNA viruses. The inventors of the present invention have surprisingly found immunosuppressive domains or potentially immunosuppressive domains in fusion proteins in a large number of other enveloped RNA viruses in addition to lentivirus, retrovirus and filovirus, where such immunosuppressive domains had not been described previously. In addition, the inventors have been able to develop methods for mutating said immunosuppressive domains in order to reduce the immunosuppressive properties of viral surface proteins, which are useful for providing strategies for producing new vaccines with improved properties by making superior antigens, or for generation of neutralizing antibodies. Through such approaches, the inventors have been able to propose vaccination regimes against different types of viruses such as e.g. Hepatitis C, Dengue virus and Influenza where effective vaccination regimes have been in great demand for many years. This may allow the production of vaccines against virus for which no vaccines has been known e.g. hepatitis C and Dengue, as well as improved versions of known vaccines, e.g. for Influenza.

According to an aspect, the inventors propose the use of up to four parameters for the identification of immunosuppressive domain in enveloped RNA viruses with hitherto un-described immunosuppressive properties. Proposed parameters used as part of a strategy for identifying a peptide sequence or a peptide which likely acts as immunosuppressive domains may comprise one or more of the following parameters (preferably all parameters are used):
1): The peptide is preferably located in the fusion protein of enveloped RNA viruses;
2): The peptide is preferably capable of interacting with membranes;
3): Preferably a high degree of homology in the primary structure (sequence) of the peptide of said domain exists either within the Order, Family, Subfamily, Genus, or Species of viruses. This feature is due to the immunosuppressive domain being under a dual selection pressures, one as an immunosuppressive entity ensuring protection of the viral particle from the host immune system, another as a peptide interacting with membranes;
4): The position at the surface of the fusion protein at a given conformation is preferably a feature of immunosuppressive domains. This can be revealed either by position in a 3D structure or by antibody staining of cells expressing the fusion protein or on viral surfaces displaying the fusion protein.

Based upon these parameters the inventors have inter alia identified three new groups of enveloped RNA viruses with immunosuppressive domains in their fusion protein:
1: The inventors have identified immunosuppressive domains among enveloped RNA viruses with type II fusion mechanism. Hitherto, immunosuppressive domains have not been described for any enveloped RNA viruses with a type II fusion mechanism. Immunosuppressive domains have been identified by the inventors at two positions in two different groups of viruses:
   i. Co-localizing with the fusion peptide exemplified by the identification of an common immunosuppressive domain in the fusion peptide of Flavirius (Dengue virus, west Nile virus etc), and
   ii. In the hydrophobic alpha helix N-terminal of the transmembrane domain in the fusion protein exemplified by the finding of an immunosuppressive domain in said helixes of all flaviridae e.g. Hepatitis C virus, Dengue, west nile etc.
2: The inventors have identified immunosuppressive domains in the fusion protein among enveloped RNA viruses with type I fusion mechanism (excluding lentivirus, retrovirus and filovirus). This position co-localizes with the fusion peptide of said fusion protein as demonstrated by the identification of a common immunosuppressive domain in the fusion peptide of all Influenza A and B types.
3: The inventors have identified potential immunosuppressive domains located at various positions of type I enveloped RNA viruses (excluding lentivirus, retrovirus and filovirus) as well as in enveloped RNA viruses featuring a fusion protein with neither a type I nor a type II fusion structure.

After identification of the immunosuppressive domains these must be mutated in order to decrease or completely abrogate the immunosuppressive properties of the whole envelope protein (preferably both the attachment and fusion part of the envelope protein if these are separate proteins). Such viral envelope proteins with reduced immunosuppressive properties are ideal candidates for use as antigens in vaccine compositions or for the production of neutralizing antibodies.

According to an aspect, the invention concerns a method for identifying an immunosuppressive domain of an enveloped RNA virus containing a lipid membrane, said method comprising the following steps:
a. Identifying the fusion protein of said virus;
b. Identifying at least one well conserved domain preferably among the membrane associated domains of said fusion protein (where the immunosuppressive domain is preferably located at the surface of the protein in one or more of the different conformations of the fusion protein undergoing fusion);
c. Optionally multimerizing or dimerizing said peptide; and
d. Confirming the immunosuppressive activity of at least one optionally multimerized or dimerized peptide by testing said optionally dimerized or multimerized peptide for immunosuppresive activity.

Concerning step a., fusion proteins or putative fusion proteins are usually identified by searching scientific databases, e.g. such as searching NCBI taxonomy database (www.ncbi.nlm.nih.gov/Taxonomy/) and selecting proteins of the Family, Subfamily, Genus or Species to be investigated and subsequently searching these for fusion, or the specific fusion protein, such as the protein listed in Table 1 below.

Concerning step b., vira are divided according to the following classification: Order (-virales), Family (-viridae), Subfamily (-virinae), Genus (-virus), Species (-virus). In order to localize conserved regions in the fusion proteins one or a few candidates from all viruses within an order are aligned first using an alignment tool such as the cobalt alignment tool (www.ncbi.nlm.nih.gov/tools/cobalt/). If stretches of conserved amino acids, such as ranging from 6 to 30 amino acids long, can be identified these are considered as candidates for immunosuppressive regions and are subjected to further investigation. If no candidates are found in an order, the same procedure is applied to the family of viruses. If still no candidates are found by testing different viruses belonging to a family of viruses we move on to the subfamily of viruses. If we cannot localize regions of homology among the subfamily we then test viruses from a genus and if we still cannot localize regions of homology we ultimately align as many possible individual viral sequences from a single species of virus (up to 1400 individual viral sequences). In general regions of homology are identified by having at least 25%, more preferred at least 30%, preferably at least 40%, more preferred at least 50%, more preferred at least 60%, preferably at least 70%, and even more preferably at least 75% homology (i.e. sequence identity) within a given region.

Concerning step c., the dimerized peptide could be synthetic, the multimerized peptide could be displayed as dimerized or trimerized fusion proteins either displayed alone or on membranes such as a viral particle. Alternatively the multimerized peptides can be coupled to a carrier protein.

According to another aspect, the invention concerns a method for decreasing or completely abrogating the immunosuppressive properties of an immunosuppressive domain of a fusion protein of an enveloped RNA virus containing a lipid membrane, said method comprising the steps of:
e. Mutating an immunosuppressive domain to produce at least one, preferably a plurality of mutated peptides
f. Optionally dimerizing or multimerizing said at least one, preferably plurality of mutated peptides;
g. Selecting at least one of said, preferably a plurality of said mutated peptides by testing for reduced immunosuppressive properties, preferably as shown by at least 25% reduction as compared to a wildtype peptide mono-, di- or multimer corresponding to the multimerization status of said mutated peptides;
h. Mutating a fusion protein of an enveloped RNA virus containing a lipid membrane to contain said selected mutated peptide having reduced immunosuppressive properties;
i. Confirming expression by testing the whole viral envelope protein encompassing said mutated fusion protein for capability of being expressed by at least one of cellular or viral surfaces.

According to an aspect, the invention concerns a method, further comprising the step of:
j. Using said viral envelope protein encompassing said mutated fusion protein with reduced immunosuppressive properties as an antigen for generation of an enhanced immune response.

A number of strategies are proposed for knock-out (i.e. decreasing or completely abrogating) of the immunosuppressive domain, these strategies comprise, but are not limited to, mutating or modifying the immunosuppressive domain into having the sequence of a mutant. A knock-out may be achieved e.g. by mutation, deletion or insertion in an immunosuppressive domain. A mutation may be at least one exchange of an amino acid with another amino acid, at least one insertion, at least one deletion, or a combination of one or more of these.

Mutants decreasing or completely abrogating the immunosuppressive properties will be identified by performing a complete or partly scanning of said immunosuppressive peptide with either Isoleucine, Alanine Leucine, Asparagine, Lysine, Aspartic acid, Methionine, Cysteine, Phenylalanine, Glutamic acid, Threonine, Glutamine, Tryptophan, Glycine, Valine, Proline, Serine, Tyrosine, Arginine, Histidine, insertions, deletions or point mutations. Alternatively the literature will be searched for mutations in said regions where said mutation did not eliminate expression of the fusion protein on the surface of the cell or viral envelope. Dimerized versions of said mutants may be tested in a cell proliferation assay. The literature provides further details (as an example see Cross K J, Wharton S A, Skehel J J, Wiley D C, Steinhauer D A. Studies on influenza haemagglutinin fusion peptide mutants generated by reverse genetics. EMBO J. 2001 Aug. 15; 20(16):4432-42).

According to an aspect, the invention concerns a method for identifying an immunosuppressive domain in the fusion protein of an enveloped RNA virus having a lipid membrane, said method comprising:
a. Identifying at least one well conserved domain among the group consisting of the membrane associated domains of the fusion protein and the surface associated domains of the fusion protein;
b. Providing at least one peptide with the sequence of said identified at least one well conserved domain;
c. Optionally dimerizing or multimerizing said at least one peptide; and
d. Confirming the immunosuppressive activity of said at least one optionally dimerized or multimerized peptide by testing said at least one optionally dimerized or multimerized peptide for immunosuppressive activity.

According to another aspect, the invention concerns an immunosuppressive domain identified according to the invention.

According to another aspect, the invention concerns an immunosuppressive domain selected among the sequences of Table 1 and Seq. Id. 1-200.

According to an aspect, the invention concerns a method for decreasing or completely abrogating the immunosuppressive properties of an immunosuppressive domain of the fusion protein of an enveloped RNA virus having a lipid membrane, said method comprising the steps of:
e. Mutating an immunosuppressive domain to provide at least one mutated peptide;
f. Optionally dimerizing or multimerizing said at least one mutated peptide;
g. Selecting one of said optionally dimerized or multimerized mutated peptides showing reduced immunosuppressive properties;
h. Mutating the fusion protein of the enveloped RNA virus to contain said selected mutated peptide having reduced immunosuppressive properties;
i. Confirming expression by testing the viral envelope protein encompassing said mutated fusion protein for capability of being expressed by at least one of cellular or viral surfaces.

According to an aspect, the invention concerns a mutated peptide providing reduced immunosuppressive properties, said mutated peptide having a sequence according to Table 1 or any of Seq. Id. 201-203 or obtainable as said selected mutated peptide of the method according to the invention.

According to an aspect, the invention concerns a method for generating an enhanced immune response further comprising the step of:
j. Using said viral envelope protein encompassing said mutated fusion protein with reduced immunosuppressive properties as an antigen for generation of an enhanced immune response.

According to an aspect, the invention concerns a method for making an envelope protein having diminished immunosuppressive activity, comprising: Mutating or modifying an immunosuppressive domain, identifiable according to the invention, of an enveloped RNA virus with a lipid membrane surrounding the core, to include a peptide obtainable according to the invention.

According to an aspect, the invention concerns an envelope protein obtainable according to the invention.

According to an aspect, the invention concerns a mutated envelope protein obtainable according to the invention.

According to an aspect, the invention concerns a viral fusion protein from an enveloped RNA virus with reduced immunosuppressive properties, said fusion protein encompassing a mutated peptide, said mutated peptide displaying reduced immunosuppression, and said mutated peptide replacing an un-mutated wildtype peptide having a sequence of an ISU of Table 1 or is selected among Seq. Id. 1-200.

According to an aspect, the invention concerns an envelope protein comprising a mutated peptide according to the invention, said mutated fusion protein being displayed on the surface of cells wherein said mutated fusion protein is expressed.

According to an aspect, the invention concerns an enveloped RNA virus, different from a viruses selected among the group consisting of Retrovirus, Lentivirus and Filovirus, wherein an immunosuppressive domain has been modified or mutated to decrease or completely abrogate the immunosuppressive properties of an immunosuppressive domain of the fusion protein.

According to an aspect, the invention concerns a virus selected among the vira of Table 1, wherein an immunosuppressive domain has been modified or mutated to decrease or completely abrogate the immunosuppressive properties of an immunosuppressive domain of the fusion protein.

According to an aspect, the invention concerns an antigen obtainable by selecting a part of a mutated envelope protein according to the invention, said part comprising the mutated domain of said envelope protein.

According to an aspect, the invention concerns a nucleic acid sequence, preferably recombinant, encoding a mutated envelope protein, an envelope polypeptide or an antigen according to the invention.

According to an aspect, the invention concerns an isolated eukaryotic expression vector comprising a nucleic acid sequence according to the invention.

According to an aspect, the invention concerns a method for producing an antibody, said method comprising the steps of: Administering an entity selected among a mutated envelope, an envelope polypeptide, an antigen, a nucleic acid sequence or a vector according to the invention to a host, such as an animal; and obtaining the antibody from said host.

According to an aspect, the invention concerns an antibody obtainable according to the invention.

According to an aspect, the invention concerns neutralizing antibodies obtained or identified by the use of at least one envelope protein according to the invention.

According to an aspect, the invention concerns a method for manufacturing neutralizing antibodies comprising the use of at least one protein according to the invention.

According to an aspect, the invention concerns a method for manufacturing humanized neutralizing antibodies, comprising the use of at least one sequence selected among the sequences of Table 1 and sequences 201 to 203.

According to an aspect, the invention concerns a vaccine comprising a virus according to the invention.

According to an aspect, the invention concerns a vaccine composition comprising an envelope protein according to the invention.

According to an aspect, the invention concerns a vaccine composition comprising an entity selected among the group consisting of a mutated envelope protein, an envelope polypeptide, an antigen, a nucleic acid sequence, a vector and an antibody according to the invention, and in addition at least one excipient, carrier or diluent.

According to an aspect, the invention concerns a medical composition comprising antibodies raised using a virus according to the invention.

According to an aspect, the invention concerns a pharmaceutical composition comprising a mutated peptide, an envelope protein, a mutated envelope protein, an antigen, a nucleic acid sequence, a vector, an antibody or a vaccine composition according to the invention, and at least one pharmaceutically acceptable excipient, diluents or carrier.

According to an aspect, the invention concerns a use of a mutated peptide, an envelope protein, a mutated envelope protein, an antigen, a nucleic acid sequence, a vector or an antibody according to the invention, for a medical purpose, such as for the treatment, amelioration or prevention of a clinical condition, and/or such as for the manufacture of a medicament for the treatment, amelioration or prevention of a clinical condition.

According to an aspect, the invention concerns a method of treating or ameliorating the symptoms of an individual, or prophylactic treating an individual, comprising administering an amount of mutated peptide, an envelope protein, a mutated envelope protein, antigen, nucleic acid sequence, vector or vaccine composition according to the invention.

### BRIEF DESCRIPTION OF THE DRAWINGS

FIG. 1 shows the result of an experiment using Influenza derived peptides, and the effect of the dimeric pepides on proliferation of CTLL-2 cells. The peptides are coupled through an ss-bond involving the cysteine residues. The wt INF peptide inhibits synthesis of new DNA, whereas the non-IS #1 peptide has a much less and the non-IS #2 peptide no significant effect.

INF wt:
(SEQ ID NO: 214)
GLFGAIAGFIENGWEGCGGEKEKEK
INF non-IS #1:
(SEQ ID NO: 215)
GLFGAAGFIENGWEGCGGEKEKEK
INF non-IS #2:
(SEQ ID NO: 216)
GLFAGFIENGWEGCGGEKEKEK

FIG. 2 shows the result of two independent experiments on Flavi virus derived peptides.

FLV IS/1 and FLV IS/2 are two independent experiments using the dimerized peptide: In both cases, a significant inhibition of proliferation of CTLL-2 cells is evident, while the monomeric peptide has no effect.

FLV IS/1 and FLV IS/2: dimeric
(SEQ ID NO: 2)
DRGWGNGCGLFGKG
FLV IS mono/1: monomeric
(SEQ ID NO: 2)
DRGWGNGCGLFGKG

Control peptide: a dimerized non-immune suppressive control peptide.

The concentrations are given in μM.

FIG. 3 shows another experimental result. The dimeric peptide derived from Hepatits C surface protein inhibits proliferation of T-cells in a concentration dependent manner.

Hep C IS peptide has the sequence:

(SEQ ID NO: 217)
PALSTGLIHLHQNIVDVQCGGEKEKEK

FIG. 4 shows yet an experimental result. The effect of the dimeric pepides derived from Flavi viruses on proliferation of CTLL-2 cells. The peptides are coupled through an ss-bond using the cysteine residues. FLV FUS non-IS is representative of a non-immune suppressive mutant.

Den H3:
(SEQ ID NO: 218)
GDTAWDFGSIGGVFTSVGKCGGEKEKEK
FLV FUS non-IS:
(SEQ ID NO: 202)
DRGWGNGCGDFGKG

### DETAILED DESCRIPTION OF THE INVENTION

Table 1 provides a list of viruses and their immunosuppressive domain(s). Asterix denotes extremely conserved sequence in the immunosuppressive domain for a given class, group, family or species of viruses. New immunosuppressive domains identified and tested in CTLL-2 assay for a given class, group, family or species of viruses are listed. Both the columns with “Putative ISU as described in this application for identification of immunosuppressive domains” and “Peptides from domains from fusion proteins exhibiting immunosuppressive activity (ISU)” are candidates for domains which are immunosuppressive. Note that all of the entries of the latter column, were originally identified by the inventors as a member of the former column. Due to the redundancy, the entries of the latter column were not included in the former column.

1: The inventors have identified immunosuppressive domains in the fusion proteins among enveloped RNA viruses with a type II fusion mechanism. Insofar immunosuppressive domains have not been previously described for type II enveloped RNA viruses. The immunosuppressive domain has been identified at two positions in the fusion protein in two different groups of viruses A: Co-localizing with the fusion peptide exemplified by the identification of an common immunosuppressive domain in the fusion peptide of Flavirus (Dengue virus, westNile virus etc.) and B: in the hydrophobic alpha helix N-terminal of the transmembrane domain in the fusion protein exemplified by the finding of an immunosuppressive domain in said helixes of Flaviridae e.g. Hepatitis C virus, Dengue, WestNile virus etc, cf. Table 1.

2: The inventors have identified immunosuppressive domains in the fusion protein among enveloped RNA viruses with type I fusion mechanism (excluding lentivirus, retrovirus and filovirus). This new position co-localizes with the fusion peptide of said fusion protein as demonstrated by the identification of a common immunosuppressive domain in the fusion peptide of all Influenza A and B types, cf. Table 1.

3: The inventors have identified potential immunosuppressive domains located at various positions of type I enveloped RNA viruses (excluding lentivirus, retrovirus and filovirus) and enveloped RNA viruses with neither Type I nor type II fusion mechanism, cf. Table 1.

TABLE 1

| | | | | | Putative ISU as |
|---|---|---|---|---|---|
| | | | | | identified using |
| | | | | | the criteria |
| | | | | | described in this |
| | | | | | application for |
| | | | | | identification of |
| | | | Species | Species | immunosuppressive |
| Family | Genus | | (group) | (Strain) | domains |
| Flavi- | Flavi-virus | | Aroa virus | Bussuquara virus | SEQ ID NO: 85 |
| viridae | | | | guape virus | NRGWNNGCGLFGKG |
| | | | | Naranjal virus | ************** |
| | | | | | SEQ ID NO: 7 |
| | | | | | GDAAWDFGSVGGVFNSLGK |
| | | | | | **o****o*****oo*o** |
| | | | Dengue virus | Dengue 1 | SEQ ID NO: 8 |
| | | | | | GGTAWDFGSIGGVFTSVGK |
| | | | | | *o***************** |
| | | | | Dengue 2 | SEQ ID NO: 9 |
| | | | | | GDTAWDFGSLGGVFTSVGK |
| | | | | | ****************o** |
| | | | | | SEQ ID NO: 173 |
| | | | | | KGSSIGKMFEATARGARRMAILG |
| | | | | Dengue 3 | SEQ ID NO: 174 |
| | | | | | KGSSIGQMFETTMRGAKRMAILG |
| | | | | Dengue 4 | SEQ ID NO: 10 |
| | | | | | GETAWDFGSVGGLLTSLGK |
| | | | | | ************oo***** |
| | | | | | SEQ ID NO: 173 |
| | | | | | KGSSIGKMFEATARGARRMAILG |
| | | | Japanese | Japanese encephalitis virus | SEQ ID NO: 11 |
| | | | encephalitis | | LGDTAWDFGSIGGVFNSIG |
| | | | virus group | | ***o*************** |
| | | | | Koutango virus | SEQ ID NO: 12 |
| | | | | | LGDTAWDFGSVGGIFTSLG |
| | | | | Murray Valley encephalitis Virus | SEQ ID NO: 13 |
| | | | | | LGDTAWDFGSVGGVFNSIG |
| | | | | St. Louis encephalitis virus | SEQ ID NO: 11 |
| | | | | | LGDTAWDFGSIGGVFNSIG |
| | | | | | ******************* |
| | | | | Usutu virus | SEQ ID NO: 14 |
| | | | | | LGDTAWDFGSVGGIFNSVG |
| | | | | | ******************* |
| | | | | West Nile virus | SEQ ID NO: 15 |
| | | | | | LGDTAWDFGSVGGVFTSVG |
| | | | | | **********o******** |
| | | | Kokobera virus | Kokobera virus unclassified Kokobera | SEQ ID NO: 16 |
| | | | group | virus group | IGDDAWDFGSVGGILNSVG |
| | | | Modoc Virus group | Modoc Virus | SEQ ID NO: 17 |
| | | | | | VGSAFWNSDQRFSAINLMD |
| | | | | | SEQ ID NO: 18 |
| | | | | | DRGWGNGCALFGKG |
| | | | | Cowbone Ridge virus | |
| | | | | Jutiapa virus | |
| | | | | Sal Vieja virus | |
| | | | | San Perlita virus | |
| | | | mosquito-borne | Ilheus virus | SEQ ID NO: 84 |
| | | | viruses | | LGDTAWDFGSVGGIFNSIG |
| | | | | Sepik virus | SEQ ID NO: 19 |
| | | | | | TGEHSWDFGSTGGFFASVG |
| | | | Ntaya virus group | Bagaza virus | SEQ ID NO: 20 |
| | | | | | LGDTAWDFGSVGGFFTSLG |
| | | | | Tembusu virus | SEQ ID NO: 83 |
| | | | | | LGDTAWDFGSVGGVLTSIG |
| | | | | Yokose virus | SEQ ID NO: 21 |
| | | | | | IGDDAWDFGSTGGIFNTIG |
| | | | Rio Bravo virus | Apoi virus | SEQ ID NO: 22 |
| | | | group | | SSAFWNSDEPFHFSNLISII |
| | | | | Entebbe bat virus | SEQ ID NO: 23 |
| | | | | | GDDAWDFGSTGGIFNTIGKA |
| | | | | Rio Bravo virus | SEQ ID NO: 24 |
| | | | | | SSAYWSSSEPFTSAGIMRIL |
| | | | | Saboya virus | SEQ ID NO: 18 |
| | | | | | DRGWGNGCALFGKG |
| | | | | | SEQ ID NO: 25 |
| | | | | | GSSSWDFSSAGGFFGSIGKA |
| | | | Seaborne tick- | Meaban virus | SEQ ID NO: 26 |
| | | | borne virus group | | GDAAWDFGSVGGFMTSIGRA |
| | | | | | SEQ ID NO: 27 |
| | | | | | DRGWGNHCGLFGKG |
| | | | | Saumarez Reef virus | SEQ ID NO: 28 |
| | | | | | GETAWDFGSAGGFFTSVGRG |
| | | | | | SEQ ID NO: 27 |
| | | | | | DRGWGNHCGLFGKG |
| | | | | Tyuleniy virus | SEQ ID NO: 29 |
| | | | | | GEAAWDFGSAGGFFQSVGRG |
| | | | | | SEQ ID NO: 27 |
| | | | | | DRGWGNHCGLFGKG |
| | | | Spondweni virus | Zika virus | SEQ ID NO: 30 |
| | | | group | | LGDTAWDFGSVGGVFNSLGK |
| | | | | | *************oo**o** |
| | | | | Kyasanur forest disease virus | SEQ ID NO: 31 |
| | | | | | VGEHAWDFGSVGGMLSSVG |
| | | | | | *************o***** |
| | | | | | SEQ ID NO: 27 |
| | | | | | DRGWGNHCGLFGKG |
| | | | | Langat virus | SEQ ID NO: 32 |
| | | | | | VLGEHAWDFGSVGGVMTSIG |
| | | | | | SEQ ID NO: 27 |
| | | | | | DRGWGNHCGLFGKG |
| | | | | Louping ill virus | SEQ ID NO: 33 |
| | | | | | IGEHAWDFGSAGGFFSSIG |
| | | | | | **********o***oo*o* |
| | | | | | SEQ ID NO: 27 |
| | | | | | DRGWGNHCGLFGKG |
| | | | | Omsk hemorrhagic fever virus | SEQ ID NO: 34 |
| | | | | | LGEHAWDFGSTGGFLSSIG |
| | | | | | SEQ ID NO: 27 |
| | | | | | DRGWGNHCGLFGKG |
| | | | | Powassan virus | SEQ ID NO: 35 |
| | | | | | VGEHAWDFGSVGGILSSVG |
| | | | | | *************o***** |
| | | | | | SEQ ID NO: 36 |
| | | | | | DRGWGNHCGFFGKG |
| | | | | | ************* |
| | | | | Royal Farm virus | SEQ ID NO: 27 |
| | | | | | DRGWGNHCGLFGKG |
| | | | | Tick-borne encephalitis virus | SEQ ID NO: 37 |
| | | | | | IGEHAWDFGSAGGFLSSIG |
| | | | | | SEQ ID NO: 38 |
| | | | | | IGEHAWDFGSTGGFLTSVG |
| | | | | | SEQ ID NO: 39 |
| | | | | | IGEHAWDFGSTGGFLASVG |
| | | | | | SEQ ID NO: 27 |
| | | | | | DRGWGNHCGLFGKG |
| | | | Yaounde virus | | SEQ ID NO: 40 |
| | | | | | LGDTAWDFGSIGGVFTSLG |
| | | | Yellow fever | Banzi virus | SEQ ID NO: 41 |
| | | | virus group | | VGSSSWDFSSTSGFFSSVG |
| | | | | Bouboui virus | SEQ ID NO: 42 |
| | | | | | VGRSSWDFSSAGGFFSSVG |
| | | | | Edge Hill virus | |
| | | | | Uganda S virus | |
| | | | | Wesselsbron virus | |
| | | | | Yellow fever virus | SEQ ID NO: 43 |
| | | | | | MGDTAWDFSSAGGFFTSVG |
| | | | | | ***o*************** |
| | | | unclassified Flavivirus | Batu Cave virus | SEQ ID NO: 44 |
| | | | | Cacipacore virus | NRGWGTGCFKWGIG |
| | | | | Calbertado virus | SEQ ID NO: 45 |
| | | | | Cell fusing agent virus | NRGWGTGCFEWGLG |
| | | | | Chaoyang virus | |
| | | | | Chimeric Tick-borne encephalitis | |
| | | | | virus/Dengue virus 4 | |
| | | | | Culex theileri flavivirus | |
| | | | | Donggang virus | |
| | | | | Duck hemorrhagic ovaritis virus | |
| | | | | Flavivirus Aedes/MO-Ac/ITA/2009 | |
| | | | | Flavivirus Anopheles/PV-Am/ITA/2009 | |
| | | | | Flavivirus CbaAr4001 | |
| | | | | Flavivirus FSME | |
| | | | | Flavivirus Phlebotomine/76/Arrabida/2007 | |
| | | | | Gadgets Gully virus | |
| | | | | Greek goat encephalitis virus | |
| | | | | Jugra virus | |
| | | | | Kadam virus | |
| | | | | Kamiti River virus | |
| | | | | Kedougou virus | |
| | | | | Montana myotis leukoencephalitis virus | |
| | | | | Mosquito flavivirus | |
| | | | | Ngoye virus | |
| | | | | Nounane virus | |
| | | | | Phlebotomus flavivirus Alg_F19 | |
| | | | | Phlebotomus flavivirus Alg_F8 | |
| | | | | Quang Binh virus | |
| | | | | Russian Spring-Summer encephalitis virus | |
| | | | | Sokoluk virus | |
| | | | | Spanish sheep encephalitis virus | |
| | | | | T′Ho virus | |
| | | | | Tai forest virus B31 | |
| | | | | Tamana bat virus | |
| | | | | Tick-borne flavivirus | |
| | | | | Wang Thong virus | |
| | | | | Flavivirus sp. | |
| | | | | Aedes flavivirus | SEQ ID NO: 45 |
| | | | | | NRGWGTGCFEWGLG |
| | | | | | SEQ ID NO: 46 |
| | | | | | HVAGRYSKHGMAGIGSV |
| | | | | | WEDLVR |
| | | | | Culex flavivirus | SEQ ID NO: 44 |
| | | | | | NRGWGTGCFKWGIG |
| | | | | | SEQ ID NO: 47 |
| | | | | | VDKYRRFGTAGVGG |
| | Hepacivirus | | Hepatitis C virus | Hepatitis C virus genotype 1 a | |
| | | | | Hepatitis C virus genotype 1b | SEQ ID NO: 48 |
| | | | | | GLIHLHRNIVDVQYLYG |
| | | | | | SEQ ID NO: 176 |
| | | | | | PALSTGLIHLHRNIVDVQ |
| | | | | Hepatitis C virus genotype 2 | SEQ ID NO: 49 |
| | | | | | GLIHLHQNIVDVQYMYG |
| | | | | | SEQ ID NO: 175 |
| | | | | | PALSTGLIHLHQNIVDVQ |
| | | | | Hepatitis C virus genotype 3 | SEQ ID NO: 175 |
| | | | | | PALSTGLIHLHQNIVDVQ |
| | | | | Hepatitis C virus genotype 4 | SEQ ID NO: 175 |
| | | | | | PALSTGLIHLHQNIVDVQ |
| | | | | Hepatitis C virus genotype 5 | SEQ ID NO: 50 |
| | | | | | GLIHLHQNIVDTQYLYG |
| | | | | | SEQ ID NO: 177 |
| | | | | | PALSTGLIHLHQNIVDTQ |
| | | | | Hepatitis C virus genotype 6 | SEQ ID NO: 175 |
| | | | | | PALSTGLIHLHQNIVDVQ |
| | | | | All Hepatitis C virus | |
| | Pesti virus | | Border disease | Border disease virus - | SEQ ID NO: 51 |
| | | | virus | Border disease virus - X818 | NTTLLNGSAFQLICPYGWVGRVEC |
| | | | | Border disease virus 1 | SEQ ID NO: 52 |
| | | | | Border disease virus 2 | SYFQQYMLKGQYQYWFDLE |
| | | | | Border disease virus 3 | |
| | | | | Border disease virus isolates | |
| | | | Bovine viral | Bovine viral diarrhea virus 1-CP7 | SEQ ID NO: 53 |
| | | | diarrhea virus 1 | Bovine viral diarrhea virus 1-NADL | NTTLLNGPAFQMVCPLGWTGTVSC |
| | | | | Bovine viral diarrhea virus 1-Osloss | SEQ ID NO: 54 |
| | | | | Bovine viral diarrhea virus 1-SD1 | SYFQQYMLKGEYQYWFDLE |
| | | | | Bovine viral diarrhea virus isolates | |
| | | | | and strains | |
| | | | | Bovine viral diarrhea virus type 1a | |
| | | | | Bovine viral diarrhea virus type 1b | |
| | | | | Pestivirus isolate 97-360 | |
| | | | | Pestivirus isolate Hay 87/2210 | |
| | | | | Pestivirus strain mousedeer | |
| | | | | Pestivirus type 1 isolates | |
| | | | Bovine viral | Bovine viral diarrhea virus 2 | SEQ ID NO: 55 |
| | | | diarrhea virus 2 | Pestivirus sp. strain 178003 | SLLNGPAFQMVCPQGWTGTIEC |
| | | | (BVDV-2) | Pestivirus sp. strain 5250Giessen-3 | SEQ ID NO: 56 |
| | | | | Bovine viral diarrhea virus-2 | DRYFQQYMLKGKWQYWFDLD |
| | | | | isolate | |
| | | | | SCP | |
| | | | Classical swine | Classical swine fever virus | SEQ ID NO: 57 |
| | | | fever virus | Hog cholera virus strain Zoelen | TLLNGSAFYLVCPIGWTGVIEC |
| | | | | | SEQ ID NO: 58 |
| | | | | | SYFQQYMLKGEYQYWFDLD |
| | | | unclassified | Bovine viral diarrhea virus 3 | SEQ ID NO: 59 |
| | | | Pestivirus | | SEQ ID NO: 82 |
| | | | | | TLLNGPAFQLVCPYGWTGTIEC |
| | | | | | SEQ ID NO: 60 |
| | | | | | DNYFQQYMLKGKYQYWFDLEATD |
| | | | | Chamois pestivirus 1 | SEQ ID NO: 61 |
| | | | | | TLLNGSAFQMVCPFGWTGQVEC |
| | | | | | SEQ ID NO: 62 |
| | | | | | DSYFQQYMLKGEYQYWFDLDAKD |
| | | | | Porcine pestivirus isolate | SEQ ID NO: 205 |
| | | | | Bungowannah | TLLNGPAFQLVCPYGWTGTI |
| | | | | | ECDSYYQ |
| | | | | | SEQ ID NO: 206 |
| | | | | | QYIIKSGYQYWFDLTAKD |
| | Unnclassified | | | Barkedji virus | |
| | Flaviviridae | | | Canine hepacivirus AAK-2011 | |
| | | | | GB virus A | |
| | | | | Douroucouli hepatitis GB virus A | |
| | | | | GBV-A-like agents | |
| | | | | GB virus D | |
| | | | | GBV-C/HGV group | |
| | | | | GB virus C | |
| | | | | Hepatitis GB virus C-like virus | |
| | | | | Hepatitis GB virus B | |
| | | | | Lammi virus | |
| | | | | Marmoset hepatitis GB virus A | |
| | | | | Nakiwogo virus | |
| | | | | Turkey meningoencephalitis virus | |
| Toga- | Alpha-virus | | Aura virus | | SEQ ID NO: 63 |
| viridae | | | Barmah Forest | | GVYPFMWGGAYCFCDTENTQVS |
| | | | virus | | **********o****o**o*o* |
| | | | Middelburg virus | | SEQ ID NO: 64 |
| | | | Ndumu virus | | APFGCEIYTNPIRAENCAVGSIP |
| | | | Salmon pancreas | | *****o*ooo*o**oo*oo*oo* |
| | | | disease virus | | SEQ ID NO: 65 |
| | | | Getah virus | | SDFGGIATVKYSASKSGKCAVH |
| | | | Mayaro virus | | o***oooooo*ooooo*o*oo* |
| | | | Trocara virus | | SEQ ID NO: 66 |
| | | | EEEV complex | | FSTANIHPEFRLQICTSY |
| | | | | | VTCKGDCHPP |
| | | | WEEV complex | Fort Morgan virus | *oooooooo*oooo |
| | | | | Highlands J virus | *ooooo*ooo*o** |
| | | | | Sindbis virus | |
| | | | | Western equine encephalomyelitis | |
| | | | | virus | |
| | | | | Whataroa virus | |
| | | | | Cabassou virus | |
| | | | VEEV complex | Mucambo virus | |
| | | | | Pixuna virus | |
| | | | | Venezuelan equine encephalitis | |
| | | | | virus | SEQ ID NO: 67 |
| | | | | | GVYPFMWGGAYCFCD |
| | | | | | *************** |
| | | | | | SEQ ID NO: 68 |
| | | | | | GDCHPPKDHIVTHPQYHAQ |
| | | | | | ************o**o*o* |
| | | | | | SEQ ID NO: 69 |
| | | | | | AVSKTAWTWLTS |
| | | | | | *********oo* |
| | | | SFV complex | Bebaru virus | SEQ ID NO: 63 |
| | | | | O'nyong-nyong virus | GVYPFMWGGAYCFCDTENTQVS |
| | | | | Ross River virus | **********o****o**o*o* |
| | | | | Semliki forest virus | SEQ ID NO: 64 |
| | | | | Una virus | APFGCEIYTNPIRAENCAVGSIP |
| | | | | | *****o*ooo*o**oo*oo*oo* |
| | | | | | SEQ ID NO: 65 |
| | | | | | SDFGGIATVKYSASKSGKCAVH |
| | | | | | o***oooooo*ooooo*o*oo* |
| | | | | | SEQ ID NO: 66 |
| | | | | | FSTANIHPEFRLQICTSYV |
| | | | | | TCKGDCHPP |
| | | | | | *oooooooo*oooo*oooo |
| | | | | | o*ooo*o** |
| | | | | Chikungunya virus | SEQ ID NO: 67 |
| | | | | | GVYPFMWGGAYCFCD |
| | | | | | *************** |
| | | | | | SEQ ID NO: 70 |
| | | | | | VHCAAECHPPKDHIVNY |
| | | | | | oo*o*o**o******** |
| | | | | | SEQ ID NO: 71 |
| | | | | | PASHTTLGVQDISATAMSWV |
| | | | | | o****oo******o****** |
| | Rubivirus | | Rubella virus | Rubella virus (strain BRD1) | SEQ ID NO: 72 |
| | | | | Rubella virus (strain BRDII) | ACTFWAVNAYSSGGYAQLA |
| | | | | Rubella virus (strain Cendehill) | SYFNPGGSYYK |
| | | | | Rubella virus (strain M33) | ***o*o****o**oo**** |
| | | | | Rubella virus (strain RN-UK86) | o**o******o |
| | | | | Rubella virus (strain THERIEN) | SEQ ID NO: 73 |
| | | | | Rubella virus (strain TO-336 vaccine) | QYHPTACEVEPAFGHSDAACWGFPTDT |
| | | | | Rubella virus (strain TO-336) | ***o*o*o*o****o********o*** |
| | | | | Rubella virus (vaccine strain RA27/3) | SEQ ID NO: 74 |
| | | | | | MSVFALASYVQHPHKTVRVKFHT |
| | | | | | ***oo*****o**o**o****** |
| | | | | | SEQ ID NO: 159 |
| | | | | | ETRTVWQLSVAGVSC |
| | | | | | o*o*********oo* |
| | | | | | SEQ ID NO: 76 |
| | | | | | NVTTEHPFCNMPHGQLEVQVPP |
| | | | | | o*o*o**oo*o*o****o*oo* |
| | | | | | SEQ ID NO: 77 |
| | | | | | DPGDLVEYIMNYTGNQQSRW |
| | | | | | ****o******o*o****** |
| | | | | | SEQ ID NO: 78 |
| | | | | | GSPNCHGPDWASPVCQRHSPDCS |
| | | | | | ****o***o************** |
| | | | | | SEQ ID NO: 79 |
| | | | | | RLVGATPERPRLRLV |
| | | | | | o***o**o**o**** |
| | | | | | SEQ ID NO: 80 |
| | | | | | DADDPLLRTAPGP |
| | | | | | *oo********** |
| | | | | | SEQ ID NO: 81 |
| | | | | | GEVWVTPVIGSQARKCGL |
| | | | | | oo*o**o**o*****o** |
| | | | | | SEQ ID NO: 86 |
| | | | | | HIRAGPYGHATVEM |
| | | | | | oo***********o |
| | | | | | SEQ ID NO: 87 |
| | | | | | PEWIHAHTTSDPWHP |
| | | | | | o**oooo*o***o*o |
| | | | | | SEQ ID NO: 88 |
| | | | | | PGPLGLKFKTVRPVALPR |
| | | | | | ****o***o**o*oo*** |
| | | | | | SEQ ID NO: 89 |
| | | | | | ALAPPRNVRVTGCYQCGTPAL |
| | | | | | oooo**o*o*o**o******* |
| | | | | | SEQ ID NO: 90 and |
| | | | | | SEQ ID NO: 91 |
| | | | | | EGLAPGGGNCHLTVNGEDVG |
| | | | | | ***o*****o**oo*o*oo* |
| | | | | | SEQ ID NO: 207 |
| | | | | | LLNTPPPYQVSCGG |
| | | | | | ******o*o*o*** |
| | | | | | SEQ ID NO: 92 |
| | | | | | RASARVIDPAAQSFTGVVYGTHT |
| | | | | | **o***oo*o************* |
| | | | | | SEQ ID NO: 93 |
| | | | | | TAVSETRQTWAEWAAAHWWQLTLG |
| | | | | | o*******ooo*****o******* |
| Bunya- | Hanta-virus | | Amur virus | | SEQ ID NO: 94 |
| viridae | (continued | | Bayou virus | | NPPDCPGVGTGCTACGVYLD |
| | on next page) | | Black Creek Canal | | **o****o********o*** |
| | | | virus | | SEQ ID NO: 95 |
| | | | Cano Delgadito | | RKVCIQLGTEQTCKTIDSNDC |
| | | | virus | | *oo*o*o*o*oo**oo*v*** |
| | | | Calabazo virus | | SEQ ID NO: 96 |
| | | | Catacamas virus | | DTLLFLGPLEEGGMIFKQWC |
| | | | | | TTTCQFGDPGDIM |
| | | | Choclo virus | | SEQ ID NO: 97 |
| | | | Dobrava-Belgrade | | GSFRKKCSFATLPSCQYDGNTVSG |
| | | | virus | | *o***o*o***o*o*ooo**oo** |
| | | | El Moro Canyon | | SEQ ID NO: 98 |
| | | | virus | | ATKDSFQSFNITEPH |
| | | | Hantaan virus | | **o****o**oooo* |
| | | | Isla Vista virus | | SEQ ID NO: 99 |
| | | | Khabarovsk virus | | GSGVGFNLVCSVSLTEC |
| | | | Laguna Negra | | ******o*o*ooo**** |
| | | | virus | | SEQ ID NO: 100 |
| | | | Limestone Canyon | | KACDSAMCYGSSTANLVRGQNT |
| | | | virus | | ****o*o***ooooo*o**o** |
| | | | Monongahela virus | | SEQ ID NO: 101 |
| | | | Muleshoe virus | | GKGGHSGSKFMCCHDKKC |
| | | | | | SATGLVAAAPHL |
| | | | Muju virus | | ********o*o***ooo* |
| | | | | | ooo**o*oo*** |
| | | | New York virus | | SEQ ID NO: 102 |
| | | | Oran virus | | DDGAPQCGVHCWFKKSGEW |
| | | | Playa de Oro | | ***o*o*ooo***oo**** |
| | | | virus | | |
| | | | Prospect Hill | | |
| | | | virus | | |
| | | | Puumala virus | | |
| | | | Rio Mamore virus | | |
| | | | Rio Segundo virus | | |
| | | | Saaremaa virus | | |
| | | | Seoul virus | | |
| | | | Sin Nombre virus | | |
| | | | Soochong virus | | |
| | | | Thailand virus | | |
| | | | Thottapalayam | | |
| | | | virus | | |
| | | | Topografov virus | | |
| | | | Tula virus | | |
| | Ortho-bunya-virus | | Anopheles A virus | | SEQ ID NO: 103 |
| | | | Anopheles B virus | | KHDELCTGPCPVNINHQTGWLT |
| | | | Bakau virus | | *o*o***o**oooooooo*o*o |
| | | | Batama virus | | SEQ ID NO: 104 |
| | | | Bwamba virus | | WGCEEFGCLAVSDGCVFGSCQD |
| | | | Caraparu virus | | **o*oo**o*ooo**oo***** |
| | | | Kaeng Khoi virus | | SEQ ID NO: 105 |
| | | | Kairi virus | | GNGVPRFDYLCHLASRKEVIVRKC |
| | | | Madrid virus | | *o*ooo*ooo*oooo*ooooo*o* |
| | | | Main Drain virus | | SEQ ID NO: 106 |
| | | | Marituba virus | | SCAGCINCFQNIHC |
| | | | Nyando virus | | *o**ooooooooo* |
| | | | Oriboca virus | | |
| | | | Oropouche virus | | |
| | | | Sathuperi virus | | |
| | | | Shamonda virus | | |
| | | | Shuni virus | | |
| | | | Simbu virus | | |
| | | | Tacaiuma virus | | |
| | | | Tete virus | | |
| | | | Turlock virus | | |
| | | | unclassified | | |
| | | | Orthobunyavirus | | |
| | | | Akabane virus | Sabo virus | |
| | | | | Tinaroo virus | |
| | | | | Yaba-7 virus | |
| | | | Bunyamwera virus | Batai virus | |
| | | | | Birao virus | |
| | | | | Bozo virus | |
| | | | | Cache Valley virus | |
| | | | | Fort Sherman virus | |
| | | | | Germiston virus | |
| | | | | Guaroa virus | |
| | | | | Iaco virus | |
| | | | | Ilesha virus | |
| | | | | Lokern virus | |
| | | | | Maguari virus | |
| | | | | Mboke virus | |
| | | | | Ngari virus | |
| | | | | Northway virus | |
| | | | | Playas virus | |
| | | | | Potosi virus | |
| | | | | Shokwe virus | |
| | | | | Tensaw virus | |
| | | | | Tlacotalpan virus | |
| | | | | Xingu virus | |
| | | | California | California encephalitis serogroup virus | |
| | | | Encephalitis | LEIV | |
| | | | virus | California encephalitis virus - BFS-283 | |
| | | | | Chatanga virus | |
| | | | | Inkoo virus | |
| | | | | Jamestown Canyon virus | |
| | | | | Jamestown Canyon-like virus | |
| | | | | Jerry Slough virus | |
| | | | | Keystone virus | |
| | | | | La Crosse virus | |
| | | | | Lumbo virus | |
| | | | | Melao virus | |
| | | | | Morro Bay virus | |
| | | | | San Angelo virus | |
| | | | | Serra do Navio virus | |
| | | | | Snowshoe hare virus | |
| | | | | South River virus | |
| | | | | Tahyna virus | |
| | | | | Trivittatus virus | |
| | | | Caraparu virus | Apeu virus | |
| | | | | Bruconha virus | |
| | | | | Ossa virus | |
| | | | | Vinces virus | |
| | | | Manzanilla virus | Buttonwillow virus | |
| | | | | Ingwavuma virus | |
| | | | | Mermet virus | |
| | | | Marituba virus | Gumbo Limbo virus | |
| | | | | Murutucu virus | |
| | | | | Nepuyo virus | |
| | | | | Restan virus | |
| | | | Wyeomyia virus | Anhembi virus | |
| | | | | BeAr328208 virus | |
| | | | | Macaua virus | |
| | | | | Sororoca virus | |
| | | | | Taiassui virus | |
| | Phlebovirus | | Bujaru virus | | |
| | | | Candiruvirus | | |
| | | | Chilibre virus | | |
| | | | Frijoles virus | | |
| | | | Punta | | |
| | | | Tor_\|Salehabad | | |
| | | | virus | | |
| | | | Sandflyfever | | |
| | | | Naples virus | | |
| | | | Uukuniemi viruso | | |
| | | | virus | | |
| | | | Rift Valley | | SEQ ID NO: 107 |
| | | | fever virus | | KTVSSELSCREGQSYWT |
| | | | | | **oo**oo*o**o*o** |
| | | | | | SEQ ID NO: 108 |
| | | | | | GSFSPKCLSSRRC |
| | | | | | *******oooooo |
| | | | | | SEQ ID NO: 109 |
| | | | | | ENKCFEQCGGWGCGCFN |
| | | | | | VNPSCLFVHT |
| | | | | | **o*o**o*oo*oo*** |
| | | | | | ooo***o**o |
| | | | | | SEQ ID NO: 110 |
| | | | | | WGSVSLSLDAEGISGSNSFSF |
| | | | | | **ooo*o**o*o*o*o*oo** |
| | | | | | SEQ ID NO: 111 |
| | | | | | RQGFLGEIRCNSE |
| | | | | | *o*****o**oo* |
| | | | | | SEQ ID NO: 112 |
| | | | | | AHESCLRAPNLVSYKPMIDQLEC |
| | | | | | *oo**oo**oooo*o*oo*ooo* |
| | | | | | SEQ ID NO: 113 |
| | | | | | DPFVVFERGSLPQTR |
| | | | | | **ooo*oo*o***o* |
| | | | | | SEQ ID NO: 114 |
| | | | | | QAFSKGSVQADLTLMFD |
| | | | | | **ooo*ooo*oooooo* |
| | | | | | SEQ ID NO: 115 |
| | | | | | CDAAFLNLTGCYSCNAG |
| | | | | | *o*o*o*oo*****oo* |
| | | | | | SEQ ID NO: 116 |
| | | | | | CQILHFTVPEVEEEFMYSC |
| | | | | | *ooo*ooo*ooooooo*o* |
| | | | | | SEQ ID NO: 117 |
| | | | | | STVVNPKSGSWN |
| | | | | | *o*o**oooooo |
| | | | | | SEQ ID NO: 118 |
| | | | | | FFDWFSGLMSWFGGPLK |
| | | | | | *o***oo*o**oooooo |
| | | | unclassified | Anhanga virus | |
| | | | Phlebovirus (continued | Arumowot virus | |
| | | | on next page) | Chagres virus | |
| | | | | Corfou virus | |
| | | | | Gabek Forest virus | |
| | | | | Itaporanga virus | |
| | | | | Phlebovirus Adria/ALB1/2005 | |
| | | | | Phlebovirus Adria/ALB5/2005 | |
| | | | | Phlebovirus AH12 | |
| | | | | Phlebovirus AH12/China/2010 | |
| | | | | Phlebovirus AH15/China/2010 | |
| | | | | Phlebovirus B105-05 | |
| | | | | Phlebovirus B151-04 | |
| | | | | Phlebovirus B43-02 | |
| | | | | Phlebovirus B68-03 | |
| | | | | Phlebovirus B79-02 | |
| | | | | Phlebovirus Chios-A | |
| | | | | Phlebovirus Cyprus | |
| | | | | Phlebovirus HB29/China/2010 | |
| | | | | Phlebovirus HN13/China/2010 | |
| | | | | Phlebovirus HN6/China/2010 | |
| | | | | Phlebovirus Hu/Xinyang1/China/2010 | |
| | | | | Phlebovirus Hu/Xinyang2/China/2010 | |
| | | | | Phlebovirus IB13-04 | |
| | | | | Phlebovirus JS2007-01 | |
| | | | | Phlebovirus JS24 | |
| | | | | Phlebovirus JS26 | |
| | | | | Phlebovirus JS3/China/2010 | |
| | | | | Phlebovirus JS4/China/2010 | |
| | | | | Phlebovirus JS6 | |
| | | | | Phlebovirus JSD1 | |
| | | | | Phlebovirus LN2/China/2010 | |
| | | | | Phlebovirus LN3/China/2010 | |
| | | | | Phlebovirus sandflies/Gr29/Spain/2004 | |
| | | | | Phlebovirus sandflies/Gr36/Spain/2004 | |
| | | | | Phlebovirus sandflies/Gr44/Spain/2004 | |
| | | | | Phlebovirus sandflies/Gr49/Spain/2004 | |
| | | | | Phlebovirus sandflies/Gr52/Spain/2004 | |
| | | | | Phlebovirus sandflies/Gr65/Spain/2004 | |
| | | | | Phlebovirus sandflies/Gr98/Spain/2004 | |
| | | | | Phlebovirus SD24/China/2010 | |
| | | | | Phlebovirus SD4/China/2010 | |
| | | | | Phlebovirus tick/XCQ-2011 | |
| | | | | Phlebovirus XLL/China/2009 | |
| | | | | Rio Grande virus | |
| | | | | Salobo virus | |
| | | | | Sandfly fever sicilian virus | |
| | | | | Sandfly Sicilian Turkey virus | |
| | | | | Utique virus | |
| | | | | Phlebovirus sp. | |
| | | | | Phlebovirus sp. Be An 24262 | |
| | | | | Phlebovirus sp. Be An 356637 | |
| | | | | Phlebovirus sp. Be An 416992 | |
| | | | | Phlebovirus sp. Be An 578142 | |
| | | | | Phlebovirus sp. Be Ar 371637 | |
| | | | | Phlebovirus sp. Co Ar 170255 | |
| | | | | Phlebovirus sp. Co Ar 171616 | |
| | | | | Phlebovirus sp. GML 902878 | |
| | | | | Phlebovirus sp. Pa Ar 2381 | |
| | | | | Phlebovirus sp. PAN 479603 | |
| | | | | Phlebovirus sp. PAN 483391 | |
| | | | | Phlebovirus sp. VP-161A | |
| | | | | Phlebovirus sp. VP-334K | |
| | | | | Phlebovirus sp. VP-366G | |
| Ortho- | Influenzavirus A | | Influenza A virus | INFA H1 | SEQ ID NO: 119 |
| myxo- | | | | | GLFGAIAGFIEGGWTG |
| viridae | | | | | SEQ ID NO: 178 |
| | | | | | WTYNAELLVLLENERTLD |
| | | | | | SEQ ID NO: 179 |
| | | | | | NAELLVLLENERTLDYHD |
| | | | | INFA H2 | SEQ ID NO: 120 |
| | | | | | GLFGAIAGFIEGGWQG |
| | | | | | SEQ ID NO: 180 |
| | | | | | WTYNAELLVLMENERTLD |
| | | | | | SEQ ID NO: 181 |
| | | | | | NAELLVLMENERTLDYHD |
| | | | | INFA H3 | SEQ ID NO: 121 |
| | | | | | GIFGAIAGFIENGWEG |
| | | | | | SEQ ID NO: 182 |
| | | | | | WSYNAELLVALENQHTID |
| | | | | | SEQ ID NO: 183 |
| | | | | | NAELLVALENQHTIDLTD |
| | | | | INFA H4 | SEQ ID NO: 122 |
| | | | | | GLFGAIAGFIENGWQG |
| | | | | | SEQ ID NO: 182 |
| | | | | | WSYNAELLVALENQHTID |
| | | | | | SEQ ID NO: 184 |
| | | | | | NAELLVALENQHTIDVTD |
| | | | | INFA H5 | SEQ ID NO: 120 |
| | | | | | GLFGAIAGFIEGGWQG |
| | | | | | SEQ ID NO: 180 |
| | | | | | WTYNAELLVLMENERTLD |
| | | | | | SEQ ID NO: 185 |
| | | | | | NAELLVLMENERTLDFHD |
| | | | | INFA H6 | SEQ ID NO: 123 |
| | | | | | GIFGAIAGFIEGGWTG |
| | | | | | SEQ ID NO: 119 |
| | | | | | GLFGAIAGFIEGGWTG |
| | | | | | SEQ ID NO: 178 |
| | | | | | WTYNAELLVLLENERTLD |
| | | | | | SEQ ID NO: 186 |
| | | | | | NAELLVLLENERTLDMHD |
| | | | | INFA H7 | SEQ ID NO: 187 |
| | | | | | WSYNAELLVAMENQHTID |
| | | | | | SEQ ID NO: 208 |
| | | | | | WSYNAELLVAMENQHLAD |
| | | | | INFA H8 | SEQ ID NO: 124 |
| | | | | | GLFGAIAGFIEGGWSG |
| | | | | | SEQ ID NO: 189 |
| | | | | | WAYNAELLVLLENQKTLD |
| | | | | | SEQ ID NO: 190 |
| | | | | | NAELLVLLENQKTLDEHD |
| | | | | INFA H9 | SEQ ID NO: 125 |
| | | | | | GLFGAIAGFIEGGWPG |
| | | | | | SEQ ID NO: 124 |
| | | | | | GLFGAIAGFIEGGWSG |
| | | | | | SEQ ID NO: 189 |
| | | | | | WAYNAELLVLLENQKTLD |
| | | | | | SEQ ID NO: 190 |
| | | | | | NAELLVLLENQKTLDEHD |
| | | | | INFA H10 | SEQ ID NO: 191 |
| | | | | | WTYQAELLVAMENQHTID |
| | | | | | SEQ ID NO: 192 |
| | | | | | QAELLVAMENQHTIDMAD |
| | | | | INFA H11 | SEQ ID NO: 125 |
| | | | | | GLFGAIAGFIEGGWPG |
| | | | | | SEQ ID NO: 193 |
| | | | | | WSYNAQLLVLLENEKTLD |
| | | | | | SEQ ID NO: 194 |
| | | | | | NAQLLVLLENEKTLDLHD |
| | | | | INFA H12 | SEQ ID NO: 125 |
| | | | | | GLFGAIAGFIEGGWPG |
| | | | | | SEQ ID NO: 189 |
| | | | | | WAYNAELLVLLENQKTLD |
| | | | | | SEQ ID NO: 190 |
| | | | | | NAELLVLLENQKTLDEHD |
| | | | | INFA H13 | SEQ ID NO: 125 |
| | | | | | GLFGAIAGFIEGGWPG |
| | | | | | SEQ ID NO: 195 |
| | | | | | WSYNAKLLVLLENDKTLD |
| | | | | | SEQ ID NO: 196 |
| | | | | | NAKLLVLLENDKTLDMHD |
| | | | | INFA H14 | SEQ ID NO: 122 |
| | | | | | GLFGAIAGFIENGWQG |
| | | | | | SEQ ID NO: 182 |
| | | | | | WSYNAELLVALENQHTID |
| | | | | | SEQ ID NO: 184 |
| | | | | | NAELLVALENQHTIDVTD |
| | | | | INFA H15 | SEQ ID NO: 187 |
| | | | | | WSYNAELLVAMENQHTID |
| | | | | | SEQ ID NO: 188 |
| | | | | | NAELLVAMENQHTIDLAD |
| | | | | INFA H16 | SEQ ID NO: 125 |
| | | | | | GLFGAIAGFIEGGWPG |
| | | | | | SEQ ID NO: 197 |
| | | | | | WSYNAKLLVLIENDRTLD |
| | | | | | SEQ ID NO: 198 |
| | | | | | NAKLLVLIENDRTLDLHD |
| | Influenzavirus B | | Influenza B virus | All strains | SEQ ID NO: 126 |
| | | | | | GFFGAIAGFLEGGWEG |
| | | | | | SEQ ID NO: 199 |
| | | | | | ISSQIELAVLLSNEGIIN |
| | | | | | SEQ ID NO: 200 |
| | | | | | QIELAVLLSNEGIINSED |
| | Influenzavirus C | | Influenza C virus | | |
| Paramyxo- | Paramyxovirinae | | Avulavirus | Avian paramyxovirus 2 Yucaipa virus | SEQ ID NO: 127 |
| viridae | | | | Avian paramyxovirus 3 | GAIALGVATAAAVTAG |
| | | | | Avian paramyxovirus 3b | oooo*o*oo*o*oo** |
| | | | | Avian paramyxovirus 4 | |
| | | | | Avian paramyxovirus 5 | |
| | | | | Avian paramyxovirus 6 | |
| | | | | Avian paramyxovirus 7 | |
| | | | | Avian paramyxovirus 8 | |
| | | | | Avian paramyxovirus 9 | |
| | | | | Newcastle disease virus | |
| | | | | Pigeon paramyxovirus 1 | |
| | | | | unclassified Avulavirus | |
| | | | | Avian paramyxovirus 10_Avian | |
| | | | | paramyxovirus duck/Miyagi/885/05 | |
| | | | | Avian paramyxovirus penguin/Falkland | |
| | | | | Islands/324/2007 | |
| | | | | Goosramyxovirus HZ | |
| | | | | Goose paramyxovirus JS/1/97/Go | |
| | | | | Goose paramyxovirus SF02 | |
| | | | Henipavirus | Hendra virus Hendra virus | |
| | | | | horse/Australia/Hendra/1994 | |
| | | | | Nipah virus | |
| | | | | unclassified Henipavirus | |
| | | | | Bat paramyxovirus | |
| | | | | Eid.hel/GH45/2008 | |
| | | | Morbillivirus | Canine distemper virus | |
| | | | | Cetacean morbillivirus_Dolphin | |
| | | | | morbillivirus_Pilot whale morbillivirus | |
| | | | | Porpoise morbillivirus | |
| | | | | Measles virus | |
| | | | | Peste-des-petits-ruminants virus | |
| | | | | Phocine distemper virus | |
| | | | | Phocine distemper virus 1 | |
| | | | | Phocine distemper virus-2 | |
| | | | | Rinderpest virus | |
| | | | Respirovirus | Bovine parainfluenza virus 3 | |
| | | | | Porcine paramyxovirus strain Frost | |
| | | | | Porcine paramyxovirus strain Texas | |
| | | | | Human parainfluenza virus 1 | |
| | | | | Human parainfluenza virus 3 | |
| | | | | Simian Agent 10 | |
| | | | | Sendai virus | |
| | | | | unclassified Respirovirus | |
| | | | | Atlantic salmon respirovirus | |
| | | | | Guinea pig parainfluenza virus TS-9 | |
| | | | | Pacific salmon paramyxovirus | |
| | | | | Trask River 1983 Swine parainfluenza | |
| | | | | virus 3 | |
| | | | | Tursiops truncatus parainfluenza virus | |
| | | | | 1 | |
| | | | Rubulavirus | Human parainfluenza virus 2 | |
| | | | | Human parainfluenza virus 2 (strain | |
| | | | | Greer) | |
| | | | | Human parainfluenza virus 2 (strain | |
| | | | | Toshiba) | |
| | | | | Human parainfluenza virus 4 | |
| | | | | Human parainfluenza virus 4a | |
| | | | | Human parainfluenza virus 4b | |
| | | | | Mapuera virus | |
| | | | | Mumps virus | |
| | | | | Parainfluenza virus 5 | |
| | | | | Porcine rubulavirus | |
| | | | | Simian virus 41 | |
| | | | | unclassified Rubulavirus | |
| | | | | Porcine parainfluenza virus | |
| | | | | Tuhoko virus 1 | |
| | | | | Tuhoko virus 2 | |
| | | | | Tuhoko virus 3 | |
| | | | unclassified | Atlantic salmon paramyxovirus | |
| | | | Paramyxovirinae | Beilong virus | |
| | | | | Canine parainfluenza virus | |
| | | | | Chimeric human parainfluenza virus | |
| | | | | rPIV3-2 | |
| | | | | Fer-de-lance virus | |
| | | | | J-virus | |
| | | | | Menangle virus | |
| | | | | Mossman virus | |
| | | | | Murayama virus | |
| | | | | Ovine parainfluenza virus 3 | |
| | | | | Pacific salmon paramyxovirus | |
| | | | | Paramyxovirus GonoGER85 | |
| | | | | Recombinant PIV3/PIV1 virus | |
| | | | | Reptilian paramyxovirus | |
| | | | | Salem virus | |
| | | | | Salmo salar paramyxovirus | |
| | | | | Snake ATCC-VR-1408 paramyxovirus | |
| | | | | Snake ATCC-VR-1409 paramyxovirus | |
| | | | | Tioman virus | |
| | | | | Tupaia paramyxovirus | |
| | Pneumovirus | | Human respiratory | Human respiratory syncytial virus A | SEQ ID NO: 128 |
| | | | syncytial virus | Human respiratory syncytial virus | FLGLILGLGAAVTAGVA |
| | | | | (strain RSB1734) | ***oo**o*o*ooo*o* |
| | | | | Human respiratory syncytial virus | SEQ ID NO: 129 |
| | | | | (strain RSB5857) | TNEAVVSLTNGMSVL |
| | | | | Human respiratory syncytial virus | **o*****o**o*** |
| | | | | (strain RSB6190) | SEQ ID NO: 130 |
| | | | | Human respiratory syncytial virus | VIRFQQLNKRLLE |
| | | | | (strain RSB6256) | **o***o*o**** |
| | | | | Human respiratory syncytial virus | SEQ ID NO: 131 |
| | | | | (strain RSB642) | REFSSNAGLT |
| | | | | Human respiratory syncytial virus | ****o***o* |
| | | | | (strain RSB6614) | SEQ ID NO: 132 |
| | | | | Human respiratory syncytial virus A | MLTDRELTSIVGGM |
| | | | | strain Long LinkOut | ***oo**o*oooo* |
| | | | | Human respiratory syncytial virus A2 | SEQ ID NO: 133 |
| | | | | Human respiratory syncytial virus B | YVIQLPLFGVMDTDCW |
| | | | | Human respiratory syncytial | *oo***oo**o**o** |
| | | | | virus (subgroup B/strain 18537) | SEQ ID NO: 134 |
| | | | | Human respiratory syncytial virus | CLARADNGWYCHNAGSLSYFP |
| | | | | (subgroup B strain 8/60) | **ooo*o**o*o****o*o** |
| | | | | Human Respiratory syncytial virus 9320 | SEQ ID NO: 135 |
| | | | | Human respiratory syncytial virus B1 | DTLKSLTVPVTSRECN |
| | | | | Human respiratory syncytial virus S2 | **oo***o*ooooo** |
| | | | | Human respiratory syncytial virus | SEQ ID NO: 136 |
| | | | | strain RSS-2 | YDCKISTSKTYVSTAVLTTMG |
| | | | | unclassified Human respiratory | *o*o*o***ooo*oo*o*oo* |
| | | | | syncytial virus | SEQ ID NO: 137 |
| | | | | | VSCYGHNSCTVIN |
| | | | | | *****ooo**oo* |
| | | | | | SEQ ID NO: 209 |
| | | | | | GIIRTLPDGCHYISNKGVDR |
| | | | | | VQVGNTVYYLSKEVGK |
| | | | | | ***o*ooo**o*o**o* |
| | | | | | o*o*o****o**oo*oo** |
| | | | | | SEQ ID NO: 139 |
| | | | | | PLSFPDDKFDVAIRDVEHSIN |
| | | | | | QTRTFLKASDQLL |
| | | | | | **o**o*o*ooo*oo*oo |
| | | | | | o***ooo*ooo**o** |
| | | | | | SEQ ID NO: 140 |
| | | | | | KIMTSKTDISSSVITSIGAIVSCYG |
| | | | | | o*o***ooo*oo*o*oo*oo***** |
| | | | Bovine | All strains | SEQ ID NO: 128 |
| | | | respiratory | | FLGLILGLGAAVTAGVA |
| | | | syncytial virus | | ***oo**o*o*ooo*o* |
| | Metapneumovirus | | Avian metapneumo- | All strains | SEQ ID NO: 134 |
| | | | virus | | CLARADNGWYCHNAGSLSYFP |
| | | | | | **ooo*o**o*o****o*o** |
| | | | Human metapneumo- | All strains | SEQ ID NO: 133 |
| | | | virus | | YVIQLPLFGVMDTDCW |
| | | | | | *oo***oo**o**o** |
| Corona- | Coronavirinae | | Alphacorona-virus | Alphacoronavirus 1 | SEQ ID NO: 141 |
| viridae | | | | Coronavirus group 1b | RSAIEDLLFDKVKLSDVG |
| | | | | Human coronavirus 229E | **oo****oo**ooo*o* |
| | | | | Human coronavirus NL63 | SEQ ID NO: 142 |
| | | | | Miniopterus bat coronavirus 1 | VPFYLNVQYRINGLGVT |
| | | | | Miniopterus bat coronavirus HKU8 | o**ooooo**o**o*** |
| | | | | Porcine epidemic diarrhea virus | SEQ ID NO: 143 |
| | | | | Rhinolophus bat coronavirus HKU2 | VLSQNQKLIANAFNNALHAIQ |
| | | | | Scotophilus bat coronavirus 512 | **oo***o*ooo*oo*ooo** |
| | | | | unclassified Alphacoronavirus | SEQ ID NO: 144 |
| | | | Betacorona- | Betacoronavirus 1 | TNSALVKIQAVVNANA |
| | | | virus | Coronavirus group 2b | *oo**o*o*o***oo* |
| | | | | Coronavirus group 2c | SEQ ID NO: 145 |
| | | | | Human coronavirus HKU1 | AEAQIDRLINGRLTALNAYVSQQL |
| | | | | Murine coronavirus | *oo******o***oo*oo*oo*** |
| | | | | Pipistrellus bat coronavirus HKU5 | SEQ ID NO: 146 |
| | | | | Rousettus bat coronavirus HKU9 | SAAQAMEKVNECVKSQSSR |
| | | | | Severe acute respiratory syndrome- | INFCGNGNHIIS |
| | | | | related coronavirus recombinant SARSr- | o*oo*oo*oo***oo*oo* |
| | | | | CoV | oo***o*o*oo* |
| | | | | SARS coronavirus | SEQ ID NO: 147 |
| | | | | Tylonycteris bat coronavirus HKU4 | APYGLYFIHFNYVP |
| | | | | unclassified Betacoronavirus | **o*oo*o*oo*o* |
| | | | | | SEQ ID NO: 148 |
| | | | | | LQEAIKVLNHSYINLKDI |
| | | | | | GTYEYYVKWPWYVW |
| | | | | | oo*oo*o**o*ooo*ooo*oo* |
| | | | | | o*o*****o* |
| | | | Gammacorona-virus | Avian coronavirus | |
| | | | | Beluga Whale coronavirus SW1 | |
| | | | unclassified | Alpaca coronavirus CA08-1/2008 | |
| | | | coronaviruses | Bat coronavirus | |
| | | | | Bird droppings coronavirus | |
| | | | | Bovine respiratory coronavirus | |
| | | | | Chicken enteric coronavirus | |
| | | | | Coronavirus Anas | |
| | | | | Coronavirus oystercatcher/p17/2006/GBR | |
| | | | | Coronavirus red knot/p60/2006/GBR | |
| | | | | Ferret enteric coronavirus 1202 | |
| | | | | Ferret systemic coronavirus MSU-S | |
| | | | | Ferret systemic coronavirus WADL | |
| | | | | Guangxi coronaviridae | |
| | | | | Human coronavirus NO | |
| | | | | Human enteric coronavirus strain 4408 | |
| | | | | Kenya bat coronavirus | |
| | | | | Mink coronavirus strain WD1133 | |
| | | | | Parrot coronavirus AV71/99 | |
| | | | | Quail coronavirus Italy/Elvia/2005 | |
| | | | | Tai Forest coronavirus | |
| | | | | unidentified coronavirus | |
| | | | | unidentified human coronavirus | |
| Arena- | Arena-virus | | LCMV-Lassa virus | Ippy virus | SEQ ID NO: 149 |
| viridae | | | (Old World) | Lassa virus | NALINDQLIMKNHLRDIMGIPYC |
| | | | complex | Lujo virus | *o**o***o*o***o*o**o*** |
| | | | | Lymphocytic choriomeningitis virus | SEQ ID NO: 150 |
| | | | | Mobala virus | FTWTLSDSEGKDTPGGYCLT |
| | | | | Mopeia virus | oo*ooo*oo*ooo***o**o |
| | | | | | SEQ ID NO: 151 |
| | | | | | KCFGNTAIAKCNQKHDEEF |
| | | | | | CDMLRLFDFN |
| | | | | | ***o*ooo****oo* |
| | | | | | oo****ooo*ooo* |
| | | | | | SEQ ID NO: 152 |
| | | | | | MLQKEYMERQGKTPLGLVDLFVFS |
| | | | | | *ooo*oo**oo**oo*o*oooo*o |
| | | | Tacaribe virus | Amapari virus | SEQ ID NO: 150 |
| | | | (New World) | Chapare virus | FTWTLSDSEGKDTPGGYCLT |
| | | | complex | Flexal virus | oo*ooo*oo*ooo***o**o |
| | | | | Guanarito virus | SEQ ID NO: 151 |
| | | | | Junin virus | KCFGNTAIAKCNQKHD |
| | | | | Latino virus | EEFCDMLRLFDFN |
| | | | | Machupo virus | ***o*ooo****oo* |
| | | | | Oliveros virus | oo****ooo*ooo* |
| | | | | Parana virus | SEQ ID NO: 152 |
| | | | | Pichinde virus | MLQKEYMERQGKTPLGLVDLFVFS |
| | | | | Pirital virus | *ooo*oo**oo**oo*o*oooo*o |
| | | | | Sabia virus | |
| | | | | Tacaribe virus | |
| | | | | Tamiami virus | |
| | | | | Whitewater Arroyo virus | |
| Hepadna- | Genus | | Hepatitis B virus | HBV genotype A | SEQ ID NO: 153 |
| viridae | Orthohepadnavirus | | | HBV genotype B | FNPLGFFPSHQLDPLF |
| | | | | HBV genotype C | o***o*o*o*o*o*o* |
| | | | | HBV genotype D | SEQ ID NO: 154 |
| | | | | HBV genotype E | ADWDKNPNKDPWP |
| | | | | HBV genotype F | o*o*o*oo*oooo |
| | | | | HBV genotype G | SEQ ID NO: 155 |
| | | | | HBV genotype H | MESITSGFLGPLLVLQAVFF |
| | | | | Hepatitis B virus alpha1 | oooooooo*ooooo**oooo |
| | | | | Hepatitis B virus LSH/chimpanzee | SEQ ID NO: 156 |
| | | | | Hepatitis B virus strain cpz | LLTRILTIPQSLDSWWTSLNFLGGA |
| | | | | Hepatitis B virus subtype adr | oooooo*oooo*oooo***o*o*oo |
| | | | | Hepatitis B virus subtype adw | SEQ ID NO: 157 |
| | | | | Hepatitis B virus subtype adyw | CPPTCPGYRWMC |
| | | | | Hepatitis B virus subtype ayw | oo*o*****o*o |
| | | | | | SEQ ID NO: 158 |
| | | | | | LFILLLCLIFLLVLLDYQ |
| | | | | | *oo*ooo*oo*oo*oooo |
| Rhabdo- | Dimarhabdovirus | | Ephemerovirus | Bovine ephemeral fever virus | SEQ ID NO: 160 |
| viridae | | | | | LDGYLCRKQKWEVTCTETWYFVTD |
| | | | | | *o*oo****o*ooo*o*****o*o |
| | | | | | SEQ ID NO: 161 |
| | | | | | KYQIIEVIPTENEC |
| | | | | | o***o**o*oooo* |
| | | | | | SEQ ID NO: 162 |
| | | | | | LKGEYIPPYYPPTNCVWNAIDTQE |
| | | | | | oo*oo*******oo*o**oooo** |
| | | | | | SEQ ID NO: 163 |
| | | | | | IEDPVTMTLMDSKFTKPC |
| | | | | | ooo*oooooo**o*oo** |
| | | | | | SEQ ID NO: 164 |
| | | | | | LHCQIKSWECIPV |
| | | | | | o**oo*o****o* |
| | | | | | SEQ ID NO: 165 |
| | | | | | SHRNMMEALYLESPD |
| | | | | | *oo*oo*o*oo*o** |
| | | | | | SEQ ID NO: 166 |
| | | | | | LTFCGYNGILLDNGEWWSIY |
| | | | | | o****oo**oooo****** |
| | | | | | SEQ ID NO: 167 |
| | | | | | ELEHEKCLGTLEKLQNGE |
| | | | | | *****o**o*oo*oo*o* |
| | | | | | SEQ ID NO: 168 |
| | | | | | LDLSYLSPSNPGKHYAY |
| | | | | | **o***o*oo**oo*** |
| | | | | | SEQ ID NO: 169 |
| | | | | | IRAVCYYHTFSMNLD |
| | | | | | o**o*o*oo*oooo* |
| | | | Vesiculovirus | Carajas virus | SEQ ID NO: 170 |
| | | | | Chandipura virus | EWKTTCDYRWYGPQYITHSI |
| | | | | Cocal virus | o*o****o*****o*o*o* |
| | | | | Isfahan virus | SEQ ID NO: 171 |
| | | | | Maraba virus | LGFPPQSCGWASVTT |
| | | | | Piry virus | o****oo**oooooo |
| | | | | recombinant Vesiculovirus | SEQ ID NO: 1 |
| | | | | Spring viraemia of carp virus | VQVTPHHVLVDEYTGEW |
| | | | | Vesicular stomatitis Alagoas virus | VDSQFINGKC |
| | | | | Vesicular stomatitis Indiana virus | ooooo*o*oooo*o*o* |
| | | | | Vesicular stomatitis New Jersey virus | o*oooooooo |
| | Lyssavirus | | Aravan virus | | |
| | | | Australian bat | | |
| | | | lyssavirus | | |
| | | | Duvenhage virus | | |
| | | | European bat | | |
| | | | lyssavirus 1 | | |
| | | | European bat | | |
| | | | lyssavirus 2 | | |
| | | | Irkut virus | | |
| | | | Khujand virus | | |
| | | | Lagos bat virus | | |
| | | | Mokola virus | | |
| | | | West Caucasian | | |
| | | | bat virus | | |
| | | | Rabies virus | Rabies virus AB21 | SEQ ID NO: 5 |
| | | | | Rabies virus AB22 | GFTCTGVVTEAETYTNFVGYVT |
| | | | | Rabies virus AVO1 | *o****o**o*oo*oooo*** |
| | | | | Rabies virus BNG4 | SEQ ID NO: 6 |
| | | | | Rabies virus BNG5 | SLHNPYPDYRWLRTVKTT |
| | | | | Rabies virus China/DRV | *ooooooooooo***o* |
| | | | | Rabies virus China/MRV | SEQ ID NO: 210 |
| | | | | Rabies virus CVS-11 | ESLVIISPSVADLDPYDRSLHS |
| | | | | Rabies virus ERA | *ooo***oooo*o**ooo |
| | | | | Rabies virus Eth2003 | SEQ ID NO: 211 |
| | | | | Rabies virus HEP-FLURY | CKLKLCGVLGLRLMDGT |
| | | | | Rabies virus India | *ooo****oooo*ooo* |
| | | | | Rabies virus Nishigahara RCEH | SEQ ID NO: 212 |
| | | | | Rabies virus Ontario fox | ILGPDGNVLIPEMQSS |
| | | | | Rabies virus Ontario skunk | o**o*ooo*******o |
| | | | | Rabies virus PM | SEQ ID NO: 213 |
| | | | | Rabies virus red fox/08RS- | QHMELLESSVIPLVHPL |
| | | | | 1981/Udine/2008 | *ooo**o*ooo**oo** |
| | | | | Rabies virus SAD B19 | |
| | | | | Rabies virus silver-haired bat- | |
| | | | | associated SHBRV | |
| | | | | Rabies virus strain Pasteur vaccin | |
| | | | | Rabies virus strain Street | |
| | | | | Rabies virus vnukovo-32 | |
| | | | | Thailand genotype 1 dog lyssavirus | |
| | | | unclassified | Bokeloh bat lyssavirus | |
| | | | Lyssavirus | European bat lyssavirus | |
| | | | | Lyssavirus Ozernoe | |
| | | | | Shimoni bat virus | |
| | Novirhabdovirus | | Hirame | | |
| | | | rhabdovirus | | |
| | | | Infectious | | |
| | | | hematopoietic | | |
| | | | necrosis virus | | |
| | | | Snakehead | | |
| | | | rhabdovirus | | |
| | | | Viral hemorrhagic | | |
| | | | septicemia virus | | |
| | unassigned Rhabdoviridae | | Bangoran virus | | |
| | | | Bimbo virus | | |
| | | | Bivens Arm virus | | |
| | | | Flanders virus | | |
| | | | Garba virus | | |
| | | | Klamath virus | | |
| | | | Malpais Spring | | |
| | | | virus | | |
| | | | Nasoule virus | | |
| | | | Ngaingan virus | | |
| | | | Ouango virus | | |
| | | | Sigma virus | | |
| | | | Tupaia virus | | |
| | | | Wongabel virus | | |
| | | | | Peptides from domains from | knock-out (K.O.) |
| | | | | fusion proteins exhibiting | mutants of the |
| | | Species | Species | immunosuppressive activity | immunosuppressive |
| Family | Genus | (group) | (Strain) | (ISU) | domain (ISU) |
| Flavi- | Flavi-virus | Aroa virus | Bussuquara virus | | SEQ ID NO: 202 |
| viridae | | | guape virus | | DRGWGNGCGDFGKG |
| | | | Naranjal virus | | |
| | | Dengue virus | Dengue 1 | SEQ ID NO: 2 | |
| | | | | DRGWGNGCGLFGKG | |
| | | | | ************** | |
| | | | | SEQ ID NO: 172 | |
| | | | | KGSSIGKMFESTYRGAKRMAILG | |
| | | | Dengue 2 | SEQ ID NO: 2 | |
| | | | | DRGWGNGCGLFGKG | |
| | | | | ************** | |
| | | | Dengue 3 | SEQ ID NO: 2 | |
| | | | | DRGWGNGCGLFGKG | |
| | | | | ************** | |
| | | | | SEQ ID NO: 204 | |
| | | | | GDTAWDFGSVGGVLNSLGK | |
| | | | | ******************* | |
| | | | Dengue 4 | SEQ ID NO: 2 | |
| | | | | DRGWGNGCGLFGKG | |
| | | | | ************** | |
| | | Japanese | Japanese encephalitis virus | SEQ ID NO: 2 | |
| | | encephalitis | | DRGWGNGCGLFGKG | |
| | | virus group | | ************** | |
| | | | Koutango virus | SEQ ID NO: 2 | |
| | | | | DRGWGNGCGLFGKG | |
| | | | | ************** | |
| | | | Murray Valley encephalitis Virus | SEQ ID NO: 2 | |
| | | | | DRGWGNGCGLFGKG | |
| | | | | ************** | |
| | | | St. Louis encephalitis virus | SEQ ID NO: 2 | |
| | | | | DRGWGNGCGLFGKG | |
| | | | | ************** | |
| | | | Usutu virus | SEQ ID NO: 2 | |
| | | | | DRGWGNGCGLFGKG | |
| | | | | ************** | |
| | | | West Nile virus | SEQ ID NO: 2 | |
| | | | | DRGWGNGCGLFGKG | |
| | | | | ************** | |
| | | Kokobera virus | Kokobera Virus | SEQ ID NO: 2 | |
| | | group | unclassified Kokobera | DRGWGNGCGLFGKG | |
| | | | virus group | | |
| | | Modoc Virus group | Modoc Virus | | |
| | | | Cowbone Ridge virus | | |
| | | | Jutiapa virus | | |
| | | | Sal Vieja virus | | |
| | | | San Perlita virus | | |
| | | mosquito-borne | Ilheus virus | SEQ ID NO: 2 | |
| | | viruses | | DRGWGNGCGLFGKG | |
| | | | Sepik virus | SEQ ID NO: 2 | |
| | | | | DRGWGNGCGLFGKG | |
| | | Ntaya virus group | Bagaza virus | SEQ ID NO: 2 | |
| | | | | DRGWGNGCGLFGKG | |
| | | | Tembusu virus | SEQ ID NO: 2 | |
| | | | | DRGWGNGCGLFGKG | |
| | | | Yokose virus | SEQ ID NO: 2 | |
| | | | | DRGWGNGCGLFGKG | |
| | | Rio Bravo virus | Apoi virus | SEQ ID NO: 2 | |
| | | group | | DRGWGNGCGLFGKG | |
| | | | Entebbe bat virus | SEQ ID NO: 2 | |
| | | | | DRGWGNGCGLFGKG | |
| | | | Rio Bravo virus | SEQ ID NO: 2 | |
| | | | | DRGWGNGCGLFGKG | |
| | | | Saboya virus | | |
| | | Seaborne tick- | Meaban virus | | |
| | | borne virus group | | | |
| | | | Saumarez Reef virus | | |
| | | | Tyuleniy virus | | |
| | | Spondweni virus | Zika virus | SEQ ID NO: 2 | |
| | | group | | DRGWGNGCGLFGKG | |
| | | | Kyasanur forest disease virus | | |
| | | | Langat virus | | |
| | | | Louping ill virus | | |
| | | | Omsk hemorrhagic fever virus | | |
| | | | Powassan virus | | |
| | | | Royal Farm virus | | |
| | | | Tick-borne encephalitis virus | | |
| | | Yaounde virus | | SEQ ID NO: 2 | |
| | | | | DRGWGNGCGLFGKG | |
| | | Yellow fever | Banzi virus | SEQ ID NO: 2 | |
| | | virus group | | DRGWGNGCGLFGKG | |
| | | | Bouboui virus | SEQ ID NO: 2 | |
| | | | | DRGWGNGCGLFGKG | |
| | | | Edge Hill virus | | |
| | | | Uganda S virus | | |
| | | | Wesselsbron virus | | |
| | | | Yellow fever virus | SEQ ID NO: 2 | |
| | | | | DRGWGNGCGLFGKG | |
| | | unclassified | Batu Cave virus | SEQ ID NO: 2 | |
| | | Flavivirus | Cacipacore virus | DRGWGNGCGLFGKG | |
| | | | Calbertado virus | | |
| | | | Cell fusing agent virus | | |
| | | | Chaoyang virus | | |
| | | | Chimeric Tick-borne encephalitis | | |
| | | | virus/Dengue virus 4 | | |
| | | | Culex theileri flavivirus | | |
| | | | Donggang virus | | |
| | | | Duck hemorrhagic ovaritis virus | | |
| | | | Flavivirus Aedes/MO-Ac/ITA/2009 | | |
| | | | Flavivirus Anopheles/ | | |
| | | | PV-Am/ITA/2009 | | |
| | | | Flavivirus CbaAr4001 | | |
| | | | Flavivirus FSME | | |
| | | | Flavivirus Phlebotomine/76/ | | |
| | | | Arrabida/2007 | | |
| | | | Gadgets Gully virus | | |
| | | | Greek goat encephalitis virus | | |
| | | | Jugra virus | | |
| | | | Kadam virus | | |
| | | | Kamiti River virus | | |
| | | | Kedougou virus | | |
| | | | Montana myotis | | |
| | | | leukoencephalitis virus | | |
| | | | Mosquito flavivirus | | |
| | | | Ngoye virus | | |
| | | | Nounane virus | | |
| | | | Phlebotomus flavivirus Alg_F19 | | |
| | | | Phlebotomus flavivirus Alg_F8 | | |
| | | | Quang Binh virus | | |
| | | | Russian Spring-Summer | | |
| | | | encephalitis virus | | |
| | | | Sokoluk virus | | |
| | | | Spanish sheep encephalitis virus | | |
| | | | T′Ho virus | | |
| | | | Tai forest virus B31 | | |
| | | | Tamana bat virus | | |
| | | | Tick-borne flavivirus | | |
| | | | Wang Thong virus | | |
| | | | Flavivirus sp. | | |
| | | | Aedes flavivirus | | |
| | | | Culex flavivirus | | |
| | Hepacivirus | Hepatitis C | Hepatitis C virus genotype 1 a | SEQ ID NO: 3 | |
| | | virus | | GLIHLHQNIVDVQYLYG | |
| | | | | SEQ ID NO: 175 | |
| | | | | PALSTGLIHLHQNIVDVQ | |
| | | | Hepatitis C virus genotype 1b | | |
| | | | Hepatitis C virus genotype 2 | | |
| | | | Hepatitis C virus genotype 3 | SEQ ID NO: 3 | |
| | | | | GLIHLHQNIVDVQYLYG | |
| | | | Hepatitis C virus genotype 4 | SEQ ID NO: 3 | |
| | | | | GLIHLHQNIVDVQYLYG | |
| | | | Hepatitis C virus genotype 5 | | |
| | | | Hepatitis C virus genotype 6 | SEQ ID NO: 3 | |
| | | | | GLIHLHQNIVDVQYLYG | |
| | | | All Hepatitis C virus | SEQ ID NO: 3 | |
| | | | | GLIHLHQNIVDVQYLYG | |
| | Pesti virus | Border disease | Border disease virus - | | |
| | | virus | Border disease virus - X818 | | |
| | | | Border disease virus 1 | | |
| | | | Border disease virus 2 | | |
| | | | Border disease virus 3 | | |
| | | | Border disease virus isolates | | |
| | | Bovine viral | Bovine viral diarrhea virus 1-CP7 | | |
| | | diarrhea | Bovine viral diarrhea virus 1-NADL | | |
| | | virus 1 | Bovine viral diarrhea | | |
| | | | virus 1-Osloss | | |
| | | | Bovine viral diarrhea virus 1-SD1 | | |
| | | | Bovine viral diarrhea virus | | |
| | | | isolates and strains | | |
| | | | Bovine viral diarrhea virus type 1a | | |
| | | | Bovine viral diarrhea virus type 1b | | |
| | | | Pestivirus isolate 97-360 | | |
| | | | Pestivirus isolate Hay 87/2210 | | |
| | | | Pestivirus strain mousedeer | | |
| | | | Pestivirus type 1 isolates | | |
| | | Bovine viral | Bovine viral diarrhea virus 2 | | |
| | | diarrhea virus 2 | Pestivirus sp. strain 178003 | | |
| | | (BVDV-2) | Pestivirus sp. strain 5250Giessen-3 | | |
| | | | Bovine viral diarrhea virus-2 isolate | | |
| | | | SCP | | |
| | | Classical swine | Classical swine fever virus | | |
| | | fever virus | Hog cholera virus strain Zoelen | | |
| | | unclassified | Bovine viral diarrhea virus 3 | | |
| | | Pestivirus | | | |
| | | | Chamois pestivirus 1 | | |
| | | | Porcine pestivirus isolate Bungowannah | | |
| | Unnclassified | | Barkedji virus | | |
| | Flaviviridae | | Canine hepacivirus AAK-2011 | | |
| | | | GB virus A | | |
| | | | Douroucouli hepatitis GB virus A | | |
| | | | GBV-A-like agents | | |
| | | | GB virus D | | |
| | | | GBV-C/HGV group | | |
| | | | GB virus C | | |
| | | | Hepatitis GB virus C-like virus | | |
| | | | Hepatitis GB virus B | | |
| | | | Lammi virus | | |
| | | | Marmoset hepatitis GB virus A | | |
| | | | Nakiwogo virus | | |
| | | | Turkey meningoencephalitis virus | | |
| Toga- | Alpha-virus | Aura virus | | | |
| viridae | | Barmah Forest | | | |
| | | virus | | | |
| | | Middelburg virus | | | |
| | | Ndumu virus | | | |
| | | Salmon pancreas | | | |
| | | disease virus | | | |
| | | Getah virus | | | |
| | | Mayaro virus | | | |
| | | Trocara virus | | | |
| | | EEEV complex | | | |
| | | WEEV complex | Fort Morgan virus | | |
| | | | Highlands J virus | | |
| | | | Sindbis virus | | |
| | | | Western equine encephalomyelitis virus | | |
| | | | Whataroa virus | | |
| | | VEEV complex | Cabassou virus | | |
| | | | Mucambo virus | | |
| | | | Pixuna virus | | |
| | | | Venezuelan equine encephalitis virus | | |
| | | SFV complex | Bebaru virus | | |
| | | | O'nyong-nyong virus | | |
| | | | Ross River virus | | |
| | | | Semliki forest virus | | |
| | | | Una virus | | |
| | | | Chikungunya virus | | |
| | Rubivirus | Rubella virus | Rubella virus (strain BRD1) | | |
| | | | Rubella virus (strain BRDII) | | |
| | | | Rubella virus (strain Cendehill) | | |
| | | | Rubella virus (strain M33) | | |
| | | | Rubella virus (strain RN-UK86) | | |
| | | | Rubella virus (strain THERIEN) | | |
| | | | Rubella virus (strain TO-336 vaccine) | | |
| | | | Rubella virus (strain TO-336) | | |
| | | | Rubella virus (vaccine strain RA27/3) | | |
| Bunya- | Hanta-virus | Amur virus | | | |
| viridae | (continued on | Bayou virus | | | |
| | next page) | Black Creek Canal | | | |
| | | virus | | | |
| | | Cano Delgadito | | | |
| | | virus | | | |
| | | Calabazo virus | | | |
| | | Catacamas virus | | | |
| | | Choclo virus | | | |
| | | Dobrava-Belgrade | | | |
| | | virus | | | |
| | | El Moro Canyon | | | |
| | | virus | | | |
| | | Hantaan virus | | | |
| | | Isla Vista virus | | | |
| | | Khabarovsk virus | | | |
| | | Laguna Negra | | | |
| | | virus | | | |
| | | Limestone Canyon | | | |
| | | virus | | | |
| | | Monongahela virus | | | |
| | | Muleshoe virus | | | |
| | | Muju virus | | | |
| | | New York virus | | | |
| | | Oran virus | | | |
| | | Playa de Oro | | | |
| | | virus | | | |
| | | Prospect Hill | | | |
| | | virus | | | |
| | | Puumala virus | | | |
| | | Rio Mamore virus | | | |
| | | Rio Segundo virus | | | |
| | | Saaremaa virus | | | |
| | | Seoul virus | | | |
| | | Sin Nombre virus | | | |
| | | Soochong virus | | | |
| | | Thailand virus | | | |
| | | Thottapalayam | | | |
| | | virus | | | |
| | | Topografov virus | | | |
| | | Tula virus | | | |
| | Ortho-bunya-virus | Anopheles A virus | | | |
| | | Anopheles B virus | | | |
| | | Bakau virus | | | |
| | | Batama virus | | | |
| | | Bwamba virus | | | |
| | | Caraparu virus | | | |
| | | Kaeng Khoi virus | | | |
| | | Kairi virus | | | |
| | | Madrid virus | | | |
| | | Main Drain virus | | | |
| | | Marituba virus | | | |
| | | Nyando virus | | | |
| | | Oriboca virus | | | |
| | | Oropouche virus | | | |
| | | Sathuperi virus | | | |
| | | Shamonda virus | | | |
| | | Shuni virus | | | |
| | | Simbu virus | | | |
| | | Tacaiuma virus | | | |
| | | Tete virus | | | |
| | | Turlock virus | | | |
| | | unclassified | | | |
| | | Orthobunyavirus | | | |
| | | Akabane virus | Sabo virus | | |
| | | | Tinaroo virus | | |
| | | | Yaba-7 virus | | |
| | | Bunyamwera virus | Batai virus | | |
| | | | Birao virus | | |
| | | | Bozo virus | | |
| | | | Cache Valley virus | | |
| | | | Fort Sherman virus | | |
| | | | Germiston virus | | |
| | | | Guaroa virus | | |
| | | | Iaco virus | | |
| | | | Ilesha virus | | |
| | | | Lokern virus | | |
| | | | Maguari virus | | |
| | | | Mboke virus | | |
| | | | Ngari virus | | |
| | | | Northway virus | | |
| | | | Playas virus | | |
| | | | Potosi virus | | |
| | | | Shokwe virus | | |
| | | | Tensaw virus | | |
| | | | Tlacotalpan virus | | |
| | | | Xingu virus | | |
| | | California | California encephalitis | | |
| | | Encephalitis | serogroup virus | | |
| | | virus | LEIV | | |
| | | | California encephalitis virus- | | |
| | | | BFS-283 | | |
| | | | Chatanga virus | | |
| | | | Inkoo virus | | |
| | | | Jamestown Canyon virus | | |
| | | | Jamestown Canyon-like virus | | |
| | | | Jerry Slough virus | | |
| | | | Keystone virus | | |
| | | | La Crosse virus | | |
| | | | Lumbo virus | | |
| | | | Melao virus | | |
| | | | Morro Bay virus | | |
| | | | San Angelo virus | | |
| | | | Serra do Navio virus | | |
| | | | Snowshoe hare virus | | |
| | | | South River virus | | |
| | | | Tahyna virus | | |
| | | | Trivittatus virus | | |
| | | Caraparu virus | Apeu virus | | |
| | | | Bruconha virus | | |
| | | | Ossa virus | | |
| | | | Vinces virus | | |
| | | Manzanilla virus | Buttonwillow virus | | |
| | | | Ingwavuma virus | | |
| | | | Mermet virus | | |
| | | Marituba virus | Gumbo Limbo virus | | |
| | | | Murutucu virus | | |
| | | | Nepuyo virus | | |
| | | | Restan virus | | |
| | | Wyeomyia virus | Anhembi virus | | |
| | | | BeAr328208 virus | | |
| | | | Macaua virus | | |
| | | | Sororoca virus | | |
| | | | Taiassui virus | | |
| | Phlebovirus | Bujaru virus | | | |
| | | Candiruvirus | | | |
| | | Chilibre virus | | | |
| | | Frijoles virus | | | |
| | | Punta | | | |
| | | Tor_\|Salehabad | | | |
| | | virus | | | |
| | | Sandflyfever | | | |
| | | Naples virus | | | |
| | | Uukuniemi viruso | | | |
| | | virus | | | |
| | | Rift Valley | | | |
| | | fever virus | | | |
| | | unclassified | Anhanga virus | | |
| | | Phlebovirus | Arumowot virus | | |
| | | (continued on | Chagres virus | | |
| | | next page) | Corfou virus | | |
| | | | Gabek Forest virus | | |
| | | | Itaporanga virus | | |
| | | | Phlebovirus Adria/ALB1/2005 | | |
| | | | Phlebovirus Adria/ALB5/2005 | | |
| | | | Phlebovirus AH12 | | |
| | | | Phlebovirus AH12/China/2010 | | |
| | | | Phlebovirus AH15/China/2010 | | |
| | | | Phlebovirus B105-05 | | |
| | | | Phlebovirus B151-04 | | |
| | | | Phlebovirus B43-02 | | |
| | | | Phlebovirus B68-03 | | |
| | | | Phlebovirus B79-02 | | |
| | | | Phlebovirus Chios-A | | |
| | | | Phlebovirus Cyprus | | |
| | | | Phlebovirus HB29/China/2010 | | |
| | | | Phlebovirus HN13/China/2010 | | |
| | | | Phlebovirus HN6/China/2010 | | |
| | | | Phlebovirus Hu/Xinyang1/China/2010 | | |
| | | | Phlebovirus Hu/Xinyang2/China/2010 | | |
| | | | Phlebovirus IB13-04 | | |
| | | | Phlebovirus JS2007-01 | | |
| | | | Phlebovirus JS24 | | |
| | | | Phlebovirus JS26 | | |
| | | | Phlebovirus JS3/China/2010 | | |
| | | | Phlebovirus JS4/China/2010 | | |
| | | | Phlebovirus JS6 | | |
| | | | Phlebovirus JSD1 | | |
| | | | Phlebovirus LN2/China/2010 | | |
| | | | Phlebovirus LN3/China/2010 | | |
| | | | Phlebovirus sandflies/Gr29/Spain/2004 | | |
| | | | Phlebovirus sandflies/Gr36/Spain/2004 | | |
| | | | Phlebovirus sandflies/Gr44/Spain/2004 | | |
| | | | Phlebovirus sandflies/Gr49/Spain/2004 | | |
| | | | Phlebovirus sandflies/Gr52/Spain/2004 | | |
| | | | Phlebovirus sandflies/Gr65/Spain/2004 | | |
| | | | Phlebovirus sandflies/Gr98/Spain/2004 | | |
| | | | Phlebovirus SD24/China/2010 | | |
| | | | Phlebovirus SD4/China/2010 | | |
| | | | Phlebovirus tick/XCQ-2011 | | |
| | | | Phlebovirus XLL/China/2009 | | |
| | | | Rio Grande virus | | |
| | | | Salobo virus | | |
| | | | Sandfly fever sicilian virus | | |
| | | | Sandfly Sicilian Turkey virus | | |
| | | | Utique virus | | |
| | | | Phlebovirus sp. | | |
| | | | Phlebovirus sp. Be An 24262 | | |
| | | | Phlebovirus sp. Be An 356637 | | |
| | | | Phlebovirus sp. Be An 416992 | | |
| | | | Phlebovirus sp. Be An 578142 | | |
| | | | Phlebovirus sp. Be Ar 371637 | | |
| | | | Phlebovirus sp. Co Ar 170255 | | |
| | | | Phlebovirus sp. Co Ar 171616 | | |
| | | | Phlebovirus sp. GML 902878 | | |
| | | | Phlebovirus sp. Pa Ar 2381 | | |
| | | | Phlebovirus sp. PAN 479603 | | |
| | | | Phlebovirus sp. PAN 483391 | | |
| | | | Phlebovirus sp. VP-161A | | |
| | | | Phlebovirus sp. VP-334K | | |
| | | | Phlebovirus sp. VP-366G | | |
| Ortho- | Influenzavirus A | Influenza A | INFA H1 | | INF F#2 DELTA6: |
| myxo- | | virus | | | SEQ ID NO: 201 |
| viridae | | | | | GLFGAAGFIENGWEG |
| | | | | | InFAH1-3: SEQ ID NO: 203 |
| | | | INFA H2 | | |
| | | | INFA H3 | SEQ ID NO: 4 | |
| | | | | GLFGAIAGFIENGWEG | |
| | | | INFA H4 | | |
| | | | INFA H5 | | |
| | | | INFA H6 | | |
| | | | INFA H7 | SEQ ID NO: 4 | |
| | | | | GLFGAIAGFIENGWEG | |
| | | | INFA H8 | | |
| | | | INFA H9 | | |
| | | | INFA H10 | SEQ ID NO: 4 | |
| | | | | GLFGAIAGFIENGWEG | |
| | | | INFA H11 | | |
| | | | INFA H12 | | |
| | | | INFA H13 | | |
| | | | INFA H14 | | |
| | | | INFA H15 | SEQ ID NO: 4 | |
| | | | | GLFGAIAGFIENGWEG | |
| | | | INFA H16 | | |
| | Influenzavirus B | Influenza B virus | All strains | | |
| | Influenzavirus C | Influenza C virus | | | |
| Paramyxo- | Paramyxovirinae | Avulavirus | Avian paramyxovirus 2 Yucaipa virus | | |
| viridae | | | Avian paramyxovirus 3 | | |
| | | | Avian paramyxovirus 3b | | |
| | | | Avian paramyxovirus 4 | | |
| | | | Avian paramyxovirus 5 | | |
| | | | Avian paramyxovirus 6 | | |
| | | | Avian paramyxovirus 7 | | |
| | | | Avian paramyxovirus 8 | | |
| | | | Avian paramyxovirus 9 | | |
| | | | Newcastle disease virus | | |
| | | | Pigeon paramyxovirus 1 | | |
| | | | unclassified Avulavirus | | |
| | | | Avian paramyxovirus 10_Avian | | |
| | | | paramyxovirus duck/Miyagi/885/05 | | |
| | | | Avian paramyxovirus penguin/Falkland | | |
| | | | Islands/324/2007 | | |
| | | | Goosramyxovirus HZ | | |
| | | | Goose paramyxovirus JS/1/97/Go | | |
| | | | Goose paramyxovirus SF02 | | |
| | | Henipavirus | Hendra virus Hendra virus | | |
| | | | horse/Australia/Hendra/1994 | | |
| | | | Nipah virus | | |
| | | | unclassified Henipavirus | | |
| | | | Bat paramyxovirus | | |
| | | | Eid.hel/GH45/2008 | | |
| | | Morbillivirus | Canine distemper virus | | |
| | | | Cetacean morbillivirus_Dolphin | | |
| | | | morbillivirus_Pilot whale morbillivirus | | |
| | | | Porpoise morbillivirus | | |
| | | | Measles virus | | |
| | | | Peste-des-petits-ruminants virus | | |
| | | | Phocine distemper virus | | |
| | | | Phocine distemper virus 1 | | |
| | | | Phocine distemper virus-2 | | |
| | | | Rinderpest virus | | |
| | | Respirovirus | Bovine parainfluenza virus 3 | | |
| | | | Porcine paramyxovirus strain Frost | | |
| | | | Porcine paramyxovirus strain Texas | | |
| | | | Human parainfluenza virus 1 | | |
| | | | Human parainfluenza virus 3 | | |
| | | | Simian Agent 10 | | |
| | | | Sendai virus | | |
| | | | unclassified Respirovirus | | |
| | | | Atlantic salmon respirovirus | | |
| | | | Guinea pig parainfluenza virus TS-9 | | |
| | | | Pacific salmon paramyxovirus | | |
| | | | Trask River 1983 Swine parainfluenza | | |
| | | | virus 3 | | |
| | | | Tursiops truncatus parainfluenza virus 1 | | |
| | | Rubulavirus | Human parainfluenza virus 2 | | |
| | | | Human parainfluenza virus 2 (strain | | |
| | | | Greer) | | |
| | | | Human parainfluenza virus 2 (strain | | |
| | | | Toshiba) | | |
| | | | Human parainfluenza virus 4 | | |
| | | | Human parainfluenza virus 4a | | |
| | | | Human parainfluenza virus 4b | | |
| | | | Mapuera virus | | |
| | | | Mumps virus | | |
| | | | Parainfluenza virus 5 | | |
| | | | Porcine rubulavirus | | |
| | | | Simian virus 41 | | |
| | | | unclassified Rubulavirus | | |
| | | | Porcine parainfluenza virus | | |
| | | | Tuhoko virus 1 | | |
| | | | Tuhoko virus 2 | | |
| | | | Tuhoko virus 3 | | |
| | | unclassified | Atlantic salmon paramyxovirus | | |
| | | Paramyxovirinae | Beilong virus | | |
| | | | Canine parainfluenza virus | | |
| | | | Chimeric human parainfluenza virus | | |
| | | | rPIV3-2 | | |
| | | | Fer-de-lance virus | | |
| | | | J-virus | | |
| | | | Menangle virus | | |
| | | | Mossman virus | | |
| | | | Murayama virus | | |
| | | | Ovine parainfluenza virus 3 | | |
| | | | Pacific salmon paramyxovirus | | |
| | | | Paramyxovirus GonoGER85 | | |
| | | | Recombinant PIV3/PIV1 virus | | |
| | | | Reptilian paramyxovirus | | |
| | | | Salem virus | | |
| | | | Salmo salar paramyxovirus | | |
| | | | Snake ATCC-VR-1408 paramyxovirus | | |
| | | | Snake ATCC-VR-1409 paramyxovirus | | |
| | | | Tioman virus | | |
| | | | Tupaia paramyxovirus | | |
| | Pneumovirus | Human respiratory | Human respiratory syncytial virus A | | |
| | | syncytial virus | Human respiratory syncytial virus | | |
| | | | (strain RSB1734) | | |
| | | | Human respiratory syncytial virus | | |
| | | | (strain RSB5857) | | |
| | | | Human respiratory syncytial virus | | |
| | | | (strain RSB6190) | | |
| | | | Human respiratory syncytial virus | | |
| | | | (strain RSB6256) | | |
| | | | Human respiratory syncytial virus | | |
| | | | (strain RSB642) | | |
| | | | Human respiratory syncytial virus | | |
| | | | (strain RSB6614) | | |
| | | | Human respiratory syncytial virus A | | |
| | | | strain Long LinkOut | | |
| | | | Human respiratory syncytial virus A2 | | |
| | | | Human respiratory syncytial virus B | | |
| | | | Human respiratory syncytial | | |
| | | | virus (subgroup B/strain 18537) | | |
| | | | Human respiratory syncytial virus | | |
| | | | (subgroup B strain 8/60) | | |
| | | | Human Respiratory syncytial virus 9320 | | |
| | | | Human respiratory syncytial virus B1 | | |
| | | | Human respiratory syncytial virus S2 | | |
| | | | Human respiratory syncytial virus strain | | |
| | | | RSS-2 | | |
| | | | unclassified Human respiratory syncytial | | |
| | | | virus | | |
| | | Bovine | All strains | | |
| | | respiratory | | | |
| | | syncytial virus | | | |
| | Metapneum | Avian metapneumo- | All strains | | |
| | | virus | | | |
| | | Human metapneumo- | All strains | | |
| | | virus | | | |
| Corona- | Coronavirinae | Alphacorona-virus | Alphacoronavirus 1 | | |
| viridae | | | Coronavirus group 1b | | |
| | | | Human coronavirus 229E | | |
| | | | Human coronavirus NL63 | | |
| | | | Miniopterus bat coronavirus 1 | | |
| | | | Miniopterus bat coronavirus HKU8 | | |
| | | | Porcine epidemic diarrhea virus | | |
| | | | Rhinolophus bat coronavirus HKU2 | | |
| | | | Scotophilus bat coronavirus 512 | | |
| | | | unclassified Alphacoronavirus | | |
| | | Betacorona- | Betacoronavirus 1 | | |
| | | virus | Coronavirus group 2b | | |
| | | | Coronavirus group 2c | | |
| | | | Human coronavirus HKU1 | | |
| | | | Murine coronavirus | | |
| | | | Pipistrellus bat coronavirus HKU5 | | |
| | | | Rousettus bat coronavirus HKU9 | | |
| | | | Severe acute respiratory syndrome- | | |
| | | | related coronavirus recombinant SARSr- | | |
| | | | CoV | | |
| | | | SARS coronavirus | | |
| | | | Tylonycteris bat coronavirus HKU4 | | |
| | | | unclassified Betacoronavirus | | |
| | | Gammacorona- | Avian coronavirus | | |
| | | virus | Beluga Whale coronavirus SW1 | | |
| | | unclassified | Alpaca coronavirus CA08-1/2008 | | |
| | | coronaviruses | Bat coronavirus | | |
| | | | Bird droppings coronavirus | | |
| | | | Bovine respiratory coronavirus | | |
| | | | Chicken enteric coronavirus | | |
| | | | Coronavirus Anas | | |
| | | | Coronavirus oystercatcher/p17/2006/GBR | | |
| | | | Coronavirus red knot/p60/2006/GBR | | |
| | | | Ferret enteric coronavirus 1202 | | |
| | | | Ferret systemic coronavirus MSU-S | | |
| | | | Ferret systemic coronavirus WADL | | |
| | | | Guangxi coronaviridae | | |
| | | | Human coronavirus NO | | |
| | | | Human enteric coronavirus strain 4408 | | |
| | | | Kenya bat coronavirus | | |
| | | | Mink coronavirus strain WD1133 | | |
| | | | Parrot coronavirus AV71/99 | | |
| | | | Quail coronavirus Italy/Elvia/2005 | | |
| | | | Tai Forest coronavirus | | |
| | | | unidentified coronavirus | | |
| | | | unidentified human coronavirus | | |
| Arena- | Arena-virus | LCMV-Lassa virus | Ippy virus | | |
| viridae | | (Old World) | Lassa virus | | |
| | | complex | Lujo virus | | |
| | | | Lymphocytic choriomeningitis virus | | |
| | | | Mobala virus | | |
| | | | Mopeia virus | | |
| | | Tacaribe virus | Amapari virus | | |
| | | (New World) | Chapare virus | | |
| | | complex | Flexal virus | | |
| | | | Guanarito virus | | |
| | | | Junin virus | | |
| | | | Latino virus | | |
| | | | Machupo virus | | |
| | | | Oliveros virus | | |
| | | | Parana virus | | |
| | | | Pichinde virus | | |
| | | | Pirital virus | | |
| | | | Sabia virus | | |
| | | | Tacaribe virus | | |
| | | | Tamiami virus | | |
| | | | Whitewater Arroyo virus | | |
| Hepadna- | Genus | Hepatitis B virus | HBV genotype A | | |
| viridae | | Orthohepadnavirus | HBV genotype B | | |
| | | | HBV genotype C | | |
| | | | HBV genotype D | | |
| | | | HBV genotype E | | |
| | | | HBV genotype F | | |
| | | | HBV genotype G | | |
| | | | HBV genotype H | | |
| | | | Hepatitis B virus alpha1 | | |
| | | | Hepatitis B virus LSH/chimpanzee | | |
| | | | Hepatitis B virus strain cpz | | |
| | | | Hepatitis B virus subtype adr | | |
| | | | Hepatitis B virus subtype adw | | |
| | | | Hepatitis B virus subtype adyw | | |
| | | | Hepatitis B virus subtype ayw | | |
| Rhabdo- | Dimarhabdovirus | Ephemerovirus | Bovine ephemeral fever virus | | |
| viridae | | Vesiculovirus | Carajas virus | | |
| | | | Chandipura virus | | |
| | | | Cocal virus | | |
| | | | Isfahan virus | | |
| | | | Maraba virus | | |
| | | | Piry virus | | |
| | | | recombinant Vesiculovirus | | |
| | | | Spring viraemia of carp virus | | |
| | | | Vesicular stomatitis Alagoas virus | | |
| | | | Vesicular stomatitis Indiana virus | | |
| | | | Vesicular stomatitis New Jersey virus | | |
| | Lyssavirus | Aravan virus | | | |
| | | Australian bat | | | |
| | | lyssavirus | | | |
| | | Duvenhage virus | | | |
| | | European bat | | | |
| | | lyssavirus 1 | | | |
| | | European bat | | | |
| | | lyssavirus 2 | | | |
| | | Irkut virus | | | |
| | | Khujand virus | | | |
| | | Lagos bat virus | | | |
| | | Mokola virus | | | |
| | | West Caucasian | | | |
| | | bat virus | | | |
| | | Rabies virus | Rabies virus AB21 | | |
| | | | Rabies virus AB22 | | |
| | | | Rabies virus AVO1 | | |
| | | | Rabies virus BNG4 | | |
| | | | Rabies virus BNG5 | | |
| | | | Rabies virus China/DRV | | |
| | | | Rabies virus China/MRV | | |
| | | | Rabies virus CVS-11 | | |
| | | | Rabies virus ERA | | |
| | | | Rabies virus Eth2003 | | |
| | | | Rabies virus HEP-FLURY | | |
| | | | Rabies virus India | | |
| | | | Rabies virus Nishigahara RCEH | | |
| | | | Rabies virus Ontario fox | | |
| | | | Rabies virus Ontario skunk | | |
| | | | Rabies virus PM | | |
| | | | Rabies virus red fox/08RS- | | |
| | | | 1981/Udine/2008 | | |
| | | | Rabies virus SAD B19 | | |
| | | | Rabies virus silver-haired bat- | | |
| | | | associated SHBRV | | |
| | | | Rabies virus strain Pasteur vaccin | | |
| | | | Rabies virus strain Street | | |
| | | | Rabies virus vnukovo-32 | | |
| | | | Thailand genotype 1 dog lyssavirus | | |
| | | unclassified | Bokeloh bat lyssavirus | | |
| | | Lyssavirus | European bat lyssavirus | | |
| | | | Lyssavirus Ozernoe | | |
| | | | Shimoni bat virus | | |
| | Novirhabdo- | Hirame | | | |
| | virus | rhabdovirus | | | |
| | | Infectious | | | |
| | | hematopoietic | | | |
| | | necrosis virus | | | |
| | | Snakehead | | | |
| | | rhabdovirus | | | |
| | | Viral hemorrhagic | | | |
| | | septicemia virus | | | |
| | unassigned | Bangoran virus | | | |
| | Rhabdoviridae | Bimbo virus | | | |
| | | Bivens Arm virus | | | |
| | | Flanders virus | | | |
| | | Garba virus | | | |
| | | Klamath virus | | | |
| | | Malpais Spring | | | |
| | | virus | | | |
| | | Nasoule virus | | | |
| | | Ngaingan virus | | | |
| | | Ouango virus | | | |
| | | Sigma virus | | | |
| | | Tupaia virus | | | |
| | | Wongabel virus | | | |
| | | | | Name of | |
| | | Species | Species | envelope attachment/ | IU group and |
| Family | Genus | (group) | (Strain) | fusion protein | fusion type |
| Flavi- | Flavi-virus | Aroa virus | Bussuquara virus | Envelope protein prME | Group 1 Type II |
| viridae | | | guape virus | Fusion protein E | Fusion mechanism |
| | | | Naranjal virus | | |
| | | Dengue virus | Dengue 1 | | |
| | | | Dengue 2 | | |
| | | | Dengue 3 | | |
| | | | Dengue 4 | | |
| | | Japanese | Japanese encephalitis virus | | |
| | | encephalitis | | | |
| | | virus group | | | |
| | | | Koutango virus | | |
| | | | Murray Valley encephalitis Virus | | |
| | | | St. Louis encephalitis virus | | |
| | | | Usutu virus | | |
| | | | West Nile virus | | |
| | | Kokobera virus | Kokobera Virus unclassified Kokobera | | |
| | | group | virus group | | |
| | | Modoc Virus group | Modoc Virus | | |
| | | | Cowbone Ridge virus | | |
| | | | Jutiapa virus | | |
| | | | Sal Vieja virus | | |
| | | | San Perlita virus | | |
| | | mosquito-borne | Ilheus virus | | |
| | | viruses | | | |
| | | | Sepik virus | | |
| | | Ntaya virus group | Bagaza virus | | |
| | | | Tembusu virus | | |
| | | | Yokose virus | | |
| | | Rio Bravo virus | Apoi virus | | |
| | | group | | | |
| | | | Entebbe bat virus | | |
| | | | Rio Bravo virus | | |
| | | | Saboya virus | | |
| | | Seaborne tick- | Meaban virus | | |
| | | borne virus group | | | |
| | | | Saumarez Reef virus | | |
| | | | Tyuleniy virus | | |
| | | Spondweni virus | Zika virus | | |
| | | group | | | |
| | | | Kyasanur forest disease virus | | |
| | | | Langat virus | | |
| | | | Louping ill virus | | |
| | | | Omsk hemorrhagic fever virus | | |
| | | | Powassan virus | | |
| | | | Royal Farm virus | | |
| | | | Tick-borne encephalitis virus | | |
| | | Yaounde virus | | | |
| | | Yellow fever | Banzi virus | | |
| | | virus group | | | |
| | | | Bouboui virus | | |
| | | | Edge Hill virus | | |
| | | | Uganda S virus | | |
| | | | Wesselsbron virus | | |
| | | | Yellow fever virus | | |
| | | unclassified | Batu Cave virus | | |
| | | Flavivirus | Cacipacore virus | | |
| | | | Calbertado virus | | |
| | | | Cell fusing agent virus | | |
| | | | Chaoyang virus | | |
| | | | Chimeric Tick-borne encephalitis | | |
| | | | virus/Dengue virus 4 | | |
| | | | Culex theileri flavivirus | | |
| | | | Donggang virus | | |
| | | | Duck hemorrhagic ovaritis virus | | |
| | | | Flavivirus Aedes/MO-Ac/ITA/2009 | | |
| | | | Flavivirus Anopheles/PV-Am/ITA/2009 | | |
| | | | Flavivirus CbaAr4001 | | |
| | | | Flavivirus FSME | | |
| | | | Flavivirus Phlebotomine/76/Arrabida/2007 | | |
| | | | Gadgets Gully virus | | |
| | | | Greek goat encephalitis virus | | |
| | | | Jugra virus | | |
| | | | Kadam virus | | |
| | | | Kamiti River virus | | |
| | | | Kedougou virus | | |
| | | | Montana myotis leukoencephalitis virus | | |
| | | | Mosquito flavivirus | | |
| | | | Ngoye virus | | |
| | | | Nounane virus | | |
| | | | Phlebotomus flavivirus Alg_F19 | | |
| | | | Phlebotomus flavivirus Alg_F8 | | |
| | | | Quang Binh virus | | |
| | | | Russian Spring-Summer encephalitis virus | | |
| | | | Sokoluk virus | | |
| | | | Spanish sheep encephalitis virus | | |
| | | | T′Ho virus | | |
| | | | Tai forest virus B31 | | |
| | | | Tamana bat virus | | |
| | | | Tick-borne flavivirus | | |
| | | | Wang Thong virus | | |
| | | | Flavivirus sp. | | |
| | | | Aedes flavivirus | | |
| | | | Culex flavivirus | | |
| | Hepacivirus | Hepatitis C virus | Hepatitis C virus genotype 1 a | E1/E2 | |
| | | | Hepatitis C virus genotype 1b | | |
| | | | Hepatitis C virus genotype 2 | | |
| | | | Hepatitis C virus genotype 3 | | |
| | | | Hepatitis C virus genotype 4 | | |
| | | | Hepatitis C virus genotype 5 | | |
| | | | Hepatitis C virus genotype 6 | | |
| | | | All Hepatitis C virus | | |
| | Pesti virus | Border disease | Border disease virus - | E1/E2 | |
| | | virus | Border disease virus - X818 | | |
| | | | Border disease virus 1 | | |
| | | | Border disease virus 2 | | |
| | | | Border disease virus 3 | | |
| | | | Border disease virus isolates | | |
| | | Bovine viral | Bovine viral diarrhea virus 1-CP7 | | |
| | | diarrhea virus 1 | Bovine viral diarrhea virus 1-NADL | | |
| | | | Bovine viral diarrhea virus 1-Osloss | | |
| | | | Bovine viral diarrhea virus 1-SD1 | | |
| | | | Bovine viral diarrhea virus isolates and | | |
| | | | strains | | |
| | | | Bovine viral diarrhea virus type 1a | | |
| | | | Bovine viral diarrhea virus type 1b | | |
| | | | Pestivirus isolate 97-360 | | |
| | | | Pestivirus isolate Hay 87/2210 | | |
| | | | Pestivirus strain mousedeer | | |
| | | | Pestivirus type 1 isolates | | |
| | | Bovine viral | Bovine viral diarrhea virus 2 | | |
| | | diarrhea virus 2 | Pestivirus sp. strain 178003 | | |
| | | (BVDV-2) | Pestivirus sp. strain 5250Giessen-3 | | |
| | | | Bovine viral diarrhea virus-2 isolate | | |
| | | | SCP | | |
| | | Classical swine | Classical swine fever virus | | |
| | | fever virus | Hog cholera virus strain Zoelen | | |
| | | unclassified | Bovine viral diarrhea virus 3 | | |
| | | Pestivirus | | | |
| | | | Chamois pestivirus 1 | | |
| | | | Porcine pestivirus isolate Bungowannah | | |
| | Unnclassified | | Barkedji virus | | |
| | Flaviviridae | | Canine hepacivirus AAK-2011 | | |
| | | | GB virus A | | |
| | | | Douroucouli hepatitis GB virus A | | |
| | | | GBV-A-like agents | | |
| | | | GB virus D | | |
| | | | GBV-C/HGV group | | |
| | | | GB virus C | | |
| | | | Hepatitis GB virus C-like virus | | |
| | | | Hepatitis GB virus B | | |
| | | | Lammi virus | | |
| | | | Marmoset hepatitis GB virus A | | |
| | | | Nakiwogo virus | | |
| | | | Turkey meningoencephalitis virus | | |
| Toga- | Alpha-virus | Aura virus | | E2/E1 | |
| viridae | | Barmah Forest | | | |
| | | virus | | | |
| | | Middelburg virus | | | |
| | | Ndumu virus | | | |
| | | Salmon pancreas | | | |
| | | disease virus | | | |
| | | Getah virus | | | |
| | | Mayaro virus | | | |
| | | Trocara virus | | | |
| | | EEEV complex | | | |
| | | WEEV complex | Fort Morgan virus | | |
| | | | Highlands J virus | | |
| | | | Sindbis virus | | |
| | | | Western equine encephalomyelitis virus | | |
| | | | Whataroa virus | | |
| | | VEEV complex | Cabassou virus | | |
| | | | Mucambo virus | | |
| | | | Pixuna virus | | |
| | | | Venezuelan equine encephalitis virus | | |
| | | SFV complex | Bebaru virus | | |
| | | | O'nyong-nyong virus | | |
| | | | Ross River virus | | |
| | | | Semliki forest virus | | |
| | | | Una virus | | |
| | | | Chikungunya virus | | |
| | Rubivirus | Rubella virus | Rubella virus (strain BRD1) | | |
| | | | Rubella virus (strain BRDII) | | |
| | | | Rubella virus (strain Cendehill) | | |
| | | | Rubella virus (strain M33) | | |
| | | | Rubella virus (strain RN-UK86) | | |
| | | | Rubella virus (strain THERIEN) | | |
| | | | Rubella virus (strain TO-336 vaccine) | | |
| | | | Rubella virus (strain TO-336) | | |
| | | | Rubella virus (vaccine strain RA27/3) | | |
| Bunya- | Hanta-virus | Amur virus | | Gn(G2)/Gc(G1) | |
| viridae | (continued on | Bayou virus | | | |
| | next page) | Black Creek Canal | | | |
| | | virus | | | |
| | | Cano Delgadito | | | |
| | | virus | | | |
| | | Calabazo virus | | | |
| | | Catacamas virus | | | |
| | | Choclo virus | | | |
| | | Dobrava-Belgrade | | | |
| | | virus | | | |
| | | El Moro Canyon | | | |
| | | virus | | | |
| | | Hantaan virus | | | |
| | | Isla Vista virus | | | |
| | | Khabarovsk virus | | | |
| | | Laguna Negra | | | |
| | | virus | | | |
| | | Limestone Canyon | | | |
| | | virus | | | |
| | | Monongahela virus | | | |
| | | Muleshoe virus | | | |
| | | Muju virus | | | |
| | | New York virus | | | |
| | | Oran virus | | | |
| | | Playa de Oro | | | |
| | | virus | | | |
| | | Prospect Hill | | | |
| | | virus | | | |
| | | Puumala virus | | | |
| | | Rio Mamore virus | | | |
| | | Rio Segundo virus | | | |
| | | Saaremaa virus | | | |
| | | Seoul virus | | | |
| | | Sin Nombre virus | | | |
| | | Soochong virus | | | |
| | | Thailand virus | | | |
| | | Thottapalayam | | | |
| | | virus | | | |
| | | Topografov virus | | | |
| | | Tula virus | | | |
| | Ortho-bunya- | Anopheles A virus | | | |
| | virus | Anopheles B virus | | | |
| | | Bakau virus | | | |
| | | Batama virus | | | |
| | | Bwamba virus | | | |
| | | Caraparu virus | | | |
| | | Kaeng Khoi virus | | | |
| | | Kairi virus | | | |
| | | Madrid virus | | | |
| | | Main Drain virus | | | |
| | | Marituba virus | | | |
| | | Nyando virus | | | |
| | | Oriboca virus | | | |
| | | Oropouche virus | | | |
| | | Sathuperi virus | | | |
| | | Shamonda virus | | | |
| | | Shuni virus | | | |
| | | Simbu virus | | | |
| | | Tacaiuma virus | | | |
| | | Tete virus | | | |
| | | Turlock virus | | | |
| | | unclassified | | | |
| | | Orthobunyavirus | | | |
| | | Akabane virus | Sabo virus | | |
| | | | Tinaroo virus | | |
| | | | Yaba-7 virus | | |
| | | Bunyamwera virus | Batai virus | | |
| | | | Birao virus | | |
| | | | Bozo virus | | |
| | | | Cache Valley virus | | |
| | | | Fort Sherman virus | | |
| | | | Germiston virus | | |
| | | | Guaroa virus | | |
| | | | Iaco virus | | |
| | | | Ilesha virus | | |
| | | | Lokern virus | | |
| | | | Maguari virus | | |
| | | | Mboke virus | | |
| | | | Ngari virus | | |
| | | | Northway virus | | |
| | | | Playas virus | | |
| | | | Potosi virus | | |
| | | | Shokwe virus | | |
| | | | Tensaw virus | | |
| | | | Tlacotalpan virus | | |
| | | | Xingu virus | | |
| | | California | California encephalitis serogroup virus | | |
| | | Encephalitis | LEIV | | |
| | | virus | California encephalitis virus - BFS-283 | | |
| | | | Chatanga virus | | |
| | | | Inkoo virus | | |
| | | | Jamestown Canyon virus | | |
| | | | Jamestown Canyon-like virus | | |
| | | | Jerry Slough virus | | |
| | | | Keystone virus | | |
| | | | La Crosse virus | | |
| | | | Lumbo virus | | |
| | | | Melao virus | | |
| | | | Morro Bay virus | | |
| | | | San Angelo virus | | |
| | | | Serra do Navio virus | | |
| | | | Snowshoe hare virus | | |
| | | | South River virus | | |
| | | | Tahyna virus | | |
| | | | Trivittatus virus | | |
| | | Caraparu virus | Apeu virus | | |
| | | | Bruconha virus | | |
| | | | Ossa virus | | |
| | | | Vinces virus | | |
| | | Manzanilla virus | Buttonwillow virus | | |
| | | | Ingwavuma virus | | |
| | | | Mermet virus | | |
| | | Marituba virus | Gumbo Limbo virus | | |
| | | | Murutucu virus | | |
| | | | Nepuyo virus | | |
| | | | Restan virus | | |
| | | Wyeomyia virus | Anhembi virus | | |
| | | | BeAr328208 virus | | |
| | | | Macaua virus | | |
| | | | Sororoca virus | | |
| | | | Taiassui virus | | |
| | Phlebovirus | Bujaru virus | | | |
| | | Candiruvirus | | | |
| | | Chilibre virus | | | |
| | | Frijoles virus | | | |
| | | Punta | | | |
| | | Tor_\|Salehabad | | | |
| | | virus | | | |
| | | Sandflyfever | | | |
| | | Naples virus | | | |
| | | Uukuniemi viruso | | | |
| | | virus | | | |
| | | Rift Valley | | | |
| | | fever virus | | | |
| | | unclassified | Anhanga virus | | |
| | | Phlebovirus | Arumowot virus | | |
| | | (continued on | Chagres virus | | |
| | | next page) | Corfou virus | | |
| | | | Gabek Forest virus | | |
| | | | Itaporanga virus | | |
| | | | Phlebovirus Adria/ALB1/2005 | | |
| | | | Phlebovirus Adria/ALB5/2005 | | |
| | | | Phlebovirus AH12 | | |
| | | | Phlebovirus AH12/China/2010 | | |
| | | | Phlebovirus AH15/China/2010 | | |
| | | | Phlebovirus B105-05 | | |
| | | | Phlebovirus B151-04 | | |
| | | | Phlebovirus B43-02 | | |
| | | | Phlebovirus B68-03 | | |
| | | | Phlebovirus B79-02 | | |
| | | | Phlebovirus Chios-A | | |
| | | | Phlebovirus Cyprus | | |
| | | | Phlebovirus HB29/China/2010 | | |
| | | | Phlebovirus HN13/China/2010 | | |
| | | | Phlebovirus HN6/China/2010 | | |
| | | | Phlebovirus Hu/Xinyang1/China/2010 | | |
| | | | Phlebovirus Hu/Xinyang2/China/2010 | | |
| | | | Phlebovirus IB13-04 | | |
| | | | Phlebovirus JS2007-01 | | |
| | | | Phlebovirus JS24 | | |
| | | | Phlebovirus JS26 | | |
| | | | Phlebovirus JS3/China/2010 | | |
| | | | Phlebovirus JS4/China/2010 | | |
| | | | Phlebovirus JS6 | | |
| | | | Phlebovirus JSD1 | | |
| | | | Phlebovirus LN2/China/2010 | | |
| | | | Phlebovirus LN3/China/2010 | | |
| | | | Phlebovirus sandflies/Gr29/Spain/2004 | | |
| | | | Phlebovirus sandflies/Gr36/Spain/2004 | | |
| | | | Phlebovirus sandflies/Gr44/Spain/2004 | | |
| | | | Phlebovirus sandflies/Gr49/Spain/2004 | | |
| | | | Phlebovirus sandflies/Gr52/Spain/2004 | | |
| | | | Phlebovirus sandflies/Gr65/Spain/2004 | | |
| | | | Phlebovirus sandflies/Gr98/Spain/2004 | | |
| | | | Phlebovirus SD24/China/2010 | | |
| | | | Phlebovirus SD4/China/2010 | | |
| | | | Phlebovirus tick/XCQ-2011 | | |
| | | | Phlebovirus XLL/China/2009 | | |
| | | | Rio Grande virus | | |
| | | | Salobo virus | | |
| | | | Sandfly fever sicilian virus | | |
| | | | Sandfly Sicilian Turkey virus | | |
| | | | Utique virus | | |
| | | | Phlebovirus sp. | | |
| | | | Phlebovirus sp. Be An 24262 | | |
| | | | Phlebovirus sp. Be An 356637 | | |
| | | | Phlebovirus sp. Be An 416992 | | |
| | | | Phlebovirus sp. Be An 578142 | | |
| | | | Phlebovirus sp. Be Ar 371637 | | |
| | | | Phlebovirus sp. Co Ar 170255 | | |
| | | | Phlebovirus sp. Co Ar 171616 | | |
| | | | Phlebovirus sp. GML 902878 | | |
| | | | Phlebovirus sp. Pa Ar 2381 | | |
| | | | Phlebovirus sp. PAN 479603 | | |
| | | | Phlebovirus sp. PAN 483391 | | |
| | | | Phlebovirus sp. VP-161A | | |
| | | | Phlebovirus sp. VP-334K | | |
| | | | Phlebovirus sp. VP-366G | | |
| Orthomyxo- | Influenzavirus A | Influenza | INFA H1 | HA | Group 2 Type I |
| viridae | | A virus | | (HA1/HA2) | fusion mechanism |
| | | | INFA H2 | | |
| | | | INFA H3 | | |
| | | | INFA H4 | | |
| | | | INFA H5 | | |
| | | | INFA H6 | | |
| | | | INFA H7 | | |
| | | | INFA H8 | | |
| | | | INFA H9 | | |
| | | | INFA H10 | | |
| | | | INFA H11 | | |
| | | | INFA H12 | | |
| | | | INFA H13 | | |
| | | | INFA H14 | | |
| | | | INFA H15 | | |
| | | | INFA H16 | | |
| | Influenzavirus B | Influenza B virus | All strains | | |
| | Influenzavirus C | Influenza C virus | | | |
| Paramyxo- | Paramyxovirinae | Avulavirus | Avian paramyxovirus 2 Yucaipa virus | F0 | |
| viridae | | | Avian paramyxovirus 3 | (F2/F1) | |
| | | | Avian paramyxovirus 3b | | |
| | | | Avian paramyxovirus 4 | | |
| | | | Avian paramyxovirus 5 | | |
| | | | Avian paramyxovirus 6 | | |
| | | | Avian paramyxovirus 7 | | |
| | | | Avian paramyxovirus 8 | | |
| | | | Avian paramyxovirus 9 | | |
| | | | Newcastle disease virus | | |
| | | | Pigeon paramyxovirus 1 | | |
| | | | unclassified Avulavirus | | |
| | | | Avian paramyxovirus 10_Avian | | |
| | | | paramyxovirus duck/Miyagi/885/05 | | |
| | | | Avian paramyxovirus penguin/Falkland | | |
| | | | Islands/324/2007 | | |
| | | | Goosramyxovirus HZ | | |
| | | | Goose paramyxovirus JS/1/97/Go | | |
| | | | Goose paramyxovirus SF02 | | |
| | | Henipavirus | Hendra virus Hendra virus | | |
| | | | horse/Australia/Hendra/1994 | | |
| | | | Nipah virus | | |
| | | | unclassified Henipavirus | | |
| | | | Bat paramyxovirus | | |
| | | | Eid.hel/GH45/2008 | | |
| | | Morbillivirus | Canine distemper virus | | |
| | | | Cetacean morbillivirus_Dolphin | | |
| | | | morbillivirus_Pilot whale morbillivirus | | |
| | | | Porpoise morbillivirus | | |
| | | | Measles virus | | |
| | | | Peste-des-petits-ruminants virus | | |
| | | | Phocine distemper virus | | |
| | | | Phocine distemper virus 1 | | |
| | | | Phocine distemper virus-2 | | |
| | | | Rinderpest virus | | |
| | | Respirovirus | Bovine parainfluenza virus 3 | | |
| | | | Porcine paramyxovirus strain Frost | | |
| | | | Porcine paramyxovirus strain Texas | | |
| | | | Human parainfluenza virus 1 | | |
| | | | Human parainfluenza virus 3 | | |
| | | | Simian Agent 10 | | |
| | | | Sendai virus | | |
| | | | unclassified Respirovirus | | |
| | | | Atlantic salmon respirovirus | | |
| | | | Guinea pig parainfluenza virus TS-9 | | |
| | | | Pacific salmon paramyxovirus | | |
| | | | Trask River 1983 Swine parainfluenza | | |
| | | | virus 3 | | |
| | | | Tursiops truncatus parainfluenza virus 1 | | |
| | | Rubulavirus | Human parainfluenza virus 2 | | |
| | | | Human parainfluenza virus 2 (strain | | |
| | | | Greer) | | |
| | | | Human parainfluenza virus 2 (strain | | |
| | | | Toshiba) | | |
| | | | Human parainfluenza virus 4 | | |
| | | | Human parainfluenza virus 4a | | |
| | | | Human parainfluenza virus 4b | | |
| | | | Mapuera virus | | |
| | | | Mumps virus | | |
| | | | Parainfluenza virus 5 | | |
| | | | Porcine rubulavirus | | |
| | | | Simian virus 41 | | |
| | | | unclassified Rubulavirus | | |
| | | | Porcine parainfluenza virus | | |
| | | | Tuhoko virus 1 | | |
| | | | Tuhoko virus 2 | | |
| | | | Tuhoko virus 3 | | |
| | | unclassified | Atlantic salmon paramyxovirus | | |
| | | Paramyxovirinae | Beilong virus | | |
| | | | Canine parainfluenza virus | | |
| | | | Chimeric human parainfluenza virus | | |
| | | | rPIV3-2 | | |
| | | | Fer-de-lance virus | | |
| | | | J-virus | | |
| | | | Menangle virus | | |
| | | | Mossman virus | | |
| | | | Murayama virus | | |
| | | | Ovine parainfluenza virus 3 | | |
| | | | Pacific salmon paramyxovirus | | |
| | | | Paramyxovirus GonoGER85 | | |
| | | | Recombinant PIV3/PIV1 virus | | |
| | | | Reptilian paramyxovirus | | |
| | | | Salem virus | | |
| | | | Salmo salar paramyxovirus | | |
| | | | Snake ATCC-VR-1408 paramyxovirus | | |
| | | | Snake ATCC-VR-1409 paramyxovirus | | |
| | | | Tioman virus | | |
| | | | Tupaia paramyxovirus | | |
| | Pneumovirus | Human respiratory | Human respiratory syncytial virus A | | Group 3 |
| | | syncytial virus | Human respiratory syncytial virus | | Type I fusion mechanism |
| | | | (strain RSB1734) | | |
| | | | Human respiratory syncytial virus | | |
| | | | (strain RSB5857) | | |
| | | | Human respiratory syncytial virus | | |
| | | | (strain RSB6190) | | |
| | | | Human respiratory syncytial virus | | |
| | | | (strain RSB6256) | | |
| | | | Human respiratory syncytial virus | | |
| | | | (strain RSB642) | | |
| | | | Human respiratory syncytial virus | | |
| | | | (strain RSB6614) | | |
| | | | Human respiratory syncytial virus A | | |
| | | | strain Long LinkOut | | |
| | | | Human respiratory syncytial virus A2 | | |
| | | | Human respiratory syncytial virus B | | |
| | | | Human respiratory syncytial | | |
| | | | virus (subgroup B/strain 18537) | | |
| | | | Human respiratory syncytial virus | | |
| | | | (subgroup B strain 8/60) | | |
| | | | Human Respiratory syncytial virus 9320 | | |
| | | | Human respiratory syncytial virus B1 | | |
| | | | Human respiratory syncytial virus S2 | | |
| | | | Human respiratory syncytial virus strain | | |
| | | | RSS-2 | | |
| | | | unclassified Human respiratory syncytial | | |
| | | | virus | | |
| | | Bovine | All strains | | Group 3 |
| | | respiratory | | | Type I |
| | | syncytial virus | | | fusion |
| | Metapneum | Avian metapneumo- | All strains | | |
| | | virus | | | |
| | | Human metapneumo- | All strains | | |
| | | virus | | | |
| Corona- | Coronavirinae | Alphacorona- | Alphacoronavirus 1 | S | Group 3 Type I |
| viridae | | virus | Coronavirus group 1b | (S1/S2) | fusion mechanism |
| | | | Human coronavirus 229E | | |
| | | | Human coronavirus NL63 | | |
| | | | Miniopterus bat coronavirus 1 | | |
| | | | Miniopterus bat coronavirus HKU8 | | |
| | | | Porcine epidemic diarrhea virus | | |
| | | | Rhinolophus bat coronavirus HKU2 | | |
| | | | Scotophilus bat coronavirus 512 | | |
| | | | unclassified Alphacoronavirus | | |
| | | Betacorona- | Betacoronavirus 1 | | |
| | | virus | Coronavirus group 2b | | |
| | | | Coronavirus group 2c | | |
| | | | Human coronavirus HKU1 | | |
| | | | Murine coronavirus | | |
| | | | Pipistrellus bat coronavirus HKU5 | | |
| | | | Rousettus bat coronavirus HKU9 | | |
| | | | Severe acute respiratory syndrome- | | |
| | | | related coronavirus recombinant SARSr- | | |
| | | | CoV | | |
| | | | SARS coronavirus | | |
| | | | Tylonycteris bat coronavirus HKU4 | | |
| | | | unclassified Betacoronavirus | | |
| | | Gammacorona- | Avian coronavirus | | |
| | | virus | Beluga Whale coronavirus SW1 | | |
| | | unclassified | Alpaca coronavirus CA08-1/2008 | | |
| | | coronaviruses | Bat coronavirus | | |
| | | | Bird droppings coronavirus | | |
| | | | Bovine respiratory coronavirus | | |
| | | | Chicken enteric coronavirus | | |
| | | | Coronavirus Anas | | |
| | | | Coronavirus oystercatcher/p17/2006/GBR | | |
| | | | Coronavirus red knot/p60/2006/GBR | | |
| | | | Ferret enteric coronavirus 1202 | | |
| | | | Ferret systemic coronavirus MSU-S | | |
| | | | Ferret systemic coronavirus WADL | | |
| | | | Guangxi coronaviridae | | |
| | | | Human coronavirus NO | | |
| | | | Human enteric coronavirus strain 4408 | | |
| | | | Kenya bat coronavirus | | |
| | | | Mink coronavirus strain WD1133 | | |
| | | | Parrot coronavirus AV71/99 | | |
| | | | Quail coronavirus Italy/Elvia/2005 | | |
| | | | Tai Forest coronavirus | | |
| | | | unidentified coronavirus | | |
| | | | unidentified human coronavirus | | |
| Arena- | Arena-virus | LCMV-Lassa virus | Ippy virus | GpC | Group 3 Type I |
| viridae | | (Old World) | Lassa virus | (Gp1/Gp2) | fusion mechanism |
| | | complex | Lujo virus | | |
| | | | Lymphocytic choriomeningitis virus | | |
| | | | Mobala virus | | |
| | | | Mopeia virus | | |
| | | Tacaribe virus | Amapari virus | | |
| | | (New World) | Chapare virus | | |
| | | complex | Flexal virus | | |
| | | | Guanarito virus | | |
| | | | Junin virus | | |
| | | | Latino virus | | |
| | | | Machupo virus | | |
| | | | Oliveros virus | | |
| | | | Parana virus | | |
| | | | Pichinde virus | | |
| | | | Pirital virus | | |
| | | | Sabia virus | | |
| | | | Tacaribe virus | | |
| | | | Tamiami virus | | |
| | | | Whitewater Arroyo virus | | |
| Hepadna- | Genus | Hepatitis B virus | HBV genotype A | L and M and S | Group 3 Fusion |
| viridae | | Orthohepadnavirus | HBV genotype B | Where S mediates fusion | mechanism - Neither |
| | | | HBV genotype C | | type I nor type II |
| | | | HBV genotype D | | |
| | | | HBV genotype E | | |
| | | | HBV genotype F | | |
| | | | HBV genotype G | | |
| | | | HBV genotype H | | |
| | | | Hepatitis B virus alpha1 | | |
| | | | Hepatitis B virus LSH/chimpanzee | | |
| | | | Hepatitis B virus strain cpz | | |
| | | | Hepatitis B virus subtype adr | | |
| | | | Hepatitis B virus subtype adw | | |
| | | | Hepatitis B virus subtype adyw | | |
| | | | Hepatitis B virus subtype ayw | | |
| Rhabdo- | Dimarhabdovirus | Ephemerovirus | Bovine ephemeral fever virus | Glycoprotein G | Group 3 |
| viridae | | | | | Neither type I nor |
| | | | | | Type II fusion |
| | | | | | mechanism |
| | | Vesiculovirus | Carajas virus | | |
| | | | Chandipura virus | | |
| | | | Cocal virus | | |
| | | | Isfahan virus | | |
| | | | Maraba virus | | |
| | | | Piry virus | | |
| | | | recombinant Vesiculovirus | | |
| | | | Spring viraemia of carp virus | | |
| | | | Vesicular stomatitis Alagoas virus | | |
| | | | Vesicular stomatitis Indiana virus | | |
| | | | Vesicular stomatitis New Jersey virus | | |
| | Lyssavirus | Aravan virus | | | |
| | | Australian bat | | | |
| | | lyssavirus | | | |
| | | Duvenhage virus | | | |
| | | European bat | | | |
| | | lyssavirus 1 | | | |
| | | European bat | | | |
| | | lyssavirus 2 | | | |
| | | Irkut virus | | | |
| | | Khujand virus | | | |
| | | Lagos bat virus | | | |
| | | Mokola virus | | | |
| | | West Caucasian | | | |
| | | bat virus | | | |
| | | Rabies virus | Rabies virus AB21 | | |
| | | | Rabies virus AB22 | | |
| | | | Rabies virus AVO1 | | |
| | | | Rabies virus BNG4 | | |
| | | | Rabies virus BNG5 | | |
| | | | Rabies virus China/DRV | | |
| | | | Rabies virus China/MRV | | |
| | | | Rabies virus CVS-11 | | |
| | | | Rabies virus ERA | | |
| | | | Rabies virus Eth2003 | | |
| | | | Rabies virus HEP-FLURY | | |
| | | | Rabies virus India | | |
| | | | Rabies virus Nishigahara RCEH | | |
| | | | Rabies virus Ontario fox | | |
| | | | Rabies virus Ontario skunk | | |
| | | | Rabies virus PM | | |
| | | | Rabies virus red fox/08RS- | | |
| | | | 1981/Udine/2008 | | |
| | | | Rabies virus SAD B19 | | |
| | | | Rabies virus silver-haired bat- | | |
| | | | associated SHBRV | | |
| | | | Rabies virus strain Pasteur vaccin | | |
| | | | Rabies virus strain Street | | |
| | | | Rabies virus vnukovo-32 | | |
| | | | Thailand genotype 1 dog lyssavirus | | |
| | | unclassified | Bokeloh bat lyssavirus | | |
| | | Lyssavirus | European bat lyssavirus | | |
| | | | Lyssavirus Ozernoe | | |
| | | | Shimoni bat virus | | |
| | Novirhabdovirus | Hirame | | | |
| | | rhabdovirus | | | |
| | | Infectious | | | |
| | | hematopoietic | | | |
| | | necrosis virus | | | |
| | | Snakehead | | | |
| | | rhabdovirus | | | |
| | | Viral hemorrhagic | | | |
| | | septicemia virus | | | |
| | unassigned | Bangoran virus | | | |
| | Rhabdoviridae | Bimbo virus | | | |
| | | Bivens Arm virus | | | |
| | | Flanders virus | | | |
| | | Garba virus | | | |
| | | Klamath virus | | | |
| | | Malpais Spring | | | |
| | | virus | | | |
| | | Nasoule virus | | | |
| | | Ngaingan virus | | | |
| | | Ouango virus | | | |
| | | Sigma virus | | | |
| | | Tupaia virus | | | |
| | | Wongabel virus | | | |

According to an embodiment, the invention concerns a method for identifying an immunosuppressive domain in the fusion protein of an enveloped RNA virus having a lipid membrane, said method comprising:
a. Identifying at least one well conserved domain among the group consisting of the membrane associated domains of the fusion protein and the surface associated domains of the fusion protein;
b. Providing at least one peptide with the sequence of said identified at least one well conserved domain;
c. Optionally dimerizing or multimerizing said at least one peptide; and
d. Confirming the immunosuppressive activity of said at least one optionally dimerized or multimerized peptide by testing said at least one optionally dimerized or multimerized peptide for immunosuppressive activity.

The at least one well conserved domain may be identified among domains, which are membrane associated and domains, which are surface associated. Naturally, a domain which is both membrane and surface associated may be a well conserved domain.

The fusion protein may be identified by searching NCBI taxonomy (www.ncbi.nlm.nih.gov/Taxonomy/), and selecting proteins of the Family, Subfamily, Genus or Species to be investigated, and subsequently search these for fusion or the specific name of the fusion protein, e.g. as listed in Table 1.

The dimerized peptide could be synthetic, the multimerized peptide could be displayed as dimerized or trimerized fusion proteins either displayed alone or on membranes such as a viral particle.

One way of testing the immunosuppressive activity of the at least one dimerized or multimerized peptide is to test the immunosuppressive activity of the fusion protein in the absence and presence of the at least one dimerized or multimerized peptide, and comparing the results.

According to other embodiments, the invention concerns the method, wherein the identification of said at least one well conserved domain is done among the group consisting of the surface associated domains of the fusion protein in one or more of the different conformations of the fusion protein undergoing fusion.

According to an embodiment, the invention concerns a method, wherein the enveloped RNA virus is not selected among Retroviruses, Lentiviruses or Filoviruses. In particular, according to an embodiment, the invention concerns a method, wherein said at least one well conserved immunosuppressive domain is not located in the linker between the two heptad repeat structures just N-terminal of the transmembrane domain in the fusion protein of either Retrovirus, Lentivirus or Filovirus. More particularly, according to an embodiment, the invention concerns a method, wherein said at least one well conserved domain does not include some of the 22 amino acids located N-terminal to the first of two well conserved cysteine residues that are found in these structures in the fusion protein of either Retrovirus, Lentivirus or Filovirus. These cysteine residues are between 4 and 6 amino acid residues from one another and in many cases are believed to form disulfide bridges that stabilize the fusion proteins.

According to other embodiments, the invention concerns the method, wherein said at least one well conserved domain is selected among the group consisting of Putative ISUs and Identified ISUs of Table 1 and Seq. Id. 1-200.

According to an embodiment, the invention concerns an immunosuppressive domain identified according to the invention.

According to an embodiment, the invention concerns an immunosuppressive domain selected among the sequences of Table 1 and Seq. Id. 1-200.

According to an embodiment, the invention concerns a method for decreasing or completely abrogating the immunosuppressive properties of an immunosuppressive domain of the fusion protein of an enveloped RNA virus having a lipid membrane, said method comprising the steps of:
e. Mutating an immunosuppressive domain to provide at least one mutated peptide;
f. Optionally dimerizing or multimerizing said at least one mutated peptide;
g. Selecting one of said optionally dimerized or multimerized mutated peptides showing reduced immunosuppressive properties;
h. Mutating the fusion protein of the enveloped RNA virus to contain said selected mutated peptide having reduced immunosuppressive properties;
i. Confirming expression by testing the viral envelope protein encompassing said mutated fusion protein for capability of being expressed by at least one of cellular or viral surfaces.

The envelope protein may be identified by searching NCBI taxonomy (www.ncbi.nlm.nih.gov/Taxonomy/) and selecting proteins of the Family, Subfamily, Genus or Species to be investigated and subsequently searching these for envelope or the specific name for the envelope protein or the attachment and fusion protein, e.g. as listed in Table 1.

According to other embodiments, the invention concerns the method, wherein:
e. Said immunosuppressive domain is mutated to provide a plurality of mutated peptides;
f. Said plurality of mutated peptides are optionally dimerized or multimerized;
g. One of said optionally dimerized or multimerized mutated peptides showing reduced immunosuppressive properties is selected;
h. The fusion protein of the enveloped RNA virus is mutated to contain said selected optionally dimerized or multimerized peptide having reduced immunosuppressive properties;
i. Expression is confirmed by testing the viral envelope protein encompassing said mutated fusion protein for capability of being expressed by at least one of cellular or viral surfaces.

According to other embodiments, the invention concerns the method, wherein:
g. One of said optionally dimerized or multimerized mutated peptide(s) is selected, which has reduced immunosuppressive properties as shown by at least 25% reduction as compared to a dimerized Wildtype peptide.

According to other embodiments, the invention concerns the method, wherein:
e. Said mutated immunosuppressive domain is mutated to provide a knock-out mutant of Table 1 or selected among the sequences of Seq. Id. 201-203.

According to an embodiment, a proven knock-out (i.e. a mutation of the immunosuppressive domain abrogating the immunosuppressive properties of the peptide) from one family, genus, group and/or strain, may be used for another family, genus, group and/or strain.

According to an embodiment, the invention concerns a mutated peptide providing reduced immunosuppressive properties, said mutated peptide having a sequence according to Table 1 or any of Seq. Id.—202 to 203 or obtainable as said selected mutated peptide of a method according to the invention.

Preliminary experiments indicate the immunosuppressive domains may have a size of 4-30 amino acids.

According to an embodiment, the invention concerns a method for generating an enhanced immune response, comprising a method according to the invention, and further comprising the step of:
j. Using said viral envelope protein encompassing said mutated fusion protein with reduced immunosuppressive properties as an antigen for generation of an enhanced immune response.

According to an embodiment, the invention concerns a method for making an envelope protein having diminished immunosuppressive activity, comprising:
Mutating or modifying an immunosuppressive domain, identifiable according to the invention, of an enveloped RNA virus with a lipid membrane surrounding the core, to include a peptide obtainable according to the invention.

The diminished immunosuppressive activity is suitably measured by comparing to the immunosuppressive activity from an envelope of a wildtype peptide. It is preferably demonstrated by an increased proliferation of at least 25% in a cell proliferation assay of homodimers of said mutated peptide as compared to the homodimers of said non-mutated wildtype peptide at the same concentration. More preferably the cell assay is either the CTLL-2 or the PBMC assay.

According to an embodiment, the invention concerns the method, for making a envelope protein encompassing a mutated fusion protein from a enveloped RNA virus for medical use, such as therapeutic or prophylactic purpose, preferably for use as a vaccine.

According to an embodiment, the invention concerns the method, for making an enveloped protein encompassing a mutated fusion protein from an envelope RNA virus for vaccination purposes or for the generation of neutralizing antibodies.

According to an embodiment, the invention concerns the method, wherein the enveloped RNA virus has a fusion protein with a type II fusion mechanism.

According to an embodiment, the invention concerns the method, wherein the enveloped RNA virus, preferably excluding lentivius, retrovirus and filovirus, has a fusion protein with a type I fusion mechanism and where the immunosuppressive domains co-localizes with the fusion peptide in the fusion protein, preferably as demonstrated by the identification of a common immunosuppressive domain in the fusion peptide of all H1 to H16 of Influenza A and influenza B.

According to an embodiment, the invention concerns the method, wherein the enveloped RNA virus, preferably excluding lentivius, retrovirus and filovirus, has a fusion protein with a type I fusion mechanism excluding viruses with a type I fusion mechanism where the ISU co-localizes with the fusion peptide or the fusion protein has a structure that is neither a type I nor a type II fusion structure.

According to an embodiment, the invention concerns an envelope protein obtainable according to the invention.

The immunosuppressive domain has so far been identified by the inventors at two positions in two different groups of viruses A: Co-localizing with the fusion peptide exemplified by the identification of an common immunosuppressive domain in the fusion peptide of all Flavirus (Dengue virus, west Nile virus etc) and Influenza A and B viruses and B: in the hydrophobic alpha helix N-terminal of the transmembrane domain in the fusion protein exemplified by the finding of an immunosuppressive domain in said helixes of Flaviridae like e.g. Hepatitis C virus, Dengue, WestNile, Yellow fever.

The inventors have realized that the potential immunosuppressive domains are located at various positions in the fusion protein identifiable by
1): The peptide is preferably located in the fusion protein of enveloped RNA viruses;
2): The peptide is preferably capable of interacting with membranes;
3): Preferably a high degree of homology in the primary structure (sequence) of the peptide of said domain exists either within the viral species itself, in the family of viruses or in a group of viruses. This requirement is due to the immunosuppressive domain being under a dual selection pressures, one as an immunosuppressive entity ensuring protection of the viral particle from the host immune system, another as a peptide interacting with membranes; and/or
4): The position at the surface of the fusion protein at a given conformation is preferably a feature of immunosuppressive domains. This can be revealed either by position in a 3D structure or by antibody staining of cells expressing the fusion protein or on viral surfaces displaying the fusion protein.

According to an embodiment, the invention concerns a mutated envelope protein according to the invention.

According to an embodiment, the invention concerns a viral fusion protein from an enveloped RNA virus with reduced immunosuppressive properties, said fusion protein encompassing a mutated peptide, said mutated peptide displaying reduced immunosuppression, and said mutated peptide replacing an un-mutated wildtype peptide having a sequence of an ISU of Table 1 or is selected among Seq. Id. 1-200.

According to an embodiment, the invention concerns the fusion protein, where the reduced immunosuppression is identified by comparing to the un-mutated wildtype peptide when said peptide is dimerized.

According to an embodiment, the invention concerns the fusion protein, wherein said immunosuppressive activity being determined by at least 25% reduction, more preferred at least 40% reduction, in proliferation rate in a cell proliferation assay using a homodimer of said un-mutated peptide compared to the monomeric version of said peptide at the same concentration.

According to an embodiment, the invention concerns the fusion protein, wherein said cell proliferation assay is selected among the group consisting of the CTLL-2 and the PBMC assay.

According to an embodiment, the invention concerns the fusion protein, wherein said fusion protein has a type I or type II fusion mechanism.

According to an embodiment, the invention concerns the fusion protein, wherein said fusion protein has neither a type I nor type II fusion mechanism.

According to an embodiment, the invention concerns the fusion protein, wherein said mutated peptide is located either in the fusion peptide or in a, preferably amphipatic, helix upstream of the C-terminal transmembrane domain of said fusion protein.

The fusion peptide is a small membrane penetrating peptide located in the fusion protein of enveloped viruses.

According to another embodiment, the invention concerns the viral fusion protein, wherein said mutated peptide is derived from the fusion peptide from a flavivirus or Influenzavirus or from the amphipatic helix of the Flaviridae, such as the group consisting of Hepatitis C virus fusion protein, Dengue virus fusion protein, and WestNile virus fusion protein.

According to an embodiment, the invention concerns an envelope protein, said mutated fusion protein being displayed on the surface of cells wherein said mutated fusion protein is expressed.

According to an embodiment, the invention concerns the envelope protein, said mutated fusion protein being displayed on the surface of viral or viral like particles.

According to an embodiment, the invention concerns the envelope protein, having retained some fusiogenic activity.

According to an embodiment, the invention concerns the envelope protein, wherein the fusiogenic activity is measured by a technique for measuring cell-cell fusion, preferably selected among the group consisting of counting syncytia by light microscopy, resonance energy transfer based assays, and indirect reporter gene using techniques or by measuring infectious titers; alternatively, or in addition, the presence of fusiogenic activity may be indicated by the presence of at least one cell expressing the modified envelope and one cell expressing the receptor and/or coreceptors being fused together.

According to an embodiment, the invention concerns an enveloped RNA virus, different from a virus selected among the group consisting of Retrovirus, Lentivirus and Filovirus, wherein an immunosuppressive domain has been modified or mutated to decrease or completely abrogate the immunosuppressive properties of an immunosuppressive domain of the fusion protein.

According to an embodiment, the invention concerns a virus selected among the vira of Table 1, wherein an immunosuppressive domain has been modified or mutated to decrease or completely abrogate the immunosuppressive properties of an immunosuppressive domain of the fusion protein.

According to an embodiment, the invention concerns an antigen obtainable by selecting a part of a mutated envelope protein according to any of the preceding claims, said part comprising the mutated domain of said envelope protein.

According to an embodiment, the invention concerns an antigen comprising an mutated immunosuppressive domain selected among the sequences of Table 1 and Seq. Id. 201 to 202.

According to an embodiment, the invention concerns an antigen of the invention furthermore harboring 1 or 2 or 3 or 4 or 5 or 6 or 7 or 8 or 9 or 10 or 11 point mutation(s) in any of the sequences of Table 1 or of Seq. Id. 1-200.

According to an embodiment, the invention concerns an antigen, which mediates fusion of virus to host cells.

According to an embodiment, the invention concerns an antigen, which is recombinant or obtained by recombinant technology.

According to an embodiment, the invention concerns a nucleic acid sequence, preferably recombinant, encoding a mutated envelope protein, an envelope polypeptide or an antigen according to any of the preceding claims.

According to an embodiment, the invention concerns an isolated eukaryotic expression vector comprising a nucleic acid sequence according to the invention.

According to another embodiment, the invention concerns the vector, which is a virus vector, preferably a virus selected among the group consisting of vaccinia virus, measles virus, retroviridae, lentivirus, baculovirus and adeno virus.

According to an embodiment, the invention concerns a method for producing an antibody, said method comprising the steps of:
Administering an entity selected among a mutated envelope, an envelope polypeptide, an antigen, a nucleic acid sequence or a vector according to any of the preceding claims to a host, such as an animal; and
Obtaining the antibody from said host.

According to an embodiment, the invention concerns an antibody obtainable according to a method of the invention.

According to another embodiment, the invention concerns an antibody, which is specific for an entity selected among a mutated peptide, an envelope protein, a mutated envelope protein, an antigen, a nucleic acid sequence or a vector according to any of the preceding claims.

According to an embodiment, the invention concerns neutralizing antibodies obtained or identified by the use of at least one envelope protein according to any of the preceding claims.

According to an embodiment, the invention concerns a method for manufacturing neutralizing antibodies comprising the use of at least one protein according to any of the preceding claims.

According to an embodiment, the invention concerns a method for manufacturing humanized neutralizing antibodies, comprising the use of at least one sequence selected among the sequences of Table 1 and sequences 201 to 203

According to an embodiment, the invention concerns a vaccine comprising a virus according to the invention.

According to an embodiment, the invention concerns a vaccine comprising an envelope protein from a virus according to the invention.

According to an embodiment, the invention concerns a vaccine composition comprising an envelope protein according to any of the preceding claims.

According to an embodiment, the invention concerns a vaccine composition comprising a virus like particle (VLP).

According to an embodiment, the invention concerns the vaccine composition, wherein the virus like particle is produced ex vivo in a cell culture.

According to an embodiment, the invention concerns the vaccine composition, wherein the virus like particle is partly or completely assembled ex vivo.

According to an embodiment, the invention concerns the vaccine composition, wherein the virus like particle is generated in vivo in the patient by infection, transfection and/or electroporation by expression vectors.

According to an embodiment, the invention concerns the vaccine composition, comprising a vector derived from a measles or vaccinia virus.

According to an embodiment, the invention concerns the vaccine composition, comprising an expression vector for DNA vaccination.

According to an embodiment, the invention concerns the vaccine composition, comprising a purified envelope protein.

According to an embodiment, the invention concerns the vaccine composition, comprising a multimerized purified envelope protein.

According to an embodiment, the invention concerns the vaccine composition, comprising a dimerized purified envelope protein.

According to an embodiment, the invention concerns the vaccine composition, comprising a trimerized purified envelope protein.

According to an embodiment, the invention concerns a vaccine composition comprising an entity selected among the group consisting of a mutated envelope protein, an envelope polypeptide, an antigen, a nucleic acid sequence, a vector and an antibody according to any of the preceding claims, and in addition at least one excipient, carrier or diluent.

According to an embodiment, the invention concerns the vaccine composition, further comprising at least one adjuvant.

According to an embodiment, the invention concerns a medical composition comprising antibodies raised using a virus according to the invention.

According to an embodiment, the invention concerns a pharmaceutical composition comprising a mutated peptide, an envelope protein, a mutated envelope protein, an antigen, a nucleic acid sequence, a vector, an antibody or a vaccine composition according to any of the preceding claims, and at least one pharmaceutically acceptable excipient, diluents or carrier.

According to an embodiment, the invention concerns a use of a mutated peptide, an envelope protein, a mutated envelope protein, an antigen, a nucleic acid sequence, a vector or an antibody according to any of the preceding claims, for a medical purpose, such as for the treatment, amelioration or prevention of a clinical condition, such as for the manufacture of a medicament for the treatment, amelioration or prevention of a clinical condition.

According to an embodiment, the invention concerns a method of treating or ameliorating the symptoms of an individual, or prophylactic treating an individual, comprising administering an amount of mutated peptide, an envelope protein, a mutated envelope protein, antigen, nucleic acid sequence, vector or vaccine composition according to any of the preceding claims.

According to an embodiment, the invention may be used with human and/or animal vira.

Table 2 below, provides the location of a number of identified immunosuppressive domains.

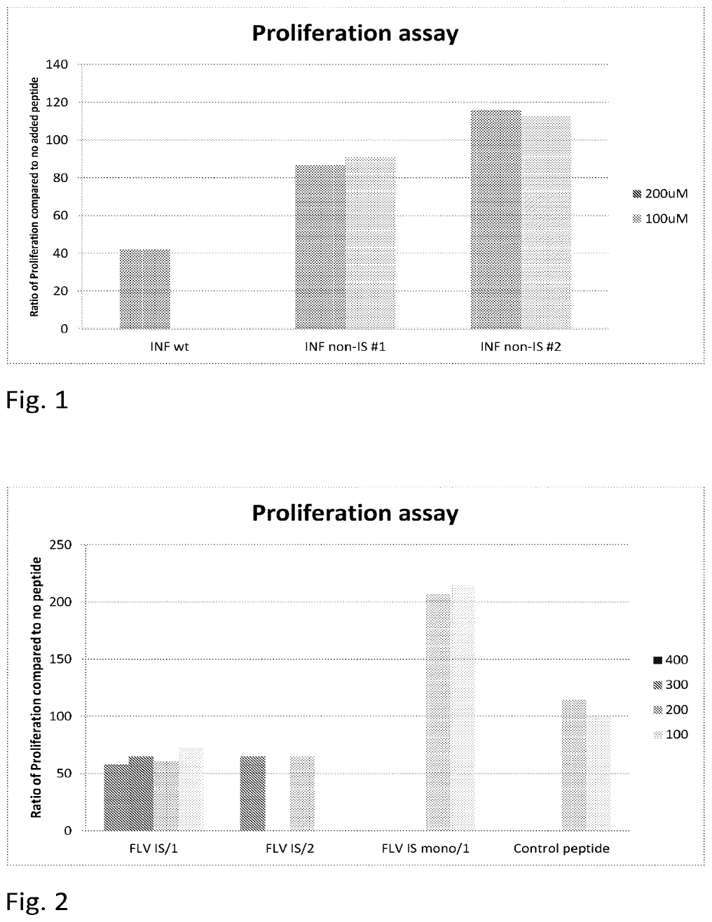

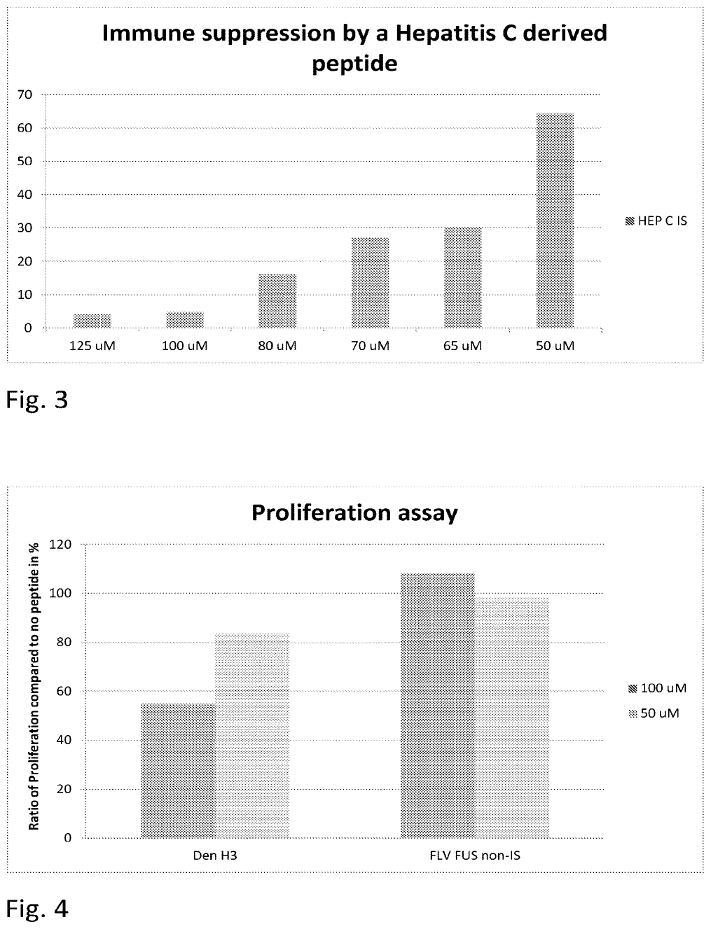

TABLE 2
Localization of identified immunosuppressive domains

| Family (-viridae), | | |
|---|---|---|
| Subfamily (-virinae), | | |
| Genus (-virus) or | | |
| Species (-virus) of | Localization of prototype | |
| viruses | immunosuppressive domain | Reference |
| All Flavirus | Protein E | Seligman S J. Constancy and |
| | 98-DRGWGNXCGXFGKGXX-113 | diversity in the flavivirus |
| | | fusion peptide. Virol J. 2008 |
| | | Feb 14; 5:27. |
| All Flavirus | Protein E | FIG. 1 |
| (e.g. Dengue 3) | 416-GDTAWDFGSVGGVLNSLGK-434 | Schmidt A G, Yang P L, Harrison S C. |
| | | Peptide inhibitors of |
| | | dengue-virus entry target a |
| | | late-stage fusion |
| | | intermediate. PLoS Pathog. |
| | | 2010 Apr 8; 6(4): e1000851. |
| Hepatitis C | E2 (SEQ ID NO: 3) | Albecka A, Montserret R, Krey T, |
| | 71-GLIHLHQNIVDVQYLYG-87 | Tarr A W, Diesis E, Ball J K, |
| | | Descamps V, |
| | | Duverlie G, Rey F, Penin F, |
| | | Dubuisson J. Identification of |
| | | new functional regions in |
| | | hepatitis C virus envelope |
| | | glycoprotein E2. J Virol. 2011 |
| | | Feb; 85(4): 1777-92. |
| | | Epub 2010 Dec 8. |
| Influenza A1-16 | HA2 (SEQ ID NO: 4) | Cross K J, Wharton S A, Skehel J J, |
| Influenza B | 1-GLFGAIAGFIENGWEG-16 | Wiley D C, Steinhauer D A. |
| | | Studies on influenza |
| | | haemagglutinin fusion peptide |
| | | mutants generated by reverse |
| | | genetics. EMBO J. 2001 |
| | | Aug 15; 20(16): 4432-42. |

According to an embodiment, an immunosuppressive domain may be identified by its position, e.g. as indicated in Table 2.

According to an embodiment, the invention concerns an immunosuppressive domain identified by its position.

According to an embodiment, the invention concerns an immunosuppressive domain identified by its secondary, tertiary or quaternary structure in the folded fusion protein.

According to an embodiment, the invention concerns an entity selected among the group consisting of a mutated peptide, an envelope protein, a mutated envelope protein, an antigen, a nucleic acid sequence and a vector, wherein an immunosuppressive domain identified by its position, has been modified or mutated in order to suppress its immunosuppressive properties.

All cited references are incorporated by reference.

The accompanying Figures and Examples are provided to explain rather than limit the present invention. It will be clear to the person skilled in the art that aspects, embodiments and claims of the present invention may be combined.

### EXAMPLES

### Peptide Solutions

The peptides were either dissolved in water or in cases of low water solubility, 5% DMSO solutions were used to dissolve the peptides.

### Assay to Measure the Immunosuppressive Activity of Peptides Derived From Viral Surface Proteins or Their Mutants

The peptides can be prepared by different means including, but not limited to, solid phase synthesis commonly used for such purposes. The peptides can be dimerized using a cysteine residue either at the N- or C-terminal or in the middle of the peptide or by using any other molecule or atom that is covalently bound to peptide molecules.

The peptides can be coupled to a carrier protein such as BSA by covalent bounds including, but not limited to, disulfide bridges between the peptide cysteine residues and the carrier protein or through amino groups including those in the side chain or Lysine residues.

The peptides can have non-viral derived amino acids added to their C-terminal for increasing their water solubility.

### Assay to Test the Immunosuppressive Activity of Peptides

### Experiment Design

Human Peripheral Blood Mononuclear Cells (PBMC) are prepared freshly from healthy donors. These are stimulated by Con A (5 ug/mL) concomitant to peptide addition at different concentrations (i.e. 25 uM, 50 uM and 100 uM). Cultures are maintained and lymphocyte proliferation is measured 72 hrs later by EdU incorporation and Click-iT labelling with Oregon Green (Invitrogen, Denmark) as recommended by the manufacturer. The degree of activated lymphocytes is proportional to the fluorescence detection.

### CTLL-2 Assay

100.000 CTLL-2 cells are seeded pr. well in a 48 well-plate (Nunc) in 200 uL of medium (RPMI+2 mM L-glutamine+1 mM Na-pyruvat+10% FCS+0.5 ng/mL IL-2) 2 hours later the peptides are added to the wells. 24 h later the cells are labeled using the Click-it reaction kit (Invitrogen cat. #C35002). The fluorescence of the cells is measured on a flow cytometer. The degree of proliferation in each sample is proportional to the detected fluorescence.

### Test of Immunosuppression From Monomer and Dimeric Peptides

100.000 CTLL-2 cells were seeded pr. well in a 48 well-plate (Nunc) in 200 uL of medium (RPMI+2 mM L-glutamine+1 mM Na-pyruvat+10% FCS+0.5 ng/mL IL-2) 2 hours later the peptides were added to the wells. 24 h later the cells were labeled using the Click-it reaction kit (Invitrogen cat. #C35002). The fluorescence of the cells was measured on a flow cytometer. The degree of proliferation in each sample is proportional to the detected fluorescence.

### Quantification of Proliferation Inhibition

The degree of inhibition of proliferation of CTLL-2 cells is visualized in the diagrams in the figures. The ratios are calculated by dividing the number of labeled cells (growing cells) in cultures in presence of peptide with cultures in absence of peptides, but added the same volume of the solute that was used to dissolve the peptides. That is in cases where the peptides were dissolved in 5% DMSO, the same volume of 5% DMSO was added to the control cells.

### Appendix

### Classes of Enveloped RNA Viruses That Contain Human Pathogens

### Flaviridae (Type II Fusion)

Flaviviridae have monopartite, linear, single-stranded RNA genomes of positive polarity, 9.6- to 12.3-kilobase in length. Virus particles are enveloped and spherical, about 40-60 nm in diameter.

Major diseases caused by the Flaviviridae family include:
Dengue fever
Japanese encephalitis
Kyasanur Forest disease
Murray Valley encephalitis
St. Louis encephalitis
Tick-borne encephalitis
West Nile encephalitis
Yellow fever
Hepatitis C Virus Infection

### Existing Vaccines for Flaviridae

The successful yellow fever 17D vaccine, introduced in 1937, produced dramatic reductions in epidemic activity. Effective killed Japanese encephalitis and Tick-borne encephalitis vaccines were introduced in the middle of the 20th century. Unacceptable adverse events have prompted change from a mouse-brain killed Japanese encephalitis vaccine to safer and more effective second generation Japanese encephalitis vaccines. These may come into wide use to effectively prevent this severe disease in the huge populations of Asia—North, South and Southeast. The dengue viruses produce many millions of infections annually due to transmission by a successful global mosquito vector. As mosquito control has failed, several dengue vaccines are in varying stages of development. A tetravalent chimeric vaccine that splices structural genes of the four dengue viruses onto a 17D yellow fever backbone is in Phase III clinical testing.

### Genus Flavivirus

Flaviviruses share a common size (40-65 nm), symmetry (enveloped, icosahedral nucleocapsid), nucleic acid (positive-sense, single stranded RNA approximately 10,000-11,000 bases), and appearance in the electron microscope.

These viruses are transmitted by the bite from an infected arthropod (mosquito or tick). Human infections with these viruses are typically incidental, as humans are unable to replicate the virus to high enough titres to reinfect arthropods and thus continue the virus life cycle. The exceptions to this are yellow fever and dengue viruses, which still require mosquito vectors, but are well-enough adapted to humans as to not necessarily depend upon animal hosts (although both continue have important animal transmission routes as well).

Genus Hepacivirus (type species Hepatitis C virus, the single member)

Hepatitis C is an infectious disease affecting the liver, caused by the hepatitis C virus (HCV). The infection is often asymptomatic, but once established, chronic infection can progress to scarring of the liver (fibrosis), and advanced scarring (cirrhosis), which is generally apparent after many years. In some cases, those with cirrhosis will go on to develop liver failure or other complications of cirrhosis, including liver cancer or life threatening esophageal varices and gastric varices. The hepatitis C virus is spread by blood-to-blood contact. Most people have few, if any symptoms after the initial infection, yet the virus persists in the liver in about 85% of those infected. Persistent infection can be treated with medication, peg-interferon and ribavirin being the standard-of-care therapy. Overall, 51% are cured. Those who develop cirrhosis or liver cancer may require a liver transplant, and the virus universally recurs after the transplant takes place. An estimated 180 million people worldwide are infected with hepatitis C. Hepatitis C is not known to cause disease in other animals. No vaccine against hepatitis C is currently available. The existence of hepatitis C (originally “non-A non-B hepatitis”) was postulated in the 1970s and proven in 1989. It is one of five known hepatitis viruses: A, B, C, D, and E.

The hepatitis C virus is a small (50 nm in size), enveloped, single-stranded, positive sense RNA virus. There are six major genotypes of the hepatitis C virus, which are indicated numerically (e.g., genotype 1, genotype 2, etc.). Based on the NS5 gene there are three major and eleven minor genotypes. The major genotypes diverged about 300-400 years ago from the ancestor virus. The minor genotypes diverged about 200 years ago from their major genotypes. All of the extant genotypes appear to have evolved from genotype 1 subtype 1b.

The hepatitis C virus is transmitted by blood-to-blood contact. In developed countries, it is estimated that 90% of persons with chronic HCV infection were infected through transfusion of unscreened blood or blood products or via injecting drug use or sexual exposure. In developing countries, the primary sources of HCV infection are unsterilized injection equipment and infusion of inadequately screened blood and blood products.

### Genus Pestivirus

### TogaviridaeType II Fusion

The Togaviridae are a family of viruses, including the following genera:

### Genus Alphavirus

Alphaviruses have a positive sense single stranded RNA genome. There are 27 alphaviruses, able to infect various vertebrates such as humans, rodents, fish, birds, and larger mammals such as horses as well as invertebrates. Transmission between species and individuals occurs mainly via mosquitoes making the alphaviruses a contributor to the collection of Arboviruses- or Arthropod Borne Viruses. Alphaviruses particles are enveloped, have a 70 nm diameter, tend to be spherical and have a 40 nm isometric nucleocapsid.

There are two open reading frames (ORF's) in the genome, non-structural and structural. The first is non structural and encodes proteins for transcription and replication of viral RNA, and the second encodes three structural proteins: the core nucleocapsid protein C, and the envelope proteins P62 and E1 that associate as a heterodimer. The viral membrane-anchored surface glycoproteins are responsible for receptor recognition and entry into target cells through membrane fusion. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the centre to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphaviral glycoprotein E1 is a class II viral fusion protein. The structure of the Semliki Forest virus revealed a structure that is similar to that of flaviviral glycoprotein E, with three structural domains in the same primary sequence arrangement. The E2 glycoprotein functions to interact with the nucleocapsid through its cytoplasmic domain, while its ectodomain is responsible for binding a cellular receptor. Most alphaviruses lose the peripheral protein E3, but in Semliki viruses it remains associated with the viral surface.

### Genus Rubivirus

### Genus Rubivirus

### Bunyaviridae Type II Fusion Mechanism

Bunyaviridae is a family of negative-stranded RNA viruses. Though generally found in arthropods or rodents, certain viruses in this family occasionally infect humans. Some of them also infect plants.

Bunyaviridae are vector-borne viruses. With the exception of Hantaviruses, transmission occurs via an arthropod vector (mosquitos, tick, or sandfly). Hantaviruses are transmitted through contact with deer mice feces. Incidence of infection is closely linked to vector activity, for example, mosquito-borne viruses are more common in the summer.

Human infections with certain Bunyaviridae, such as Crimean-Congo hemorrhagic fever virus, are associated with high levels of morbidity and mortality, consequently handling of these viruses must occur with a Biosafety level 4 laboratory. They are also the cause of severe fever with thrombocytopenia syndrome.

Hanta virus or Hantavirus Hemorrhagic fever, common in Korea, Scandinavia, Russia, and the American southwest, is associated with high fever, lung edema and pulmonary failure. Mortality is around 55%.

The antibody reaction plays an important role in decreasing levels of viremia.

Genus Hantavirus; type species: Hantaan virus

Hantaviruses are negative sense RNA viruses in the Bunyaviridae family. Humans may be infected with hantaviruses through rodent bites, urine, saliva or contact with rodent waste products. Some hantaviruses cause potentially fatal diseases in humans, hemorrhagic fever with renal syndrome (HFRS) and hantavirus pulmonary syndrome (HPS), but others have not been associated with human disease. HPS cannot be transmitted person-to-person. The name hantavirus is derived from the Hantan River area in South Korea, which provided the founding member of the group: Hantaan virus (HTNV), isolated in the late 1970s by Ho-Wang Lee and colleagues. HTNV is one of several hantaviruses that cause HFRS, formerly known as Korean hemorrhagic fever.

### Genus Ortho-Bunya-Virus

The orthobunyaviruses are maintained in nature by sylvatic transmission cycles between hematophagous mosquitoes and susceptible mammalian hosts, principally rodents and other small mammals. Several members of the California serogroup of orthobunyaviruses, including La Crosse (LAC) and Tahyna (TAH) viruses, are significant human pathogens. LAC virus is an important cause of pediatric encephalitis and aseptic meningitis in the Midwestern United States where approximately 100 cases are reported annually; TAH virus, indigenous to central Europe, is associated with influenzalike febrile illnesses. La Crosse virus is a NIAID Category B priority pathogen.

The orthobunyaviruses are enveloped, negative-stranded RNA viruses with a tripartite genome comprised of large (L), medium (M), and small (S) segments The M segment encodes three proteins in a single open reading frame (ORF): two surface transmembrane glycoproteins, herein referred to as Gn (G2) and Gc (G1), respectively, to delineate their order in the precursor polyprotein, and NSm, a protein of unknown function. Gn and Gc are thought to associate as a heteromultimer after cleavage of the polyprotein.

Genus Phlebovirus; type species: Rift Valley fever virus

Phlebovirus is one of five genera of the family Bunyaviridae. The Phlebovirus genus currently comprises over 70 antigenically distinct serotypes, only a few of which have been studied. The 68 known serotypes are divided into two groups: the Phlebotomus fever viruses (the sandfly group, transmitted by Phlebotominae sandflies) comprises 55 members and the Uukuniemi group (transmitted by ticks) comprises the remaining 13 members.

Of these 68 serotypes, eight of them have been linked to disease in humans. They are: Alenquer virus, Candiru virus, Chagres virus, Naples virus, Punta Toro virus, Rift Valley fever, Sicilian virus, and Toscana virus. Recently identified is another human pathogenic serotype, the SFTS virus.

Rift Valley Fever (RVF) is a viral zoonosis (affects primarily domestic livestock, but can be passed to humans) causing fever. It is spread by the bite of infected mosquitoes, typically the Aedes or Culex genera. The disease is caused by the RVF virus, a member of the genus Phlebovirus (family Bunyaviridae). The disease was first reported among livestock in Kenya around 1915, but the virus was not isolated until 1931. RVF outbreaks occur across sub-Saharan Africa, with outbreaks occurring elsewhere infrequently (but sometimes severely—in Egypt in 1977-78, several million people were infected and thousands died during a violent epidemic. In Kenya in 1998, the virus claimed the lives of over 400 Kenyans. In September 2000 an outbreak was confirmed in Saudi Arabia and Yemen).

In humans the virus can cause several different syndromes. Usually sufferers have either no symptoms or only a mild illness with fever, headache, myalgia and liver abnormalities. In a small percentage of cases (<2%) the illness can progress to hemorrhagic fever syndrome, meningoencephalitis (inflammation of the brain), or affecting the eye. Patients who become ill usually experience fever, generalized weakness, back pain, dizziness, and weight loss at the onset of the illness. Typically, patients recover within 2-7 days after onset.

Approximately 1% of human sufferers die of the disease. Amongst livestock the fatality level is significantly higher. In pregnant livestock infected with RVF there is the abortion of virtually 100% of fetuses. An epizootic (animal disease epidemic) of RVF is usually first indicated by a wave of unexplained abortions.

### Orthomyxoviridae Type I Fusion

The Orthomyxoviridae (orthos, Greek for “straight”; myxa, Greek for “mucus”)^{]} are a family of RNA viruses that includes five genera: Influenzavirus A, Influenzavirus B, Influenzavirus C, Isavirus and Thogotovirus. A sixth has recently been described. The first three genera contain viruses that cause influenza in vertebrates, including birds (see also avian influenza), humans, and other mammals. Isaviruses infect salmon; thogotoviruses infect vertebrates and invertebrates, such as mosquitoes and sea lice.

The three genera of Influenzavirus, which are identified by antigenic differences in their nucleoprotein and matrix protein infect vertebrates as follows:
Influenzavirus A infects humans, other mammals, and birds, and causes all flu pandemics
Influenzavirus B infects humans and seals
Influenzavirus C infects humans and pigs

### Paramyxoviridae Type I Fusion Mechanism

The fusion protein F projects from the envelope surface as a trimer, and mediates cell entry by inducing fusion between the viral envelope and the cell membrane by class I fusion. One of the defining characteristics of members of the paramyxoviridae family is the requirement for a neutral pH for fusogenic activity. A number of important human diseases are caused by paramyxoviruses. These include mumps, measles, which caused 745,000 deaths in 2001 and respiratory syncytial virus (RSV) which is the major cause of bronchiolitis and pneumonia in infants and children. The parainfluenza viruses are the second most common causes of respiratory tract disease in infants and children. They can cause pneumonia, bronchitis and croup in children and the elderly.

Human metapneumovirus, initially described in about 2001, is also implicated in bronchitis, especially in children.

genus Paramyxoviruses are also responsible for a range of diseases in other animal species, for example canine distemper virus (dogs), phocine distemper virus (seals), cetacean morbillivirus (dolphins and porpoises) Newcastle disease virus (birds), and rinderpest virus (cattle). Some paramyxoviruses such as the henipaviruses are zoonotic pathogens, occurring naturally in an animal host, but also able to infect humans.

Hendra virus (HeV) and Nipah virus (NiV) in the genus Henipavirus have emerged in humans and livestock in Australia and Southeast Asia. Both viruses are contagious, highly virulent, and capable of infecting a number of mammalian species and causing potentially fatal disease. Due to the lack of a licensed vaccine or antiviral therapies, HeV and NiV are designated as biosafety level (BSL) 4 agents. The genomic structure of both viruses is that of a typical paramyxovirus.

### Genus Pneumovirinae

Genus Pneumovirus (type species Human respiratory syncytial virus, others include Bovine respiratory syncytial virus)
Human respiratory syncytial virus (RSV) is a virus that causes respiratory tract infections. It is the major cause of lower respiratory tract infections and hospital visits during infancy and childhood. A prophylactic medication (not a vaccine) exists for preterm birth (under 35 weeks gestation) infants and infants with a congenital heart defect (CHD) or bronchopulmonary dysplasia (BPD). Treatment is limited to supportive care, including oxygen therapy.
In temperate climates there is an annual epidemic during the winter months. In tropical climates, infection is most common during the rainy season.
In the United States, 60% of infants are infected during their first RSV season and nearly all children will have been infected with the virus by 2-3 years of age. en.wikipedia.org/wiki/Respiratory_syncytial_virus—cite_note-Glezen86-0 Of those infected with RSV, 2-3% will develop bronchiolitis, necessitating hospitalization Natural infection with RSV induces protective immunity which wanes over time—possibly more so than other respiratory viral infections—and thus people can be infected multiple times. Sometimes an infant can become symptomatically infected more than once, even within a single RSV season. Severe RSV infections have increasingly been found among elderly patients.
RSV is a negative-sense, single-stranded RNA virus of the family Paramyxoviridae, which includes common respiratory viruses such as those causing measles and mumps. RSV is a member of the paramyxovirus subfamily Pneumovirinae. Its name comes from the fact that F proteins on the surface of the virus cause the cell membranes on nearby cells to merge, forming syncytia.

### Coronaviriridae Type I Fusion

Coronaviruses primarily infect the upper respiratory and gastrointestinal tract of mammals and birds. Four to five different currently known strains of coronaviruses infect humans. The most publicized human coronavirus, SARS-CoV which causes SARS, has a unique pathogenesis because it causes both upper and lower respiratory tract infections and can also cause gastroenteritis. Coronaviruses are believed to cause a significant percentage of all common colds in human adults. Coronaviruses cause colds in humans primarily in the winter and early spring seasons. The significance and economic impact of coronaviruses as causative agents of the common cold are hard to assess because, unlike rhinoviruses (another common cold virus), human coronaviruses are difficult to grow in the laboratory.

Coronaviruses also cause a range of diseases in farm animals and domesticated pets, some of which can be serious and are a threat to the farming industry. Economically significant coronaviruses of farm animals include porcine coronavirus (transmissible gastroenteritis coronavirus, TGE) and bovine coronavirus, which both result in diarrhea in young animals. Feline Coronavirus: 2 forms, Feline enteric coronavirus is a pathogen of minor clinical significance, but spontaneous mutation of this virus can result in feline infectious peritonitis (FIP), a disease associated with high mortality. There are two types of canine coronavirus (CCoV), one that causes mild gastrointestinal disease and one that has been found to cause respiratory disease. Mouse hepatitis virus (MHV) is a coronavirus that causes an epidemic murine illness with high mortality, especially among colonies of laboratory mice. Prior to the discovery of SARS-CoV, MHV had been the best-studied coronavirus both in vivo and in vitro as well as at the molecular level. Some strains of MHV cause a progressive demyelinating encephalitis in mice which has been used as a murine model for multiple sclerosis. Significant research efforts have been focused on elucidating the viral pathogenesis of these animal coronaviruses, especially by virologists interested in veterinary and zoonotic diseases.

### SARS-Coronavirus

SARS is most closely related to group 2 coronaviruses, but it does not segregate into any of the other three groups of coronaviruses. SARS was determined to be an early split off from the group 2 coronaviruses based on a set of conserved domains that it shares with group 2. A main difference between group 2 coronovirus and SARS is the nsp3 replicase subunit encoded by ORF1a. SARS does not have a papain-like proteinase 1.

### Arenaviridae: Glycoprotein G2 is a Type I Fusion

Arenavirus is a genus of virus that infects rodents and occasionally humans. At least eight Arenaviruses are known to cause human disease. The diseases derived from Arenaviruses range in severity. Aseptic meningitis, a severe human disease that causes inflammation covering the brain and spinal cord, can arise from the Lymphocytic choriomeningitis virus (LCMV) infection. Hemorrhagic fever syndromes are derived from infections such Guanarito virus (GTOV), Junin virus (JUNV), Lassa virus (LASV) causing Lassa fever, Machupo virus (MACV), Sabia virus (SABV), or Whitewater Arroyo virus (WWAV).^{[1]} Arenaviruses are divided into two groups; the Old World or New World. The differences between these groups are distinguished geographically and genetically. Because of the epidemiological association with rodents, some arenaviruses and bunyaviruses are designated as Roboviruses.
LCMV-Lassa virus (Old World) complex:
   Ippy virus
   Lassa virus
   Lujo virus
   Lymphocytic choriomeningitis virus

LCMV infection manifests itself in a wide range of clinical symptoms, and may even be asymptomatic for immunocompetent individuals. Onset typically occurs between one or two weeks after exposure to the virus and is followed by a biphasic febrile illness. During the initial or prodromal phase, which may last up to a week, common symptoms include fever, lack of appetite, headache, muscle aches, malaise, nausea, and/or vomiting. Less frequent symptoms include a sore throat and cough, as well as joint, chest, and parotid pain. The onset of the second phase occurs several days after recovery, and consists of symptoms of meningitis or encephalitis. Pathological findings during the first stage consist of leukopenia and thrombocytopenia. During the second phase, typical findings include elevated protein levels, increased leukocyte count, or a decrease in glucose levels of the cerebrospinal fluid).

### Congenital Infection

Lymphocytic choriomeningitis is a particular concern in obstetrics, as vertical transmission is known to occur. For immunocompetent mothers, there is no significant threat, but the virus has damaging effects upon the fetus. If infection occurs during the first trimester, LCMV results in an increased risk of spontaneous abortion. Later congenital infection may lead to malformations such as chorioretinitis, intracranial calcifications, hydrocephalus, microcephaly or macrocephaly, mental retardation, and seizures. Other findings include chorioretinal scars, optic atrophy, and cataracts. Mortality among infants is approximately 30%. Among the survivors, two thirds have lasting neurologic abnormalities. If a woman has come into contact with a rodent during pregnancy and LCM symptoms are manifested, a blood test is available to determine previous or current infection. A history of infection does not pose a risk for future pregnancies.

### Human-to-Human Transmission Through Organ Donation

In May 2005, four solid-organ transplant recipients contracted an illness that was later diagnosed as lymphocytic choriomeningitis. All received organs from a common donor, and within a month of transplantation, three of the four recipients had died as a result of the viral infection. Epidemiologic investigation traced the source to a pet hamster that the organ donor had recently purchased from a Rhode Island pet store. A similar case occurred in Massachusetts in 2008. Currently, there is not a LCMV infection test that is approved by the Food and Drug Administration for organ donor screening. The Morbidity and Mortality Weekly Report advises health-care providers to “consider LCMV infection in patients with aseptic meningitis and encephalitis and in organ transplant recipients with unexplained fever, hepatitis, or multisystem organ failure.”

### Hepadnaviridae: Fusion Mechanism Neither Type I nor Type II

Hepadnaviruses are a family of viruses which can cause liver infections in humans and animals. There are two recognized genera

Hepadnaviruses have very small genomes of partially double-stranded, partially single stranded circular DNA. The genome consists of two uneven strands of DNA. One has a negative-sense orientation, and the other, shorter, strand has a positive-sense orientation.

As it is a group 7 virus, replication involves an RNA intermediate. Three main open reading frames are encoded (ORFs) and the virus has four known genes which encode the core protein, the virus polymerase, surface antigens (preS1, preS2, and S) and the X protein. The X protein is thought to be non-structural; however, its function and significance are poorly understood.

### Rhabdoviridae Fusion Mechanism Neither Type I nor Type II

Rhabdoviruses carry their genetic material in the form of negative-sense single-stranded RNA. They typically carry genes for five proteins: large protein (L), glycoprotein (G), nucleoprotein (N), phosphoprotein (P), and matrix protein (M). Rhabdoviruses that infect vertebrates are bullet-shaped. The prototypical and best studied rhabdovirus is vesicular stomatitis virus.

Rhabdoviruses are important pathogens of animals and plants. Rhabdoviruses include RaV (Rabies virus), VSV (Vesicular stomatitis virus). Rhabdoviruses are transmitted to hosts by arthropods, such as aphids, planthoppers, leafhoppers, black flies, sandflies, and mosquitoes.

The content of the XML file of the sequence listing named “Sequence-Listing-ST26-12397-0504.xml”, having a size of 355 kb and a creation date of 8 Jan. 2024, and electronically submitted via Patent Center on 8 Jan. 2024, is incorporated herein by reference in its entirety.

### ADDITIONAL REFERENCES

1. Sapir, A., et al., Viral and developmental cell fusion mechanisms: conservation and divergence. Dev Cell, 2008. 14(1): p. 11-21.
2. Cianciolo, G.J., et al., Murine malignant cells synthesize a 19,000-dalton protein that is physicochemically and antigenically related to the immunosuppressive retroviral protein, P15E. J Exp Med, 1983. 158(3): p. 885-900.
3. Hebebrand, L.C., et al., Inhibition of human lymphocyte mitogen and antigen response by a 15,000-dalton protein from feline leukemia virus. Cancer Res, 1979. 39(2 Pt 1): p. 443-7.
4. Cianciolo, G.J., et al., Macrophage accumulation in mice is inhibited by low molecular weight products from murine leukemia viruses. J Immunol, 1980. 124(6): p. 2900-5.
5. Mangeney, M. and T. Heidmann, Tumor cells expressing a retroviral envelope escape immune rejection in vivo. Proc Natl Acad Sci USA, 1998. 95(25): p. 14920-5.
6. Mangeney, M., et al., Placental syncytins: Genetic disjunction between the fusogenic and immunosuppressive activity of retroviral envelope proteins. Proc Natl Acad Sci USA, 2007. 104(51): p. 20534-9.
7. Cianciolo, G.J., et al., Inhibition of lymphocyte proliferation by a synthetic peptide homologous to retroviral envelope proteins. Science, 1985. 230(4724): p. 453-5.
8. Cianciolo, G.J., H. Bogerd, and R. Snyderman, Human retrovirus-related synthetic peptides inhibit T lymphocyte proliferation. Immunol Lett, 1988. 19(1): p. 7-13.
9. Yaddanapudi, K., et al., Implication of a retrovirus-like glycoprotein peptide in the immunopathogenesis of Ebola and Marburg viruses. Faseb J, 2006. 20(14): p. 2519-30.
10. Haraguchi, S., et al. Differential modulation of Th1-and Th2-related cytokine mRNA expression by a synthetic peptide homologous to a conserved domain within retroviral envelope protein. Proc Natl Acad Sci USA, 1995. 92, 3611-15.
11. Harrell, R.A., et al Cianciolo. Suppression of the respiratoryburst of human monocytes by a synthetic peptide homologous to envelope proteins of human and animal retroviruses. J Immunol, 1986. 136, 3517-520.
12. Kleinerman, E.S., et al. Lachman. A synthetic peptide homologous to the envelope proteins of retroviruses inhibits monocyte-mediated killing by inactivating interleukin 1. J Immunol, 1987. 139, 2329-337.
13. Schlecht-Louf G,. et al. Retro viral infection in vivo requires an immune escape virulence factor encrypted in the envelope protein of oncoretroviruses. Proc Natl Acad Sci USA. 2010 Feb. 23; 107(8):3782-7.
14. Volchkov VE et al. The envelope glycoprotein of Ebola virus contains an 25 immunosuppressive-like domain similar to oncogenic retroviruses. FEBS Lett. 1992 Jul. 6; 305(3):181-4.
15. Cross KJ, Wharton SA, Skehel JJ, Wiley DC, Steinhauer DA. Studies on influenza haemagglutinin fusion peptide mutants generated by reverse genetics. EMBO J. 2001 Aug. 15; 20(16):4432-42.

1. (canceled)

2. A synthetic peptide comprising a dimeric form of an amino acid sequence, the amino acid sequence comprising a variant of SEQ ID NO:4, said variant differing from SEQ ID NO:4 at 1 or 2 or 3 point mutation(s), wherein each of said point mutation(s) consists of at least one exchange of an amino acid of SEQ ID NO:4 with another amino acid.

3. The synthetic peptide according to claim 2, comprising a cysteine residue at an N-terminal or C-terminal position of the amino acid sequence or synthetic peptide.

4. The synthetic peptide according to claim 3, wherein said amino acid sequence is dimerized through said N-terminal or C-terminal cysteine residue.

5. A synthetic peptide comprising a dimeric form of an amino acid sequence, the amino acid sequence comprising a variant of SEQ ID NO:214, said variant differing from SEQ ID NO:214 at 1 or 2 or 3 or 4 or 5 or 6 or 7 or 8 or 9 or 10 or 11 point mutation(s).

6. The synthetic peptide according to claim 5, wherein said amino acid sequence comprises 1 or 2 or 3 point mutation(s).

7. The synthetic peptide according to claim 5, comprising a cysteine residue at an N-terminal or C-terminal position of the amino acid sequence or synthetic peptide.

8. The synthetic peptide according to claim 7, wherein said amino acid sequence is dimerized through said N-terminal or C-terminal cysteine residue.

9. The synthetic peptide according to claim 5, wherein said amino acid sequence has at least 75% sequence identity to SEQ ID NO:214.

10. The synthetic peptide according to claim 9, comprising a cysteine residue at an N-terminal or C-terminal position of the amino acid sequence or synthetic peptide

11. The synthetic peptide according to claim 10, wherein said amino acid sequence is dimerized through said N-terminal or C-terminal cysteine residue.

12. A nucleic acid sequence encoding the synthetic peptide according to claim 2.

13. A pharmaceutical composition comprising the synthetic peptide according to claim 2.

14. The pharmaceutical composition according to claim 13, further comprising at least one pharmaceutically acceptable excipient, diluent or carrier.
